SEQUENCE LISTING (54 sequences)

SEQ ID NO: 1 - DNA, 750 bp, Artificial Sequence
  anti-mCD8 YTS 169 cDb nucleic acid sequence
gaagtgaagctgcaggaaagcggcggaggcctggtgcagcccggcagaagcctgaagctgagctgtgccgccagcggcttcaacttcaacgactactggatgggctgggtccgacaggctcctggcaagggcctggaatggatcggcgagatcaacaaggacagcagcaccatcaactacacccccagcctgaaggacaagttcaccatcagcagagacaacgcccagaacaccctgtacctgcagatgagcaagctgggcagcgaggacaccgccatctactactgcgccagagccagaggcatgatggtgctgatcatcccccactacttcgactactggggccagggcgtgatggtcaccgtgtccagcggcggcggaggaagcgacatcgtgctgacccagagccccgctatggccatgagccctggcgagagaatcacaatcagctgcagagccagcgagagcgtgtccaccagaatgcactggtatcagcagaagcccggccagcagcccaagctgctgatctacggcgccagcaacctggaatccggcgtgccagccagattcagcggcagcggctccggcaccgacttcaccctgaccatcgaccccgtggaagccaacgacaccgccacctatttctgccagcagtcttggtacgacccctggaccttcggtggaggcaccaagctggaactgaaggcggccgcaggctgcggaggccaccaccatcatcaccattgatag

SEQ ID NO: 2 - protein, 248 aa, Artificial Sequence
  anti-mCD8 YTS 169 cDb amino acid sequence
EVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSGGGGSDIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELKAAAGCGGHHHHHH

SEQ ID NO: 3 - DNA, 732 bp, Artificial Sequence
  anti-mCD8 2.43 cDb nucleic acid sequence
gaagtgcagctggtggaaagcggcggaggcctggtgcagcccggcagaagcctgaagctgagctgtgccgccagcggcttcaccttcagcaactactacatggcttgggtgcgccaggcccccaccaagggactggaatgggtggcctacatcaacaccggcggaggcaccacctactacagagacagcgtgaagggcagattcaccatcagcagggacgacgccaagagcaccctgtacctgcagatggacagcctgagaagcgaggacaccgctacctactactgcaccaccgccatcggctactacttcgactactggggccagggcgtgatggtgacagtgtccagcggtggcggaggaagcgacatccagctgacacagagccccgccagcctgagcgcctctctgggcgagacagtgtctatcgagtgcctggccagcgaggacatctacagctacctggcctggtatcagcagaagcccggcaagagcccccaggtgctgatctacgccgccaacagactgcaggacggcgtgcccagcagattcagcggctctggcagcggcacccagtacagcctgaagatcagcggcatgcagcccgaggacgagggcgactacttctgtctgcagggcagcaagttcccctacaccttcggcgctggcaccaagctggaactgaaggcggccgcaggctgcggaggccaccaccatcatcaccattgatag

SEQ ID NO: 4 - protein, 242 aa, Artificial Sequence
  anti-mCD8 2.43 cDb amino acid sequence
EVQLVESGGGLVQPGRSLKLSCAASGFTFSNYYMAWVRQAPTKGLEWVAYINTGGGTTYYRDSVKGRFTISRDDAKSTLYLQMDSLRSEDTATYYCTTAIGYYFDYWGQGVMVTVSSGGGGSDIQLTQSPASLSASLGETVSIECLASEDIYSYLAWYQQKPGKSPQVLIYAANRLQDGVPSRFSGSGSGTQYSLKISGMQPEDEGDYFCLQGSKFPYTFGAGTKLELKAAAGCGGHHHHHH

SEQ ID NO: 5 - DNA, 729 bp, Artificial Sequence
  anti-mCD8 YTS 156 cDb nucleic acid sequence
gaagtgaagctgcaggaaagcggccccagcctggtgcagcctagccagaccctgagcctgacctgcagcgtgtccggcttcagcctgatcagcgacagcgtgcactgggtccgacagcctcccggcaagggcctggaatggatgggcggcatctgggccgacggctccaccgactacaacagcgccctgaagtccagactgagcatcagcagagacaccagcaagagccagggcttcctgaagatgaacagcctgcagaccgacgacaccgccatctatttctgcaccagcaaccgcgagagctactacttcgactactggggccagggcgtgatggtcaccgtgtccagcggcggcggaggctctgacatccagatgacccagagccctgccagcctgagcgccagcctgggcgacaaagtgaccatcacctgtcaggccagccagaacatcgacaagtatatcgcctggtatcagcagaagcctggcaaggcccccagacagctgatccactacaccagcacactggtgtccggcacccccagcagattcagcggcagcggttccggcagagactacagcttcagcatcagctccgtggaaagcgaggatatcgccagctactactgcctgcagtacgacaccctgtacaccttcggcgctggcaccaagctggaactgaaggcggccgcaggctgcggaggccaccaccatcatcaccattgatag

SEQ ID NO: 6 - protein, 240 aa, Artificial Sequence
  anti-mCD8 YTS 156 cDb amino acid sequence
EVKLQESGPSLVQPSQTLSLTCSVSGFSLISDSVHWVRQPPGKGLEWMGGIWADGSTDYNSALKSRLSISRDTSKSQGFLKMNSLQTDDTAIYFCTSNRESYYFDYWGQGVMVTVSSGGGGSDIQMTQSPASLSASLGDKVTITCQASQNIDKYIAWYQQKPGKAPRQLIHYTSTLVSGTPSRFSGSGSGRDYSFSISSVESEDIASYYCLQYDTLYTFGAGTKLELKAAAGCGHHHHHH

SEQ ID NO: 7 - protein, 124 aa, Artificial Sequence
  VH of the anti-mCD8 YTS 169 antibody amino acid
EVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSS

SEQ ID NO: 8 - protein, 7 aa, Artificial Sequence
  VH of the anti-mCD8 YTS 169 antibody amino acid sequence
GFNFNDY

SEQ ID NO: 9 - protein, 17 aa, Artificial Sequence
  CDR2 of the VH of the anti-mCD8 YTS 169 antibody amino acid sequence
EINKDSSTINYTPSLKD

SEQ ID NO: 10 - protein, 15 aa, Artificial Sequence
  CDR2 of the VH of the anti-mCD8 YTS 169 antibody amino acid sequence
ARGMMVLIIPHYFDY

SEQ ID NO: 11 - protein, 106 aa, Artificial Sequence
  VL of the anti-mCD8 YTS 169 antibody amino acid sequence
DIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELK

SEQ ID NO: 12 - protein, 11 aa, Artificial Sequence
  CDR1 of the VL of the anti-mCD8 YTS 169 antibody amino acid sequence
RASESVSTRMH

SEQ ID NO: 13 - protein, 7 aa, Artificial Sequence
  CDR2 of the VL of the anti-mCD8 YTS 169 antibody amino acid sequence
GASNLES

SEQ ID NO: 14 - protein, 9 aa, Artificial Sequence
  CDR3 of the VL of the anti-mCD8 YTS 169 antibody amino acid sequence
QQSWYDPWT

SEQ ID NO: 15 - protein, 117 aa, Artificial Sequence
  the VH of the anti-mCD8 2.43 antibody amino acid sequence
EVQLVESGGGLVQPGRSLKLSCAASGFTFSNYYMAWVRQAPTKGLEWVAYINTGGGTTYYRDSVKGRFTISRDDAKSTLYLQMDSLRSEDTATYYCTTAIGYYFDYWGQGVMVTVSS

SEQ ID NO: 16 - protein, 10 aa, Artificial Sequence
  CDR1 of the VH of the anti-mCD8 2.43 antibody amino acid sequence
GFTFSNYYMA

SEQ ID NO: 17 - protein, 15 aa, Artificial Sequence
  CDR2 of the VH of the anti-mCD8 2.43 antibody amino acid sequence
YINTGGGTTYYRDSV

SEQ ID NO: 18 - protein, 8 aa, Artificial Sequence
  CDR3 of the VH of the anti-mCD8 2.43 antibody amino acid sequence
AIGYYFDY

SEQ ID NO: 19 - protein, 107 aa, Artificial Sequence
  VL of the anti-mCD8 2.43 antibody amino acid sequence
DIQLTQSPASLSASLGETVSIECLASEDIYSYLAWYQQKPGKSPQVLIYAANRLQDGVPSRFSGSGSGTQYSLKISGMQPEDEGDYFCLQGSKFPYTFGAGTKLELK

SEQ ID NO: 20 - protein, 11 aa, Artificial Sequence
  CDR1 of the VL of the anti-mCD8 2.43 antibody amino acid sequence
LASEDIYSYLA

SEQ ID NO: 21 - protein, 7 aa, Artificial Sequence
  CDR2 of the VL of the anti-mCD8 2.43 antibody amino acid sequence
AANRLQD

SEQ ID NO: 22 - protein, 9 aa, Artificial Sequence
  CDR3 of the VL of the anti-mCD8 2.43 antibody amino acid sequence
LQGSKFPYT

SEQ ID NO: 23 - protein, 117 aa, Artificial Sequence
  VH of the anti-mCD8 YTS 156 antibody amino acid sequence
EVKLQESGPSLVQPSQTLSLTCSVSGFSLISDSVHWVRQPPGKGLEWMGGIWADGSTDYNSALKSRLSISRDTSKSQGFLKMNSLQTDDTAIYFCTSNRESYYFDYWGQGVMVTVSS

SEQ ID NO: 24 - protein, 10 aa, Artificial Sequence
  CDR1 of the VH of the anti-mCD8 YTS 156 antibody amino acid sequence
GFSLISDSVH

SEQ ID NO: 25 - protein, 16 aa, Artificial Sequence
  CDR1 of the VH of the anti-mCD8 YTS 156 antibody amino acid sequence
GSTDYNSALKSRLSIS

SEQ ID NO: 26 - protein, 9 aa, Artificial Sequence
  CDR1 of the VH of the anti-mCD8 YTS 156 antibody amino acid sequence
NRESYYFDY

SEQ ID NO: 27 - protein, 106 aa, Artificial Sequence
  VL of the anti-mCD8 YTS 156 antibody amino acid sequence
DIQMTQSPASLSASLGDKVTITCQASQNIDKYIAWYQQKPGKAPRQLIHYTSTLVSGTPSRFSGSGSGRDYSFSISSVESEDIASYYCLQYDTLYTFGAGTKLELK

SEQ ID NO: 28 - protein, 11 aa, Artificial Sequence
  VL of the anti-mCD8 YTS 156 antibody amino acid sequence
QASQNIDKYIA

SEQ ID NO: 29 - protein, 6 aa, Artificial Sequence
  CDR1 of the VL of the anti-mCD8 YTS 156 antibody amino acid sequence
TSTLVS

SEQ ID NO: 30 - protein, 8 aa, Artificial Sequence
  CDR1 of the VL of the anti-mCD8 YTS 156 antibody amino acid sequence
LQYDTLYT

SEQ ID NO: 31 - DNA, 26 bp, Artificial Sequence
  heavy chain CH1 primer
cggaattcaggggccatgggatagac

SEQ ID NO: 32 - DNA, 26 bp, Artificial Sequence
  kappa chain constant domain primer
cggaattcggatggtgggaagatgga

SEQ ID NO: 33 - protein, 29 aa, Artificial Sequence
  amino acid alpha helical linker
AGSADDARKDAGSKDDARKDDARKDGSSA

SEQ ID NO: 34 - DNA, 37 bp, Artificial Sequence
  sCD8a-F  primer
cacacagagctcaccggtaagccacaggcacccgaac

SEQ ID NO: 35 - DNA, 63 bp, Artificial Sequence
  sCD8(alpha)(beta)R primer
tgtgtgggatcccgcatcctttcttgcatcgtcggcagatcctgcagagttcactttctgaag

SEQ ID NO: 36 - DNA, 78 bp, Artificial Sequence
  sCD8b-F primer
cacacaggatccaaagatgacgcaaggaaggacgatgctaggaaggatggatcttccgcactcattcagaccccttcg

SEQ ID NO: 37 - DNA, 40 bp, Artificial Sequence
  sCD8b-R
tgtgtgtctagacgcggccgcaaccacagtcagcttcgtc

SEQ ID NO: 38 - DNA, 1290 bp, Artificial Sequence
  anti-mCD8 YTS 169 minibody nucleic acid sequence
tctagagccgccaccatggaaaccgacaccctgctgctgtgggtgctgctgctctgggtccccggaagcaccggtgaagtgaagctgcaggaaagcggcggaggcctggtgcagcccggcagaagcctgaagctgagctgtgccgccagcggcttcaacttcaacgactactggatgggctgggtccgacaggctcctggcaagggcctggaatggatcggcgagatcaacaaggacagcagcaccatcaactacacccccagcctgaaggacaagttcaccatcagcagagacaacgcccagaacaccctgtacctgcagatgagcaagctgggcagcgaggacaccgccatctactactgcgccagagccagaggcatgatggtgctgatcatcccccactacttcgactactggggccagggcgtgatggtcaccgtgtccagcggctctaccagtggcggaggatctggcggaggaagcggaggcggcggaagcagcgacatcgtgctgacccagagccccgctatggccatgagccctggcgagagaatcacaatcagctgcagagccagcgagagcgtgtccaccagaatgcactggtatcagcagaagcccggccagcagcccaagctgctgatctacggcgccagcaacctggaatccggcgtgccagccagattcagcggcagcggctccggcaccgacttcaccctgaccatcgaccccgtggaagccaacgacaccgccacctatttctgccagcagtcttggtacgacccctggaccttcggtggaggcaccaagctggaactgaagctcgagcccagaggccccaccatcaagccctgccctccctgcaagtgccctgcccctaacctgctgggcggacctggatctgtgcgggctccccaggtgtacgtgctgcccccacccgaggaagagatgaccaagaaacaggtgacactgacctgcatggtgacagacttcatgcccgaggatatctacgtggaatggaccaacaacggcaagaccgagctgaactacaagaacaccgagcccgtgctggacagcgacggcagctacttcatgtacagcaagctgcgggtggaaaagaaaaactgggtggaacggaacagctacagctgcagcgtggtgcacgagggcctgcacaactaccacaccaccaagagcttcagcaggacccccggcaaagcggccgcggccggccatctgcctgaaacaggggccggccaccaccaccatcaccatgcggccgcttgagaattc

SEQ ID NO: 39 - protein, 427 aa, Artificial Sequence
  anti-mCD8 YTS 169 minibody amino acid sequence
SRAATMETDTLLLWVLLLWVPGSTGEVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSGSTSGGGSGGGSGGGGSSDIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELKLEPRGPTIKPCPPCKCPAPNLLGGPGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNYHTTKSFSRTPGKAAAAGHLPETGAGHHHHHHAAA

SEQ ID NO: 40 - DNA, 828 bp, Artificial Sequence
  anti-mCD8 YTS 169 cDb nucleic acid sequence
tctagagccgccaccatggaaaccgacaccctgctgctgtgggtgctgctgctctgggtccccggaagcaccggtgaagtgaagctgcaggaaagcggcggaggcctggtgcagcccggcagaagcctgaagctgagctgtgccgccagcggcttcaacttcaacgactactggatgggctgggtccgacaggctcctggcaagggcctggaatggatcggcgagatcaacaaggacagcagcaccatcaactacacccccagcctgaaggacaagttcaccatcagcagagacaacgcccagaacaccctgtacctgcagatgagcaagctgggcagcgaggacaccgccatctactactgcgccagagccagaggcatgatggtgctgatcatcccccactacttcgactactggggccagggcgtgatggtcaccgtgtccagcggcggcggaggatctgacatcgtgctgacccagagccccgctatggccatgagccctggcgagagaatcacaatcagctgcagagccagcgagagcgtgtccaccagaatgcactggtatcagcagaagcccggccagcagcccaagctgctgatctacggcgccagcaacctggaatccggcgtgccagccagattcagcggcagcggctccggcaccgacttcaccctgaccatcgaccccgtggaagccaacgacaccgccacctatttctgccagcagtcttggtacgacccctggaccttcggtggaggcaccaagctggaactgaaggcggccgcaggctgcggaggccaccaccaccatcaccattgagaattc

SEQ ID NO: 41 - protein, 273 aa, Artificial Sequence
  anti-mCD8 YTS 169 cDb amino acid sequence
SRAATMETDTLLLWVLLLWVPGSTGEVKLQESGGGLVQPGRSLKLSCAASGFNFNDYWMGWVRQAPGKGLEWIGEINKDSSTINYTPSLKDKFTISRDNAQNTLYLQMSKLGSEDTAIYYCARARGMMVLIIPHYFDYWGQGVMVTVSSGGGGSDIVLTQSPAMAMSPGERITISCRASESVSTRMHWYQQKPGQQPKLLIYGASNLESGVPARFSGSGSGTDFTLTIDPVEANDTATYFCQQSWYDPWTFGGGTKLELKAAAGCGGHHHHHH

SEQ ID NO: 42 - DNA, 1272 bp, Artificial Sequence
  anti-mCD8 2.43 Mb nucleic acid sequence
tctagagccgccaccatggaaaccgacaccctgctgctgtgggtgctgctgctctgggtgcccggcagcaccggtgaagtgcagctggtggaaagcggcggaggcctggtgcagcccggcagaagcctgaagctgagctgtgccgccagcggcttcaccttcagcaactactacatggcttgggtgcgccaggcccccaccaagggactggaatgggtggcctacatcaacaccggcggaggcaccacctactacagagacagcgtgaagggcagattcaccatcagcagggacgacgccaagagcaccctgtacctgcagatggacagcctgagaagcgaggacaccgctacctactactgcaccaccgccatcggctactacttcgactactggggccagggcgtgatggtgacagtgtccagcggcagcacctctggcggcggatctggcggaggaagcggaggcggcggaagcagcgacatccagctgacacagagccccgccagcctgagcgcctctctgggcgagacagtgtctatcgagtgcctggccagcgaggacatctacagctacctggcctggtatcagcagaagcccggcaagagcccccaggtgctgatctacgccgccaacagactgcaggacggcgtgcccagcagattcagcggctctggcagcggcacccagtacagcctgaagatcagcggcatgcagcccgaggacgagggcgactacttctgtctgcagggcagcaagttcccctacaccttcggcgctggcaccaagctggaactgaagctcgagcccagaggccccaccatcaagccctgccctccctgcaagtgccctgcccctaacctgctgggcggacctggatctgtgcgggctccccaggtgtacgtgctgcccccacccgaggaagagatgaccaagaaacaggtgacactgacctgcatggtgacagacttcatgcccgaggatatctacgtggaatggaccaacaacggcaagaccgagctgaactacaagaacaccgagcccgtgctggacagcgacggcagctacttcatgtacagcaagctgcgggtggaaaagaaaaactgggtggaacggaacagctacagctgcagcgtggtgcacgagggcctgcacaactaccacaccaccaagagcttcagcaggacccccggcaaagcggccgcggccggccatctgcctgaaacaggggccggccaccaccaccatcaccatgcggccgcttgagaattc

SEQ ID NO: 43 - protein, 421 aa, Artificial Sequence
  anti-mCD8 2.43 Mb amino acid sequence
SRAATMETDTLLLWVLLLWVPGSTGEVQLVESGGGLVQPGRSLKLSCAASGFTFSNYYMAWVRQAPTKGLEWVAYINTGGGTTYYRDSVKGRFTISRDDAKSTLYLQMDSLRSEDTATYYCTTAIGYYFDYWGQGVMVTVSSGSTSGGGSGGGSGGGGSSDIQLTQSPASLSASLGETVSIECLASEDIYSYLAWYQQKPGKSPQVLIYAANRLQDGVPSRFSGSGSGTQYSLKISGMQPEDEGDYFCLQGSKFPYTFGAGTKLELKLEPRGPTIKPCPPCKCPAPNLLGGPGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNYHTTKSFSRTPGKAAAAGHLPETGAGHHHHHHAAA

SEQ ID NO: 44 - DNA, 810 bp, Artificial Sequence
  anti-mCD8 2.43 cDb nucleic acid sequence
tctagagccgccaccatggaaaccgacaccctgctgctgtgggtgctgctgctctgggtgcccggcagcaccggtgaagtgcagctggtggaaagcggcggaggcctggtgcagcccggcagaagcctgaagctgagctgtgccgccagcggcttcaccttcagcaactactacatggcttgggtgcgccaggcccccaccaagggactggaatgggtggcctacatcaacaccggcggaggcaccacctactacagagacagcgtgaagggcagattcaccatcagcagggacgacgccaagagcaccctgtacctgcagatggacagcctgagaagcgaggacaccgctacctactactgcaccaccgccatcggctactacttcgactactggggccagggcgtgatggtgacagtgtccagcggcggcggaggaagcgacatccagctgacacagagccccgccagcctgagcgcctctctgggcgagacagtgtctatcgagtgcctggccagcgaggacatctacagctacctggcctggtatcagcagaagcccggcaagagcccccaggtgctgatctacgccgccaacagactgcaggacggcgtgcccagcagattcagcggctctggcagcggcacccagtacagcctgaagatcagcggcatgcagcccgaggacgagggcgactacttctgtctgcagggcagcaagttcccctacaccttcggcgctggcaccaagctggaactgaaggcggccgcaggctgcggaggccaccaccaccatcaccattgagaattc

SEQ ID NO: 45 - protein, 267 aa, Artificial Sequence
  anti-mCD8 2.43 cDb amino acid sequence
SRAATMETDTLLLWVLLLWVPGSTGEVQLVESGGGLVQPGRSLKLSCAASGFTFSNYYMAWVRQAPTKGLEWVAYINTGGGTTYYRDSVKGRFTISRDDAKSTLYLQMDSLRSEDTATYYCTTAIGYYFDYWGQGVMVTVSSGGGGSDIQLTQSPASLSASLGETVSIECLASEDIYSYLAWYQQKPGKSPQVLIYAANRLQDGVPSRFSGSGSGTQYSLKISGMQPEDEGDYFCLQGSKFPYTFGAGTKLELKAAAGCGGHHHHHH

SEQ ID NO: 46 - DNA, 1269 bp, Artificial Sequence
  anti-mCD8 YTS 156 Mb nucleic acid sequence
tctagagccgccaccatggaaaccgacaccctgctgctgtgggtgctgctgctctgggtccccggaagcaccggtgaagtgaagctgcaggaaagcggccccagcctggtgcagcctagccagaccctgagcctgacctgcagcgtgtccggcttcagcctgatcagcgacagcgtgcactgggtccgacagcctcccggcaagggcctggaatggatgggcggcatctgggccgacggctccaccgactacaacagcgccctgaagtccagactgagcatcagcagagacaccagcaagagccagggcttcctgaagatgaacagcctgcagaccgacgacaccgccatctatttctgcaccagcaaccgcgagagctactacttcgactactggggccagggcgtgatggtcaccgtgtccagcggctctaccagcggcggaggctctggcggaggatctggtggcggcggaagcagcgacatccagatgacccagagccctgccagcctgagcgccagcctgggcgacaaagtgaccatcacctgtcaggccagccagaacatcgacaagtatatcgcctggtatcagcagaagcctggcaaggcccccagacagctgatccactacaccagcacactggtgtccggcacccccagcagattcagcggcagcggctccggcagagactacagcttcagcatcagctccgtggaaagcgaggatatcgccagctactactgcctgcagtacgacaccctgtacaccttcggcgctggcaccaagctggaactgaagctcgagcccagaggccccaccatcaagccctgccctccctgcaagtgccctgcccctaacctgctgggcggacctggatctgtgcgggctccccaggtgtacgtgctgcccccacccgaggaagagatgaccaagaaacaggtgacactgacctgcatggtgacagacttcatgcccgaggatatctacgtggaatggaccaacaacggcaagaccgagctgaactacaagaacaccgagcccgtgctggacagcgacggcagctacttcatgtacagcaagctgcgggtggaaaagaaaaactgggtggaacggaacagctacagctgcagcgtggtgcacgagggcctgcacaactaccacaccaccaagagcttcagcaggacccccggcaaagcggccgcggccggccatctgcctgaaacaggggccggccaccaccaccatcaccatgcggccgcttgagaattc

SEQ ID NO: 47 - protein, 420 aa, Artificial Sequence
  anti-mCD8 YTS 156 Mb amino acid sequence
SRAATMETDTLLLWVLLLWVPGSTGEVKLQESGPSLVQPSQTLSLTCSVSGFSLISDSVHWVRQPPGKGLEWMGGIWADGSTDYNSALKSRLSISRDTSKSQGFLKMNSLQTDDTAIYFCTSNRESYYFDYWGQGVMVTVSSGSTSGGGSGGGSGGGGSSDIQMTQSPASLSASLGDKVTITCQASQNIDKYIAWYQQKPGKAPRQLIHYTSTLVSGTPSRFSGSGSGRDYSFSISSVESEDIASYYCLQYDTLYTFGAGTKLELKLEPRGPTIKPCPPCKCPAPNLLGGPGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNYHTTKSFSRTPGKAAAAGHLPETGAGHHHHHHAAA

SEQ ID NO: 48 - DNA, 807 bp, Artificial Sequence
  anti-mCD8 YTS 156 cDb nucleic acid sequence
tctagagccgccaccatggaaaccgacaccctgctgctgtgggtgctgctgctctgggtccccggaagcaccggtgaagtgaagctgcaggaaagcggccccagcctggtgcagcctagccagaccctgagcctgacctgcagcgtgtccggcttcagcctgatcagcgacagcgtgcactgggtccgacagcctcccggcaagggcctggaatggatgggcggcatctgggccgacggctccaccgactacaacagcgccctgaagtccagactgagcatcagcagagacaccagcaagagccagggcttcctgaagatgaacagcctgcagaccgacgacaccgccatctatttctgcaccagcaaccgcgagagctactacttcgactactggggccagggcgtgatggtcaccgtgtccagcggcggcggaggctctgacatccagatgacccagagccctgccagcctgagcgccagcctgggcgacaaagtgaccatcacctgtcaggccagccagaacatcgacaagtatatcgcctggtatcagcagaagcctggcaaggcccccagacagctgatccactacaccagcacactggtgtccggcacccccagcagattcagcggcagcggctccggcagagactacagcttcagcatcagctccgtggaaagcgaggatatcgccagctactactgcctgcagtacgacaccctgtacaccttcggcgctggcaccaagctggaactgaaggcggccgcaggctgcggaggccaccaccaccatcaccattgagaattc

SEQ ID NO: 49 - protein, 266 aa, Artificial Sequence
  anti-mCD8 YTS 156 cDb amino acid sequence
SRAATMETDTLLLWVLLLWVPGSTGEVKLQESGPSLVQPSQTLSLTCSVSGFSLISDSVHWVRQPPGKGLEWMGGIWADGSTDYNSALKSRLSISRDTSKSQGFLKMNSLQTDDTAIYFCTSNRESYYFDYWGQGVMVTVSSGGGGSDIQMTQSPASLSASLGDKVTITCQASQNIDKYIAWYQQKPGKAPRQLIHYTSTLVSGTPSRFSGSGSGRDYSFSISSVESEDIASYYCLQYDTLYTFGAGTKLELKAAAGCGGHHHHHH

SEQ ID NO: 50 - protein, 4 aa, Artificial Sequence
  peptide sequence
GGGS

SEQ ID NO: 51 - protein, 8 aa, Artificial Sequence
  peptide sequence
GGGSGGGS

SEQ ID NO: 52 - protein, 18 aa, Artificial Sequence
  peptide sequence
GSTSGGGSGGGSGGGGSS

SEQ ID NO: 53 - protein, 13 aa, Artificial Sequence
  C-terminal amino acid sequence
AGSADDARKDAGS

SEQ ID NO: 54 - protein, 18 aa, Artificial Sequence
  N-terminal amino acid sequence
GSKDDARKDDARKDGSSA

(19) United States
(12) Patent Application Publication
(10) Pub. No.: US 2015/0191543 A1
(43) Pub. Date: Jul. 9, 2015

(54) ENGINEERED ANTIBODY FRAGMENTS FOR TARGETING AND IMAGING CD8 EXPRESSION IN VIVO

(71) Applicant: THE REGENTS OF THE UNIVERSITY OF CALIFORNIA, Oakland, CA (US)

(72) Inventors: Anna M. Wu, Sherman Oaks, CA (US); Richard Tavaré, Manhattan Beach, CA (US); Tove Olafsen, Reseda, CA (US)

(21) Appl. No.: 14/419,225

(22) PCT Filed: Aug. 6, 2013
(86) PCT No.: PCT/US2013/053862

§ 371 Date: Feb. 2, 2015

Related U.S. Application Data
(60) Provisional application No. 61/680,165, filed on Aug. 6, 2012.

(51) Int. Cl. C07K 16/28; A61K 51/10

(52) U.S. Cl. CPC C07K 16/2815; A61K 51/1027; C07K 2317/565; C07K 2317/622; C07K 2317/626; C07K 2317/24; A61K 2039/505

(57) ABSTRACT

Disclosed herein, the parental antibodies from the hybridomas YTS 169.4.2.1 (YTS169) and 2.43 were engineered into minibody and diabody fragments. Both the YTS and 2.43 antibodies bind mCD8+. However, they differ in that the YTS 169 antibodies bind both Lyt2.1 and Lyt2.2 while the 2.43 antibodies bind an epitope that is Lyt2.2 specific. These novel minibodies and diabodies retained their antigen specificity as shown by flow cytometry and ImmunoPET imaging. Most importantly, both the 2.43 and YTS169 minibodies and diabodies produced high contrast ImmunoPET images of CD8+ lymphoid organs at only four hours post-injection.

### CROSS REFERENCE TO RELATED APPLICATIONS

This application is a 371 National Phase of PCT/US2013/053862 filed Aug. 6, 2013 which claims the benefit under 35 U.S.C. §119(e) to U.S. Application No. 61/680,165 filed Aug. 6, 2012, the disclosures of which are incorporated by reference in their entireties.

### STATEMENT AS TO GOVERNMENT SUPPORT

This invention was made with Government support under Grant Nos. CA016042, CA092131, and CA098010, awarded by the National Institutes of Health. The Government has certain rights in the invention.

### FIELD OF THE INVENTION

This invention relates to novel antibody-based agents for imaging in vivo CD8 expression in mouse models of disease and novel, non-invasive and detection and quantification of CD8⁺ T cells in vivo.

### BACKGROUND

Non-invasive detection of specific biomarkers of disease can provide crucial information for diagnosis, prognosis, response to therapy, dosage for radioimmunotherapy and targeted therapy selection. Specifically, the non-invasive detection and quantification of CD8⁺ T cells in vivo is important for both the detection and staging of CD8⁺ lymphomas and the monitoring of successful cancer immunotherapies. Furthermore, the detection and quantification of CD8⁺ T cells in vivo is useful for monitoring adoptive cell transfer or antibody-based immunotherapeutics.

The rapid increase of therapeutic antibodies approved by the FDA and currently in Phase I-III clinical trials for oncological, autoimmune diseases, and inflammatory diseases, among other conditions, has resulted from advances in antibody engineering, protein conjugation chemistry, and biomarker identification (Pillay V. et al., Antibodies in oncology. N Biotechnol. September 2011; 28(5):518-529; Rothe A. et al., Recombinant proteins in rheumatology—recent advances. N Biotechnol. September 2011; 28(5):502-510; Wu A. M. et al., Arming antibodies: prospects and challenges for immunoconjugates. Nat Biotechnol. September 2005; 23(9):1137-1146). Concurrently, immuno-positron emission tomography imaging (ImmunoPET) agents using intact antibodies has shown promise in both preclinical and clinical detection of cancer in vivo (Knowles S. M. et al., Advances in immuno-positron emission tomography: antibodies for molecular imaging in oncology. J Clin Oncol. Nov. 1 2012; 30(31):3884-3892).

While much progress has been made in the ImmunoPET detection of oncological markers (Knowles S. M. et al., Advances in immuno-positron emission tomography: antibodies for molecular imaging in oncology. J Clin Oncol. Nov. 1 2012; 30(31):3884-3892), the non-invasive monitoring of immune cells in the fields of infection, cancer, and autoimmunity remains challenging. Practiced methods for lymphocyte detection include isolation of cells from the peripheral blood or, less commonly, the tissue of interest. However, the invasive tissue sampling methods are prone to error and do not provide dynamic information that reflect the number, location and movement of lymphoid cells. Therefore, problems still exist for the evaluation of immunotherapy protocols due to the lack of effective methods to monitor the extent and duration of the therapy.

Current methods to monitor immune cells non-invasively using emission tomography include direct cell labeling, reporter genes, and small molecule PET tracers. The ex vivo direct labeling of immune cells with PET (positron emission tomography) or SPECT (single positron emission tomography) probes before subsequent re-injection and imaging has enabled in vivo trafficking of lymphocytes (Matsui K. et al., Quantitation and visualization of tumor-specific T cells in the secondary lymphoid organs during and after tumor elimination by PET. Nucl Med Biol. November 2004; 31(8):1021-1031; Pittet M. J. et al., In vivo imaging of T cell delivery to tumors after adoptive transfer therapy. Proc Natl Acad Sci USA. Jul. 24 2007; 104(30):12457-12461). However, this method has inherent limitations such as radioisotope half-life and cell division in vivo that leads to probe dilution.

Reporter gene imaging of the adaptive immune response, whereby cells are transfected with a PET reporter gene that encodes a protein that is specifically targeted via a radio-labeled reporter probe (Nair-Gill E. D. et al., Non-invasive imaging of adaptive immunity using positron emission tomography. Immunol Rev. February 2008; 221:214-228; Yaghoubi S. S. et al., Positron emission tomography reporter genes and reporter probes: gene and cell therapy applications. Theranostics. 2012; 2(4):374-391), has been used to image adoptive cell transfer of transduced T cell receptor engineered lymphocytes (Koya R. C. et al., Kinetic phases of distribution and tumor targeting by T cell receptor engineered lymphocytes inducing robust antitumor responses. Proc Natl Acad Sci USA. Aug. 10 2010; 107(32):14286-14291). Similarly, a PET (positron emission tomography) and SPECT (single positron emission tomography) reporter gene have been used for the tracking of regulatory T cells (harif-Paghaleh E. et al., In vivo SPECT reporter gene imaging of regulatory T cells. PLoS One. 2011; 6(10):e25857). Accordingly, reporter gene imaging allows for longitudinal tracking of cells but the technology is limited because it relies on the ex vivo transfection of cells and, for clinical translation, the development of non-immunogenic PET reporter proteins (Yaghoubi S. S. et al., Positron emission tomography reporter genes and reporter probes: gene and cell therapy applications. Theranostics. 2012; 2(4):374-391).

In another approach, small molecule PET probes targeting metabolic pathways, including [(18)F]-Fluorodeoxyglucose ([(18)F]-FDG), [(18)F]-fluorothymidine ([(18)F]-FLT), and [(18)F]-1-(2-doexy-2-fluoro-arabinofuranosyl)-cytosine ([(18)F]-FAC), have the potential to non-invasively monitor diverse cell types of both innate and adaptive immunity (Laing R. E. et al., Visualizing cancer and immune cell function with metabolic positron emission tomography. Curr Opin Genet Dev. February 2010; 20(1):100-105). Clinically, [¹⁸F]FDG-PET has been used to evaluate inflammation in graft-versus-host disease (Stelljes M. et al., Clinical molecular imaging in intestinal graft-versus-host disease: mapping of disease activity, prediction, and monitoring of treatment efficiency by positron emission tomography. Blood. Mar. 1 2008; 111(5):2909-2918), atherosclerosis (Rudd J. H. et al., Inflammation imaging in atherosclerosis. Arterioscler Thromb Vasc Biol. July 2009; 29(7):1009-1016), infectious diseases (Basu S. et al., Positron emission tomography as a diagnostic tool in infection: present role and future possibilities. Semin Nucl Med. January 2009; 39(1):36-51), and rheumatiod arthritis (Elzinga E. H. et al., 2-Deoxy-2-[F-18]fluoro-D-glucose joint uptake on positron emission tomography images: rheumatoid arthritis versus osteoarthritis. Mol Imaging Biol. November-December 2007; 9(6):357-360).

However, in the context of immune cell detection in oncology, false positives can occur when using small molecule PET probes targeting metabolic pathways. These false positives are often the result of the utilization of glycolysis in both cancer and immune cells, both innate and adaptive, in the tumor itself or the draining lymph nodes (Juweid M. E. et al., Positron-emission tomography and assessment of cancer therapy. N Engl J Med. Feb. 2 2006; 354(5):496-507; Mamede M. et al., Differential uptake of (18)F-fluorodeoxyglucose by experimental tumors xenografted into immunocompetent and immunodeficient mice and the effect of immunomodification. Neoplasia. March-April 2003; 5(2):179-183).

For example, [(18)F]-FLT-PET accumulates in highly proliferative tissues and most research has been focused on cancer detection. [(18)F]-FLT-PET suffers from high uptake in proliferating bone marrow therefore limiting detection of lesions in bone. [(18)F]-FLT-PET was recently used to detect antigen specific immune responses in melanoma patients with lymph node metastases using dendritic cell therapy (Aarntzen E. H. et al., Early identification of antigen-specific immune responses in vivo by [18F]-labeled 3′-fluoro-3′-deoxy-thymidine ([18F]FLT) PET imaging. Proc Natl Acad Sci USA. Nov. 8 2011; 108(45):18396-1839).

Furthermore, [(18)F]-FAC)-PET can distinguish between innate and adaptive immune cells due to the upregulation of deoxycytidine kinase in proliferating T cells, but the uptake in a MSV/MuLV induced sarcoma model is limited to proliferating T cells in the draining lymph node (Nair-Gill E. et al., PET probes for distinct metabolic pathways have different cell specificities during immune responses in mice. J Clin Invest. June 2010; 120(6):2005-2015). Therefore, like the other metabolic tracers [(18)F]-FDG-PET and [(18)F]-FLT-PET, [(18)F]-FAC uptake due to activation-induced T cell proliferation is restricted to the draining lymph nodes and was unable to image tumor T cell infiltration (Nair-Gill E. et al., PET probes for distinct metabolic pathways have different cell specificities during immune responses in mice. J Clin Invest. June 2010; 120(6):2005-2015). Accordingly, there is a need for technology that is not proliferation-dependent that can be used for immunotherapeutic diagnosis.

This makes immunoPET imaging using antibody fragments targeting specific immune cell antigens critical for immunotherapeutic diagnosis because the binding is not proliferation-dependent. Specifically, antibody fragments targeting CD8+ cells is particularly important because CD8 is present on all cytotoxic T cells and binding is not proliferation-dependent. Furthermore, antibodies are particularly important because unlike the small molecule PET tracers, antibodies have the ability distinguish between immune cell subtypes, such as cytotoxic T cells (CD8), helper T cells (CD4) and natural killer cells (CD16).

Intact antibodies have relatively long serum half-lives (1-3 weeks) when compared to their engineered counterparts, such as the diabody and minibody, that have terminal half-lives that range from 2-5 hours and 8-12 hours, respectively (Olafsen T. et al., Antibody vectors for imaging. Semin Nucl Med. May 2010; 40(3):167-181). While decreasing the total uptake in tumors, the rapid clearance of engineered antibody fragments allows for much higher tumor-to-background images at much earlier times post-injection. This allows for the potential of not only same-day imaging but also reduces overall radiation dose. Furthermore, the majority of these engineered fragments are biologically inert as they lack Fc effector function capabilities. Accordingly, there is a need to develop antibody fragments that can be used to obtain rapid, high-contrast ImmunoPET.

Historically, the development of immunoPET agents has focused on over-expressed tumor cell surface antigens or angiogenic targets, with the exception of the B cell antigen CD20. ImmunoPET agents that can be used to obtain rapid, high contrast images for the detection of CD8 expression in vivo have not be previously described. Described herein are anti-murine CD8 antibodies, specifically minibodies (Mbs) and diabodies (Dbs), for ImmunoPET imaging and detection of CD8 expression in vivo.

### BRIEF SUMMARY OF THE INVENTION

In the first aspect of this application, this invention comprises an antibody that binds to CD8 comprising the V_{H} domain of any of SEQ ID NOs.: 7, 15, or 23, and the V_{L} domain of any of SEQ ID NOs.: 11, 19, or 27. In certain embodiments the antibody comprises the V_{H} domain of SEQ ID NO.: 7 and the V_{L} domain of any of SEQ ID NO.: 11. In certain embodiments the antibody comprises the V_{H} domain of SEQ ID NO.: 15 and the V_{L} domain of any of SEQ ID NO.: 19. In certain embodiments the antibody comprises the V_{H} domain of SEQ ID NO.: 23 and the V_{L} domain of any of SEQ ID NO.: 27.

In certain embodiments, this invention comprises an antibody that binds to CD8 comprising a heavy chain CDR1 of any of SEQ ID NOs.: 8, 16, or 24, a heavy chain CDR2 of any of SEQ ID NOs.: 9, 17, or 25, a heavy chain CDR3 of any of SEQ ID NOs.: 10, 18, or 26, a light chain CDR1 of any of SEQ ID NOs.: 12, 20, or 28, a light chain CDR2 of any of SEQ ID NOs.: 13, 21, or 29, and a light chain CDR3 of any of SEQ ID NOs.: 14, 22, or 30.

In specific embodiments the antibody comprises a heavy chain CDR1 of SEQ ID NO.: 8, a heavy chain CDR2 of SEQ ID NO.: 9, a heavy chain CDR3 of SEQ ID NO.: 10, a light chain CDR1 of SEQ ID NO.: 12, a light chain CDR2 of SEQ ID NO.: 13, and a light chain CDR3 of SEQ ID NO.: 14. In specific embodiments the antibody comprises a heavy chain CDR1 of SEQ ID NO.: 16, a heavy chain CDR2 of SEQ ID NO.: 17, a heavy chain CDR3 of SEQ ID NO.: 18, a light chain CDR1 of SEQ ID NO.: 20, a light chain CDR2 of SEQ ID NO.: 21, and a light chain CDR3 of SEQ ID NO.: 22. In specific embodiments the antibody comprises a heavy chain CDR1 of SEQ ID NO.: 24, a heavy chain CDR2 of SEQ ID NO.: 25, a heavy chain CDR3 of SEQ ID NO.: 26, a light chain CDR1 of SEQ ID NO.: 28, a light chain CDR2 of SEQ ID NO.: 29, and a light chain CDR3 of SEQ ID NO.: 30.

In certain embodiments, the antibody is selected from the group consisting of scFv, scFv dimer (diabody), scFv-C_{H}3 dimer (minibody) and scFv-Fc. In certain embodiments, the antibody is a diabody. In certain embodiments, the antibody is a minibody.

In certain embodiments, the scFv dimer comprises two scFv monomers joined by a linker. In certain embodiments, the linker is a peptide sequence. In certain embodiments, the linker is selected from the group consisting of GGGS (SEQ ID NO: 50), GGGSGGGS (SEQ ID NO: 51), and GSTSGGGSGGGSGGGGSS (SEQ ID NO: 52).

In certain embodiments, the antibody is a humanized antibody fragment which binds to CD8. In certain embodiments, the antibody is a chimeric antibody.

In certain embodiments, the antibody is linked to a detectable moiety. In certain embodiments, the detectable moiety is seleted from the group consisting of a radionuclide, a nanoparticle, a fluorescent dye, a fluorescent marker, and an enzyme. In certain embodiments, the detectable moity is a radionuclide. In certain embodiments, the radionuclide is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, ¹⁸F, ¹²⁴I, ¹²⁵I, ¹³¹I, ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁹⁰Y, ¹¹¹In, ⁹⁹Tc, and ²⁰¹Tl. In certain embodiments, the detetable moity can be imaged in vivo using a system selected from the group consisting of MRI, SPECT, PET, and planar gamma camera imaging.

In the second aspect of this application, this invention comprises a method of diagnosing a CD8 mediated disease. In certain embodiments, the method comprises the steps of a) administering to a subject an antibody comprising a light chain CDR1 of any of SEQ ID NOs.: 12, 20, or 28; a light chain CDR2 of any of SEQ ID NOs.: 13, 21, or 29; a light chain CDR3 of any of SEQ ID NOs.: 14, 22, or 30; a heavy chain CDR1 of any of SEQ ID NOs.: 8, 16, or 24; a heavy chain CDR2 of any of SEQ ID NOs.: 9, 17, or 25; and a heavy chain CDR3 of any of SEQ ID NOs.: 10, 18, or 26, which antibody specifically binds to the CD8 on the surface of cells, to a subject; b) determining the expression of the CD8 protein in the subject using molecular in vivo imaging; and c) determining whether or not CD8 protein is overexpressed in the subject using molecular in vivo imaging. In certain embodiments, the overexpression of CD8 indicates a CD8 mediated disease.

In certain embodiments of the method of diagnosis described herein, the heavy chain CDR1 has the sequence comprising the amino acid sequence of SEQ ID NO.: 8, the heavy chain CDR2 has the sequence comprising the amino acid sequence of SEQ ID NO.: 9, the heavy chain CDR3 has the sequence comprising the amino acid sequence of SEQ ID NO.: 10, the light chain CDR1 has the sequence comprising the amino acid sequence of SEQ ID NO.: 12, the light chain CDR2 has the sequence comprising the amino acid sequence of SEQ ID NO.: 13, and the light chain CDR3 has the sequence comprising the amino acid sequence of SEQ ID NO.: 14.

In certain embodiments of the method of diagnosis described herein, the heavy chain CDR1 has the sequence comprising the amino acid sequence of SEQ ID NO.: 16, the heavy chain CDR2 has the sequence comprising the amino acid sequence of SEQ ID NO.: 17, the heavy chain CDR3 has the sequence comprising the amino acid sequence of SEQ ID NO.: 18, the light chain CDR1 has the sequence comprising the amino acid sequence of SEQ ID NO.: 20, the light chain CDR2 has the sequence comprising the amino acid sequence of SEQ ID NO.: 21, and the light chain CDR3 has the sequence comprising the amino acid sequence of SEQ ID NO.: 22.

In certain embodiments of the method of diagnosis described herein, the heavy chain CDR1 has the sequence comprising the amino acid sequence of SEQ ID NO.: 24, the heavy chain CDR2 has the sequence comprising the amino acid sequence of SEQ ID NO.: 25, the heavy chain CDR3 has the sequence comprising the amino acid sequence of SEQ ID NO.: 26, the light chain CDR1 has the sequence comprising the amino acid sequence of SEQ ID NO.: 28, the light chain CDR2 has the sequence comprising the amino acid sequence of SEQ ID NO.: 29, and the light chain CDR3 has the sequence comprising the amino acid sequence of SEQ ID NO.: 30.

In certain embodiments of the method of diagnosis described herein, the CD8 mediated disease is cancer. In certain embodiments of the method of diagnosis described herein, the CD8 mediated disease is an autoimmune disease.

In certain embodiments of the method of diagnosis described herein, the antibody is selected from the group consisting of scFv, scFv dimer (diabody), scFv-C_{H}3 dimer (minibody) and scFv-Fc. In certain embodiments of the method of diagnosis described herein, the antibody is a diabody. In certain embodiments of the method of diagnosis described herein, the antibody is a minibody.

In certain embodiments of the method of diagnosis described herein, the scFv dimer comprises two scFv monomers joined by a linker. In certain embodiments of the method of diagnosis described herein, the linker is a peptide sequence. In certain embodiments of the method of diagnosis described herein, the linker is selected from the group consisting of GGGS (SEQ ID NO: 50), GGGSGGGS (SEQ ID NO: 51), and GSTSGGGSGGGSGGGGSS (SEQ ID NO: 52).

In certain embodiments of the method of diagnosis described herein, the antibody is a humanized antibody fragment which binds to CD8. In certain embodiments of the method of diagnosis described herein, the antibody is a chimeric antibody.

In certain embodiments of the method of diagnosis described herein, the antibody is linked to a detectable moiety. In certain embodiments of the method of diagnosis described herein, the detectable moiety is seleted from the group consisting of a radionuclide, a nanoparticle, a fluorescent dye, a fluorescent marker, and an enzyme. In certain embodiments of the method of diagnosis described herein, the detectable moity is a radionuclide. In certain embodiments of the method of diagnosis described herein, the radionuclide is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, ¹⁸F, ¹²⁴I, ¹²⁵I, ¹³¹I, ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁹⁰Y, ¹¹¹In, ⁹⁹Tc, and ²⁰¹Tl.

In certain embodiments of the method of diagnosis described herein, the detetable moity can be imaged in vivo using a system selected from the group consisting of MRI, SPECT, PET, and planar gamma camera imaging.

In the third aspect of this application, this invention comprises a method of providing the prognosis for a CD8 mediated disease. In certain embodiments, this method comprises the steps of a) administering to a subject an antibody comprising a light chain CDR1 of any of SEQ ID NOs.: 12, 20, or 28; a light chain CDR2 of any of SEQ ID NOs.: 13, 21, or 29; a light chain CDR3 of any of SEQ ID NOs.: 14, 22, or 30; a heavy chain CDR1 of any of SEQ ID NOs.: 8, 16, or 24; a heavy chain CDR2 of any of SEQ ID NOs.: 9, 17, or 25; and a heavy chain CDR3 of any of SEQ ID NOs.: 10, 18, or 26, which antibody specifically binds to the CD8 on the surface of cells, to a subject; b) determining the expression of the CD8 protein in the subject using molecular in vivo imaging, and c) determining whether or not the CD8 protein is overexpressed in the subject using molecular in vivo imaging. In certain embodiments, the overexpression of CD8 indicates a CD8 mediated disease. In certain embodiments, the degree of overexpression of CD8 indicates the prognosis for the CD8 mediated disease. In certain embodiments, the greater the expression the poorer the prognosis.

In certain embodiments of the method of prognosis described herein, the heavy chain CDR1 has the sequence comprising the amino acid sequence of SEQ ID NO.: 8, the heavy chain CDR2 has the sequence comprising the amino acid sequence of SEQ ID NO.: 9, the heavy chain CDR3 has the sequence comprising the amino acid sequence of SEQ ID NO.: 10, the light chain CDR1 has the sequence comprising the amino acid sequence of SEQ ID NO.: 12, the light chain CDR2 has the sequence comprising the amino acid sequence of SEQ ID NO.: 13, and the light chain CDR3 has the sequence comprising the amino acid sequence of SEQ ID NO.: 14.

In certain embodiments of the method of prognosis described herein, the heavy chain CDR1 has the sequence comprising the amino acid sequence of SEQ ID NO.: 16, the heavy chain CDR2 has the sequence comprising the amino acid sequence of SEQ ID NO.: 17, the heavy chain CDR3 has the sequence comprising the amino acid sequence of SEQ ID NO.: 18, the light chain CDR1 has the sequence comprising the amino acid sequence of SEQ ID NO.: 20, the light chain CDR2 has the sequence comprising the amino acid sequence of SEQ ID NO.: 21, and the light chain CDR3 has the sequence comprising the amino acid sequence of SEQ ID NO.: 22.

In certain embodiments of the method of prognosis described herein, the heavy chain CDR1 has the sequence comprising the amino acid sequence of SEQ ID NO.: 24, the heavy chain CDR2 has the sequence comprising the amino acid sequence of SEQ ID NO.: 25, the heavy chain CDR3 has the sequence comprising the amino acid sequence of SEQ ID NO.: 26, the light chain CDR1 has the sequence comprising the amino acid sequence of SEQ ID NO.: 28, the light chain CDR2 has the sequence comprising the amino acid sequence of SEQ ID NO.: 29, and the light chain CDR3 has the sequence comprising the amino acid sequence of SEQ ID NO.: 30.

In certain embodiments of the method of prognosis described herein, the CD8 mediated disease is cancer. In certain embodiments of the method of prognosis described herein, the CD8 mediated disease is an autoimmune disease.

In certain embodiments of the method of prognosis described herein, the antibody is selected from the group consisting of scFv, scFv dimer (diabody), scFv-C_{H}3 dimer (minibody) and scFv-Fc. In certain embodiments of the method of prognosis described herein, the antibody is a diabody. In certain embodiments of the method of prognosis described herein, the antibody is a minibody.

In certain embodiments of the method of prognosis described herein, the scFv dimer comprises two scFv monomers joined by a linker. In certain embodiments of the method of prognosis described herein, the linker is a peptide sequence. In certain embodiments of the method of prognosis described herein, the linker is selected from the group consisting of GGGS (SEQ ID NO: 50), GGGSGGGS (SEQ ID NO: 51), and GSTSGGGSGGGSGGGGSS (SEQ ID NO: 52).

In certain embodiments of the method of prognosis described herein, the antibody is a humanized antibody fragment which binds to CD8. In certain embodiments of the method of prognosis described herein, the antibody is a chimeric antibody. In certain embodiments of the method of prognosis described herein, the antibody is linked to a detectable moiety.

In certain embodiments of the method of prognosis described herein, the detectable moiety is seleted from the group consisting of a radionuclide, a nanoparticle, a fluorescent dye, a fluorescent marker, and an enzyme. In certain embodiments of the method of prognosis described herein, the detectable moity is a radionuclide. In certain embodiments of the method of prognosis described herein, the radionuclide is selected from the group consisting of ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, ¹⁸F, ¹²⁴I, ¹²⁵I, ¹³¹I, ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁹⁰Y, ¹¹¹In, ⁹⁹Tc, and ²⁰¹Tl.

In certain embodiments of the method of prognosis described herein, the detetable moity can be imaged in vivo using a system selected from the group consisting of MRI, SPECT, PET, and planar gamma camera imaging.

In certain embodiments of the method of prognosis described herein, the antibody is a humanized antibody.

In certain embodiments of any of the first, second, or third embodiments, the antibody comprises the amino acid sequence of SEQ ID NO.: 2. In certain embodiments of any of the first, second, or third embodiments, the antibody comprises the amino acid sequence of SEQ ID NO.: 4. In certain embodiments of any of the first, second, or third embodiments, the antibody comprises the amino acid sequence of SEQ ID NO.: 6. In certain embodiments of any of the first, second, or third embodiments, the antibody comprises the amino acid sequence of SEQ ID NO.: 39. In certain embodiments of any of the first, second, or third embodiments, the antibody comprises the amino acid sequence of SEQ ID NO.: 43. In certain embodiments of any of the first, second, or third embodiments, the antibody comprises the amino acid sequence of SEQ ID NO.: 47. In certain embodiments of any of the first, second, or third embodiments, the antibody comprises the amino acid sequence of SEQ ID NO.: 41. In certain embodiments of any of the first, second, or third embodiments, the antibody comprises the amino acid sequence of SEQ ID NO.: 45. In certain embodiments of any of the first, second, or third embodiments, the antibody comprises the amino acid sequence of SEQ ID NO.: 49.

In certain embodiments of any of the second or third embodiments, the subject is a human.

In certain embodiments of any of the second or third embodiments, the in vivo expression of CD8 is imaged four hours post-administration.

In certain embodiments of any of the second or third embodiments, the in vivo expression of CD8 is imaged less than four hours post-administration. In certain embodiments of any of the second or third embodiments, the in vivo expression of CD8 is imaged between 3 and 5 hours post-administration. In certain embodiments of any of the second or third embodiments, the in vivo expression of CD8 is imaged between 5 and 7 hours post-administration. In certain embodiments of any of the second or third embodiments, the in vivo expression of CD8 is imaged between 7 and 10 hours post-administration. In certain embodiments of any of the second or third embodiments, the in vivo expression of CD8 is imaged between 10 and 15 hours post-administration. In certain embodiments of any of the second or third embodiments, the in vivo expression of CD8 is imaged between 15 and 20 hours post-administration. In certain embodiments of any of the second or third embodiments, the in vivo expression of CD8 is imaged between 20 and 24 hours post-administration. In certain embodiments of any of the second or third embodiments, the in vivo expression of CD8 is imaged between 7 and 10 hours post-administration.

In certain embodiments of any of the second or third embodiments, the administration is intravenous administration as a bolus, intravenous administration by continuous infusion over a period of time, intramuscular administration, intraperitoneal administration, intracerobrospinal administration, subcutaneous administration, intra-articular administration, intrasynovial administration, intrathecal administration, oral administration, or inhalation administration. In certain embodiments of any of the second or third embodiments, the administration is intravenous administration.

### BRIEF DESCRIPTION OF THE DRAWINGS

FIG. 1 depicts antibody fragments reduce both blood half-life and total tumor uptake, but they allow for high signal-to-noise for molecular imaging purposes.

FIG. 2 is a review of engineering and production of novel anti-mCD8 minibodies in mouse NS0 myeloma cells. The engineered Minibody fragment contains the murine Ig kappa secretion signal (L) followed by the VH and VL domains separated by an 18 GlySer-rich amino acid linker. After the VL domain is the murine IgG2a hinge sequence followed by the murine IgG2a CH3 domain and a HisTag. After subcloning into the mammalian pEE12 expression vector, NS0 cells were electroporated and diluted for clonal expansion. Clones are screened for production and expanded for production in terminal cultures. Minibodies are then purified directly from the supernatant using NiNTA columns.

FIG. 3A-FIG. 3B depict Minibody construction and epitope specificity. FIG. 3A depicts the anti-CD8 2.43 and YTS169 minibodies contain the rat V_{H}-V_{L} separated by an 18 amino acid linker followed by the mouse IgG2a hinge, C_{H}3 and a C-terminal HexaHistidine tag. FIG. 3B shows that mouse CD8a is expressed as two isoforms, Lyt2.1 and Lyt2.2, that vary in a single amino acid and is restricted to specific mouse strains. The 2.43 Minibody binds CD8a in only Lyt2.2⁺ mouse strains while the YTS169 Minibody binds CD8a on all mouse strains.

FIG. 4A-FIG. 4C show purification of the minibodies on a NiNTA column after NSO electroporation and terminal culture of the Minibody expressing NS0 clones. The yields of the minibodies 2.43, YTS 169 and YTS 156 were 6.6, 8.9 and 0.25 mg/L, respectively. Purified protein was then run on Superdex200 size exclusion chromatography for elution profiles and compared to reference standards for the correct molecular weight. FIG. 4A shows a representative example of the UV chromatogram of the anti-mCD9 169 Minibody protein elution over time with an increase in concentration of imidazole (dotted line). FIG. 4B depicts samples from the elution that were run on SDS-PAGE for protein elution analysis. Protein eluted from 25-45 min. were combined, dialyzed against PBS, and concentrated for further use. FIG. 4C depicts the size exclusion chromatography which shows the presence of 81%˜80 kDa minibody for the 2.43 minibody and 23%˜80 kDa minibody for the YTS169 minibody.

FIG. 5A- to FIG. 5B depict flow cytometry that shows specific binding of Minibody fragments to the different murine CD8 epitopes Lyt2.1, Lyt2.2 and Lyt3. FIG. 5A shows that both YTS 169 (dark gray; Lyt2.1 and Lyt2.2 specific) and 2.43 (black; Lyt2.2 specific) minibodies showed near identical binding to BW58, a Lyt2.2-positive cell line, and a reduced binding by the YTS 156 Minibody (light gray; Lyt3 specific). FIG. 5B shows that the cell line TK-1, a Lyt2.1-positive cell line, showed binding for YTS 169 and YTS 156 minibodies, but very low binding to the 2.43 Minibody due to the Lyt2.1 specificity of the 2.43 Minibody. This binding is slightly higher than an irrelevant Minibody staining (solid grey).

FIG. 6A- to FIG. 6B show that the 2.43 Minibody retains Lyt2.2 antigen specificity. FIG. 6A depicts primary cells isolated from the peripheral blood, thymus, spleen and lymph nodes of B/6 and C3H mice are stained with PE-anti-CD4 and FITC-anti-CD8 antibody. FIG. 6B depicts primary cells isolated from the peripheral blood, thymus, spleen and lymph nodes of B/6 and C3H mice are stained with PE-anti-CD4 and Lyt2.2-specific FITC-2.43 Minibody. The lack of CD8-FITC staining in Lyt2.1⁺ C3H mice shows the 2.43 Minibody specificity for Lyt2.2⁺ B/6 mice.

FIG. 7A- to FIG. 7B show that anti-CD8 Minibody does not deplete CD8 expressing cells in vivo. B/6 mice were treated for three consecutive days with either (FIG. 7A) 330 μg of anti-CD8 depleting antibody (clone 53-6.7) injected intraperitoneally or (FIG. 7B) 250 μg of 2.43 Minibody injected intravenously. Cells were then isolated from the peripheral blood, thymus, spleen and lymph nodes for staining with anti-CD4-PE and FITC-conjugated 2.43 Minibody.

FIG. 8 depicts representative PET images of B/6, C3H and NSG SDIC mice at 4 hours post-injection of 64Cu-NOTA 2.43 Minibody. Upper panel shows the PET only image and the lower paned is PET/CT images. Abbreviations: Bn—bone; Sp—spleen; Li—liver; Bl—bladder; C.LN—cervical LNs; A.LNs—axillary LNs; E-I.LNs—external iliac LNs; I-I.LNs—internal iliac LNs; and P.PNs—popliteal LNs.

FIG. 9A- to FIG. 9B depict ImmunoPET imaging (FIG. 9A) of ⁶⁴Cu-NOTA-2.43 Minibody four hours post-injection and quantification (FIG. 9B). ImmunoPET/CT images were acquired B/6 mice four hours post-intravenous injection. The white arrows in the right panels (2 mm transversal MIPs) are used to highlight uptake in various lymph nodes and the spleen seen in the whole body 20 mm coronal MIPs shown on the left. Abbreviations: C.LN—cervical lymph nodes, A.LN—axillary lymph nodes, Li—liver, Sp—Spleen, I.LN—inguinal lymph nodes, B—bone, and P.LN—popliteal lymph nodes. Abbreviations: Bn—bone; Sp—spleen; Li—liver; Bl—bladder.

FIG. 10A- to FIG. 10B depict ImmunoPET imaging of ⁶⁴Cu-NOTA-2.43 Minibody and ⁶⁴Cu-NOTA-YTS169 Minibody shows in vivo specificity of the 2.43 Minibody to Lyt2.2⁺ mice. FIG. 10A depicts ImmunoPET imaging 4 hours post-injection of ⁶⁴Cu-NOTA-2.43 Minibody into B/6, C3H, and NOD scid gamma mice. FIG. 10B depicts ImmunoPET imaging 4 hours post-injection of ⁶⁴Cu-NOTA-YTS169 Minibody into B/6 and C3H mice. Solid white arrows in top panels (20 mm coronal MIPs) indicate where the bottom panel transversal images (2 mm MIPs) are acquired. Hollow white arrows indicate the location of the spleen.

FIG. 11A- to FIG. 11C depict ImmunoPET imaging of ⁶⁴Cu-NOTA-2.43 Minibody in antigen-blocked and antigen-depleted B/6 mice. ImmunoPET images were acquired four hours post injection of ⁶⁴Cu-NOTA-2.43 Minibody into: (FIG. 11A) wild type B/6, (FIG. 11B) B/6 mice blocked with 4 mg/kg cold minibody (bolus intravenous injection with ⁶⁴Cu-NOTA-2.43 Minibody), and (FIG. 11C) B/6 mice treated with an anti-CD8 depleting antibody. Solid white arrows in top panels (20 mm coronal MIPs) indicate where the bottom panel transversal images (2 mm MIPs) are acquired. Hollow white arrows indicate the location of the spleen.

FIG. 12 shows the purification and characterization of 2.43 and YTS169 minibodies. Both the 2.43 (black line) and YTS169 minibody (grey line) bind CD8 expressed on the Lyt2.2⁺ BW58 murine thymoma cell line (left panel). However, only the YTS169 minibody binds CD8 expressed on the Lyt2.1⁺ TK-1 murine lymphoma cell line (right panel). Solid grey is secondary antibody only.

FIG. 13A- to FIG. 13E depict the purification of the sCDαβ antigen. sCD8αβ is purified in two steps. First, NiNTA immobilized metal affinity chromatography using imidazole elution (FIG. 13A) purified the sCD8αβ and some higher molecular weight contaminants, as shown by SDS-PAGE (FIG. 13B). Secondly, size exclusion chromatography (FIG. 13C) was used to remove contaminants as shown by SDS-PAGE (FIG. 13D). Purified antigen is shown in FIG. 13E.

FIG. 14A- to FIG. 14C depict the solution phase binding of 2.43 and YTS169 minibodies to sCD8αβ demonstrated by size exclusion chromatography. The 2.43 minibody (FIG. 14A) or the YTS169 minibody (FIG. 14B) were mixed with equimolar amounts of sCD8αβ for 5 minutes before size exclusion chromatography analysis. SDS-PAGE analysis (FIG. 14C) of size exclusion chromatography elutions of 2.43 minibody alone at 25 and 27.5 min (lanes 1 and 2), sCD8αβ alone at 31 min (lane 3), and 2.43 minibody plus sCD8αβ at 22 and 25 min (lanes 4 and 5). Binding of 2.43 minibody to sCD8αβ is confirmed for both the 80 kDa minibody and the ˜160 kDa multimer in lanes 4 and 5.

FIG. 15A-to FIG. 15C depict the kinetic analysis of minibody binding to sCD8αβ antigen using SPR Biacore 3000. 2.43 (FIG. 15A) or YTS169 (FIG. 15B) minibody were immobilized using goat anti-mouse IgG Fc capture. Soluble monovalent antigen was then flowed over the chip at 100 (green), 50 (magenta), 25 (red), 12.5 (dark blue), 6.25 (light blue), and 0 (black) nM. Full kinetic analysis is shown in FIG. 15C.

FIG. 16A-to FIG. 16B show that ⁶⁴Cu-DOTA-2.43 cDb targets both the spleen and lymph nodes in antigen positive Bl/6 mice but not antigen-negative C3H mice. FIG. 16A shows the biodistribution of ⁶⁴Cu-DOTA-2.43 cDb 4 hours post-injection and the increased uptake in the lymph nodes and spleens of Bl/6 mice but not C3H mice (n=4). FIG. 16B shows ImmunoPET imaging at 4 hours post-injection and confirms lymph node and spleen specific uptake. Blood activity is shown in the heart and renal clearance is shown in the kidneys. PET images are 20 mm maximum intensity projections.

FIG. 17A-to FIG. 17B show that ⁸⁹Zr-DFO-2.43 cDb targets both the spleen and lymph nodes in antigen positive Bl/6 mice but not antigen-negative C3H mice. FIG. 16A shows the biodistribution of ⁸⁹Zr-DFO-2.43 cDb 4 hours post-injection and the increased uptake in the lymph nodes and spleens of Bl/6 mice but not C3H mice (n=4). FIG. 16B shows ImmunoPET imaging at 4 hours post-injection and confirms lymph node and spleen specific uptake. Blood activity is shown in the heart and renal clearance is shown in the kidneys. PET images are 20 mm maximum intensity projections.

### DEFINITIONS

For convenience, the meaning of certain terms and phrases used in the specification, examples, and appended claims, are provided below. The definitions provided herein are not limiting and should be read broadly by one of skill in the art.

The term “preventing a disorder” as used herein, is not intended as an absolute term. Instead, prevention, e.g., of cancer or autoimmune disease, refers to delay of onset, reduced frequency of symptoms, or reduced severity of symptoms associated with the disease/disorder. Prevention therefore refers to a broad range of prophylactic measures that will be understood by those in the art. In some circumstances, the frequency and severity of symptoms is reduced to non-pathological levels, e.g., so that the individual does not need traditional therapy. In some circumstances, the symptoms of an individual receiving the compositions of the invention are only 90, 80, 70, 60, 50, 40, 30, 20, 10, 5 or 1% as frequent or severe as symptoms experienced by an untreated individual with the disorder.

Similarly, the term “treating a disorder” is not intended to be an absolute term. In some aspects, the compositions of the invention seek to reduce symptoms of the disease/disorder. In some circumstances, treatment with the leads to an improved prognosis or a reduction in the frequency or severity of symptoms.

As used herein, the term “an individual in need of treatment or prevention” refers to an individual that has been diagnosed with a disease that is caused by or affected by CD8 expression. Individuals in need of treatment also include those that have suffered an injury, disease, or surgical procedure, or individuals otherwise impaired with a genetic predisposition to CD8-related diseases. Such individuals can be any mammal, e.g., human, dog, cat, horse, pig, sheep, bovine, mouse, rat, rabbit, or primate.

As used herein, the terms “CD8-related disease” and “disease related to CD8 expression” and “disease affected by CD8 expression” and “CD8 mediated disease” refer to all diseases and disorders that are characterized by abnormal CD8 expression. Such diseases and disorders include, but are not limited to, autoimmune diseases (e.g., Addison's disease, Celiac disease, dermatomyositis, Graves disease, Hashimoto's thyroiditis, multiple sclerosis, myasthenia gravis, pernicious anemia, reactive arthritis, rheumatoid arthritis, Sjogren syndrome, systemic lupus erythematosus, type I diabetes, Crohn's disease, irritable bowel disease, autoimmune hemolytic anemia, bullous pemphigoid, Goodpasture's syndrome, pemphigus, pernicious anemia, and vasculitis), allergies, viral diseases and infections (e.g., HIV/AIDS, malaria, herpes, HCV, smallpox, the common cold, the flue, hemorrhagic fever, Epstein-Barr, Hepatitis (A, B, and C), cytomegalovirus, and human papilloma), and cancers (e.g., brain cancer, breast cancer, colon cancer, melanoma, leukemia (e.g., AML), pancreatic cancer, prostate cancer, ovarian cancer, lung cancer, and gastric cancer). Furthermore, such terms can also refer to diseases caused by viral infections (e.g., Burkitt's lymphoma, nose and throat cancers, B-cell lymphomas, liver cancer, Kaposi's sarcoma, non-Hodgkin lymphoma, and cervical cancer).

As used herein, the term “percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, the terms “identical” or percent “identity,” in the context of two or more nucleic acids or polypeptide sequences, refer to two or more sequences or subsequences that are the same sequences. Sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, designated region as measured using one of the following sequence comparison algorithms or by manual alignment and visual inspection, or across the entire sequence where not indicated. The invention provides polypeptides or polynucleotides encoding polypeptides that are substantially identical, or comprising sequences substantially identical, to the polypeptides exemplified herein (e.g., anti-CD8 antibodies). This definition also refers to the complement of a nucleotide test sequence.

As used herein, the terms “polypeptide,” “peptide,” and “protein” are used interchangeably and mean any peptide-linked chain of amino acids, regardless of length or post-translational modification. The anti-CD8 antibodies described herein can be wild-type proteins or can be variants (e.g., have substitutions, deletions, and additions). Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

All peptide sequences are written according to the generally accepted convention whereby the alpha-N-terminal amino acid residue is on the left and the alpha-C-terminal amino acid residue is on the right. As used herein, the term “N-terminus” refers to the free alpha-amino group of an amino acid in a peptide, and the term “C-terminus” refers to the free α-carboxylic acid terminus of an amino acid in a peptide. A peptide which is N-terminated with a group refers to a peptide bearing a group on the α-amino nitrogen of the N-terminal amino acid residue. An amino acid which is N-terminated with a group refers to an amino acid bearing a group on the α-amino nitrogen.

As used herein, the terms “substantially the same” and “substantially identical” can mean 65%, 70%, 75%, 80%, 85%, 90%, and preferably 91%, 92%, 93%, 94%, 95%, 96%, 97%, 98% or 99% or greater amino acid sequence identity. In other embodiments, the terms can mean at least about 75% (preferably at least about 80%, and more preferably at least about 90% or most preferably at least about 95%, of the amino-acid residues match over the defined length of the peptide sequences. In other embodiments, substantially identical sequences include sequences sharing at least 65% of the amino acid residues over their length, and at least 10% of the non-shared sequences being conservative substitutions. Sequences that are substantially identical can be identified by comparing the sequences using standard software available in sequence data banks, such as BLAST programs available from the National Cancer Center for Biotechology Information at ncbi.nlm.nih.gov.

As used herein, “biological activity” or “functional” refers to the in vivo or ex vivo activities of a compound, composition, or other mixture, or physiological responses that result upon in vivo or ex vivo administration of a compound, composition or other mixture. Biological activity thus encompasses therapeutic effects, diagnostic effects and pharmaceutical activity of such compounds, compositions, and mixtures. The term “biologically active” or “functional” when used as a modifier of the anti-CD8 antibodies described herein refers to a polypeptide that exhibits at least one activity that is characteristic of or similar to an unmodified anti-CD8 antibody. Accordingly, as used herein, an anti-CD8 antibody that retains its biological activity is functional whereby it can bind to CD8 in vivo and ex vivo.

As used herein, “pharmacokinetics” refers to the concentration of an administered compound in the serum over time. Pharmacodynamics refers to the concentration of an administered compound in target and nontarget tissues over time and the effects on the target tissue (e.g., efficacy) and the non-target tissue (e.g., toxicity). Improvements in, for example, pharmacokinetics or pharmacodynamics can be designed for a particular targeting agent or biological agent, such as by using labile linkages or by modifying the chemical nature of any linker (e.g., changing solubility, charge, and the like).

As used herein, the phrases “an effective amount” and “therapeutically effective amount” refer to an amount of an anti-CD8 antibody that is useful to support an observable change in the level of one or more biological activity characteristic of CD8, or a dose sufficient to impart a beneficial effect, e.g., an amelioration of a symptom on the recipient thereof. The specific therapeutically effective dose level for any particular subject will depend upon a variety of factors including the symptom or disorder being treated, the severity of the symptom or disorder, the activity of the specific compound, the route of administration, the rate of clearance of the compound, the duration of treatment, the drugs used in combination or coincident with the compound, the age, body weight, sex, diet, and general health of the subject, and like, as well as other factors well known in the medical arts and sciences. A therapeutically effective amount can be an amount of AA targeting compound sufficient to produce a measurable inhibition of angiogenesis in the tissue being treated, i.e., an angiogenesis-inhibiting amount. Inhibition of angiogenesis can be measured in situ by immunohistochemistry, or by other methods known to one skilled in the art. Various general considerations taken into account in determining the “therapeutically effective amount” are known to those of skill in the art and are described, e.g., in Gilman, A. G., et al., Goodman And Gilman's The Pharmacological Basis of Therapeutics, 8.sup.th ed., McGraw-Hill (1990); and Remington's Pharmaceutical Sciences, 17.sup.th ed., Mack Publishing Co., Easton, Pa. (1990).

As used herein, the term “cancer” refers to human cancers and carcinomas, sarcomas, adenocarcinomas, lymphomas, leukemias, solid and lymphoid cancers, etc. Examples of different types of cancer include, but are not limited to, prostate cancer, renal cancer (i.e., renal cell carcinoma), bladder cancer, lung cancer, breast cancer, thyroid cancer, liver cancer (i.e., hepatocarcinoma), pleural cancer, pancreatic cancer, ovarian cancer, uterine cancer, cervical cancer, testicular cancer, colon cancer, anal cancer, pancreatic cancer, bile duct cancer, gastrointestinal carcinoid tumors, esophageal cancer, gall bladder cancer, rectal cancer, appendix cancer, small intestine cancer, stomach (gastric) cancer, cancer of the central nervous system, skin cancer, choriocarcinoma; head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma. In preferred embodiments, the compositions and methods of the present invention are useful for diagnosing, imaging, proving a prognosis for, and treating prostate, bladder, or pancreatic cancer or subtypes thereof.

A “label,” “detectable moiety,” or “imaging agent” is a composition detectable by spectroscopic, photochemical, biochemical, immunochemical, chemical, radiochemical, or other physical means. A detectable moiety can be coupled either directly or indirectly to the anti-CD8 antibodies or fragments thereof described herein using methods well known in the art. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, electron-dense reagents, biotin, digoxigenin, haptens, and the like.

As described herein, compositions comprising a radionuclide coupled to an antibody or antibody fragment that recognizes CD8 are particularly useful for therapeutic, imaging, diagnostic, or prognostic purposes in a subject. The radionuclide can be directly coupled to the CD8-specific antibody or fragment, directly coupled to a linking group (e.g., a peptide linking group), or bound to a chelating agent. Methods for coupling radionuclides to proteins or linking groups or binding radionuclides to chelating agents are known to one of skill in the art. In certain instances, the compositions of the present invention comprise CD8-specific antibodies and antibody fragments conjugated to a bifunctional chelating agent that contains a radionuclide such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At and/or ²¹²Bi bound thereto. Alternatively, compositions of the present invention comprise CD8-specific antibody or fragments or linking groups conjugated thereto that are radiolabeled with a radionuclide such as ¹⁸F, ¹²⁴I, ¹²⁵I, and/or ¹³¹I. In certain other instances, the imaging compositions of the present invention comprise CD8-specific antibody or fragments conjugated to a bifunctional chelating agent that contains a radionuclide such as ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁹⁰Y, ¹¹¹In, ⁹⁹Tc, and/or ²⁰¹Tl bound thereto. Alternatively, the imaging compositions of the present invention comprise anti-CD8 antibody fragments or linking groups conjugated thereto that are radiolabeled with a radionuclide such as ¹⁸F and/or ¹³¹I. Further, Gd+3 may be conjugated to the antibody fragments of the invention for use as a contrast reagent in applications such as MRI.

A “chelating agent” refers to a compound which binds to a metal ion, such as a radionuclide, with considerable affinity and stability. In addition, the chelating agents of the present invention are bifunctional, having a metal ion chelating group at one end and a reactive functional group capable of binding to peptides, polypeptides, or proteins at the other end. Methods for conjugating bifunctional chelating agents to peptides, polypeptides, or proteins are well known in the art. Suitable bifunctional chelating agents include, but are not limited to, 1,4,7,10-tetraazacyclododecane-N,N′,N″,N′″-tetraacetic acid (DOTA), a bromoacetamidobenzyl derivative of DOTA (BAD), 1,4,8,11-tetraazacyclotetradecane-N,N′,N″,N′″-tetraacetic acid (TETA), diethylenetriaminepentaacetic acid (DTPA), the dicyclic dianhydride of diethylenetriaminepentaacetic acid (ca-DTPA), 2-(p-isothiocyanatobenzyl)diethylenetriaminepentaacetic acid (SCNBzDTPA), and 2-(p-isothiocyanatobenzyl)-5(6)-methyl-diethylenetriaminepentaacetic acid (MxDTPA) (see, e.g., Ruegg et al., Cancer Res., 50:4221-4226 (1990); DeNardo et al., Clin. Cancer Res., 4:2483-2490 (1998)). Other chelating agents include EDTA, NTA, HDTA and their phosphonate analogs such as EDTP, HDTP, and NTP (see, e.g., Pitt et al., INORGANIC CHEMISTRY IN BIOLOGY AND MEDICINE, Martell, Ed., American Chemical Society, Washington, D.C., 1980, pp. 279-312; Lindoy, THE CHEMISTRY OF MACROCYCLIC LIGAND COMPLEXES, Cambridge University Press, Cambridge, 1989; Dugas, BIOORGANIC CHEMISTRY, Springer-Verlag, New York, 1989).

The term “nanoparticle” refers to a microscopic particle whose size is measured in nanometers, e.g., a particle with at least one dimension less than about 100 nm. Nanoparticles are particularly useful as detectable moieties because they are small enough to scatter visible light rather than absorb it. For example, gold nanoparticles possess significant visible light extinction properties and appear deep red to black in solution. As a result, compositions comprising CD8-specific antibody or fragments conjugated to nanoparticles can be used for the in vivo imaging of tumors or cancerous cells in a subject. Methods for attaching polypeptides or peptides nanoparticles are well known in the art and are described in, e.g., Liu et al., Biomacromolecules, 2:362-368 (2001); Tomlinson et al., Methods Mol. Biol., 303:51-60 (2005); and Tkachenko et al., Methods Mol. Biol., 303:85-99 (2005). At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles, when conjugated to a CD8-specific antibody or fragment of the present invention, can be used as imaging agents for the in vivo detection of tumor tissue such as prostate, bladder, or pancreatic cancer tissue. Alternatively, nanoparticles can be used in therapeutic applications as drug carriers that, when conjugated to a CD8-specific antibody or fragment of the present invention, deliver chemotherapeutic agents, hormonal therapeutic agents, radiotherapeutic agents, toxins, or any other cytotoxic or anti-cancer agent known in the art to cancerous cells that overexpress CD8 on the cell surface.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “therapeutically effective amount or dose” or “therapeutically sufficient amount or dose” herein is meant a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins) and as further described herein.

The term “antibody” refers generally to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability produced by any means known in the art, such as by protease treatment or recombinantly (e.g., Fab′, F(ab′).sub.2, Fab, Fv and rIgG. See, also, Pierce Catalog and Handbook, 1994-1995 (Pierce Chemical Co., Rockford, Ill.). See, also, e.g., Kuby, J., Immunology, 3.sup.rd Ed., W. H. Freeman & Co., New York (1998). The term also refers to recombinant single chain Fv fragments (scFv). The term antibody also includes bivalent or bispecific molecules, diabodies, triabodies, and tetrabodies, minibodies, and scFv-Fc structures. See, e.g., Weiner et al. (2000) Oncogene 19: 6144; Quiocho (1993) Nature 362:293. Bivalent and bispecific molecules are described in, e.g., Kostelny et al. (1992) J Immunol. 148:1547; Pack and Pluckthun (1992) Biochemistry, 31:1579; Hollinger et al., 1993, supra; Gruber et al. (1994) J. Immunol: 5368; Zhu et al. (1997) Protein Sci 6:781; Hu et al. (1996) Cancer Res. 56:3055; Adams et al. (1993) Cancer Res. 53:4026; and McCartney, et al. (1995) Protein Eng. 8:301.

The term “antibody fragment” refers generally to any portion of an antibody that has antigen binding capability. The term includes structures that naturally occur in nature such as Fab and Fc fragments that result from protease treatment of intact antibodies or to engineered non-naturally occurring antibody structures that result from molecular biological or other manipulations that join antibody domains in configurations not normally found in nature. For example, non-native configurations of antibody domains can be derived through a variety of methods known in the art such as by construction of fusion proteins, with or without linkers, such as peptide sequences, or by covalent linkage with chemical linkers.

The term “specifically binds” means that an antibody or antibody fragment predominantly binds to a particular antigen or epitope, such as CD8.

The term “Fc” refers generally a portion of an antibody structure composed of two heavy chains that each contribute two to three constant domains, depending on the class of the antibody. It will be appreciated by the skilled artisan that an Fc can be generated by any method known in the art, such as proteolysis or by recombinant expression methods.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab.

The phrase “single chain Fv” or “scFv” refers to an antibody fragment in which the variable domains of the heavy chain and of the light chain of a traditional two chain antibody have been joined to form one chain. Typically, a linker peptide is inserted between the two chains to allow for proper folding and creation of an active binding site. Thus, the linker serves to join a VL domain to a V.H domain.

The terms “scFv-CH3 dimer” or “minibody” refer generally to an antibody fragment comprising a dimer formed by the joining of monomers comprising the structure of a scFv joined to a constant region heavy chain, such as the CH3 domain. Generally, a linker is used to join the scFv, via the VH chain, to the CH3 domain. Such linkers may advantageously contain one or more cysteine residues to allow disulfide bonding of the scFv-CH3 monomers to form a scFv-CH3 dimer or minibody.

The term “scFv-Fc” refers generally to an antibody fragment comprising a dimer formed by the joining of monomers comprising the structure of a scFv joined to an antibody Fc domain. Generally, a linker is used to join the scFv, via the VH chain, to the Fc domain. Such linkers may advantageously contain one or more cysteine residues to allow disulfide bonding to provide a scFv-Fc.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, and the like; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. It will be understood that an epitope can be either a protein, carbohydrate, lipid, nucleic acid, or small molecule entity, although protein epitopes are the most common. In the case of proteins, epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “K_{D}” and “dissociation constant” refers to the equilibrium constant and measures the propensity of the anti-CD8 antibodies disclosed herein to dissociate from their CD8 targets. The terms refer to the inverse relationship to the binding affinity of the anti-CD8 antibodies and their CD8 target. A high affinity to CD8 refers to a greater tendency to bind to CD8 relative to its dissociation from CD8 once bound. Accordingly, the smaller the K_{D}, the greater the affinity. In certain embodiments, the K_{D} of the anti-CD8 antibodies disclosed herein is approximately 5 nM, or 10 nM, or 15 nM, or 20 nM, or 25 nM, or 30 nM, or 35 nM, or 40 nM, or 45 nM, or 50 nM, or 55 nM, or 60 nM, or 65 nM, or 70 nM, or 75 nM or more. In certain embodiments, the K_{D} of the anti-CD8 antibodies disclosed herein is approximately 5 nM, or 6 nM, or 7 nM, or 8 nM, or 9 nM, or 10 nM, or 11 nM, or 12 nM, or 13 nM, or 14 nM, or 15 nM. In certain embodiments, the K_{D} of the anti-CD8 antibodies disclosed herein is approximately 15 nM, or 16 nM, or 17 nM, or 18 nM, or 19 nM, or 20 nM, or 21 nM, or 22 nM, or 23 nM, or 24 nM, or 25 nM. In certain embodiments, the K_{D} of the anti-CD8 antibodies disclosed herein is approximately 25 nM, or 26 nM, or 27 nM, or 28 nM, or 29 nM, or 30 nM, or 31 nM, or 32 nM, or 33 nM, or 34 nM, or 35 nM. In certain embodiments, the K_{D} of the anti-CD8 antibodies disclosed herein is approximately 35 nM, or 36 nM, or 37 nM, or 38 nM, or 39 nM, or 40 nM, or 41 nM, or 42 nM, or 43 nM, or 44 nM, or 45 nM. In certain embodiments, the K_{D} of the anti-CD8 antibodies disclosed herein is approximately 30 nM, or 31 nM, or 32 nM, or 33 nM, or 34 nM, or 35 nM, or 36 nM, or 37 nM, or 38 nM, or 39 nM, or 40 nM. In certain embodiments, the K_{D} of the anti-CD8 antibodies disclosed herein is approximately 33 nM or 34 nM.

Unless otherwise defined, all technical and scientific terms used herein have the same meaning as commonly understood by one of ordinary skill in the art to which this disclosure pertains. In case of conflict, the present document, including definitions, will control. Preferred methods and materials are described below, although methods and materials similar or equivalent to those described herein can also be used in the practice or testing of the presently disclosed methods and compositions. All publications, patent applications, patents, and other references mentioned herein are incorporated by reference in their entirety.

Other features and advantages of the present disclosure will be apparent from the following description, the examples, and from the claims.

### DETAILED DESCRIPTION OF THE INVENTION

### Introduction

The purpose of this application is describe and disclose novel antibody-based agents for imaging in vivo CD8 expression. In specific embodiments, this application discloses antibody-based agents for imaging in vivo CD8 expression in mouse models of disease. This application includes the sequencing of the variable domains of anti-mouse-CD8 from hybridomas for the subsequent engineering into antibody fragments, such as, but not limited to, scFv's, diabodies, minibodies and scFv-Fc's. When the engineered antibody fragment is conjugated to a radioisotope or fluorophore and injected into a mouse, PET, SPECT, or fluorescence imaging can monitor the in vivo expression of CD8. It could be of great utility as CD8 is expressed on a subtype of T cells, among other cells, that are the subject of many research efforts, including cellular immunotherapy and tumor oncology. Imaging CD8 expression in a wide variety of preclinical CD8 therapies would be invaluable for monitoring the success/failure of various preclinical developmental immunotherapies. The ability to monitor the migration, expansion and longevity of therapeutically transferred cells using molecular imaging technologies is of critical importance for experimenting with immunomodulating therapies. Commercial applications include producing and selling ready to image antibody fragments that can monitor mouse CD8 expression for labs investigating preclinical immunotherapies around the world.

Imaging in vivo CD8 expression in mice utilizing antibody fragments is a novel concept. Antibodies have been used routinely to target CD8 T cells in vivo, but not for the purpose of imaging. These previous studies focused on the in vivo depletion of CD8 T cells or the blocking of CD8 function for immunological studies.

Other groups have genetically modified CD8 T cells with reporter genes for the monitoring of adoptively transferred cells, but this is not an antibody-based imaging approach. Imaging with the parental antibodies from the hybridoma would not be practical as they are depleting and the CD8 targeted cells would be killed/opsonized by the full length antibody. The imaging agents developed here should be biologically inert as to not deplete or activate the CD8 expressing cells in vivo.

Imaging in vivo CD8 expression utilizing antibody fragments is a novel concept. For imaging CD8 expression, the antibody engineering from a full length antibody into antibody fragments serves two purposes: first, antibody fragments do not contain the complete Fc domain and will, therefore, not deplete CD8 expressing cells in vivo, and second, the antibody fragments have optimal pharmacokinetics for imaging purposed because the lack of a full Fc domain decreases the blood half-life. A reduction of blood half-life allows for high signal-to-noise images at earlier time points.

In certain embodiments, this application is practiced by radiolabeling the desired antibody fragment with various isotopes, such as Cu-64, Zr-89, F-18 or Tc-99m, or adding a fluorescent molecule to the antibody fragment. This modified fragment is then injected into mice for PET, SPECT or fluorescent whole body imaging.

As described herein, novel anti-mouse CD8 minibodies have been constructed, produced, purified and verified for binding by flow cytometry (from hybridomas YTS 169.4.2.1, YTS 156.7.7 and 2.43). Minibodies from the hybridomas YTS 169.4.2.1 and 2.43 have been conjugated to the chelator NOTA, radiolabeled with Cu-64, and used for imaging by ImmunoPET. Also described herein are novel anti-CD8 diabodies that have been constructed, produced, purified, and verified by binding and imaging studies (e.g., ⁶⁴Cu-DOTA-2.43 cDb and ⁸⁹Zr-DFO-2.43 cDb).

Described herein are two CD8 minibodies (Mbs) for the detection of CD8 expression and the treatment of diseases associated with CD8 expression. The minibodies described herein are murine anti-CD8 minibodies and methods of making chimeric and humanized anti-CD8 minibodies for the detection and treatment of a human subject.

The two novel anti-CD8 minibodies developed here for ⁶⁴Cu immunoPET imaging of CD8 expression both retain their antigen specificity after engineering to the minibody format. Furthermore, both anti-CD8 minibodies were produced at high yields in culture and were purified using a single one-step IMAC purification. In vivo studies demonstrated that both minibodies target the spleen and lymph nodes of antigen positive mice. Furthermore, the in vivo studies have demonstrated that the anti-CD8 minibodies, that are engineered antibody fragments without the full Fc domain, do not deplete CD8 T cells in vivo, a critical feature in the development of a biologically inert imaging agent targeting immune cells. Based on this in vivo data, it is possible that the CD8 cross linking is activating in vivo due to the bivalent nature of the minibodies and diabodies.

As described herein, ImmunoPET images using the novel anti-CD8 minibodies and diabodies were acquired at relatively early times post-injection (e.g., approximately 4 hr) compared to other minibody fragments. For example, both the novel 2.43 and YTS169 minibodies have approximately 0.3% ID/g in the blood, indicating a rapid clearance of the minibodies.

This rapid clearance of the anti-CD8 minibodies and diabodies could be due to not only the reduced size of the minibodies and diabodies compared to the intact antibody, but the abundance of naturally expressed CD8 antigen throughout the body. This is termed the “antigen sink” (i.e., when there is an abundance of naturally expressed CD8 antigen, the anti-CD8 antibody will be sopped up by the naturally expressed CD8 antigen and will result in lower serum antibody levels and more rapid clearance). The antigen sink results in the rapid accumulation of the radiotracer in organs outside of the blood, but makes imaging non-antigen sink organs, e.g., a tumor, difficult. The rapid biological half-life of the novel minibodies and diabodies described herein, however, is well matched to the intermediate physical half-life of ⁶⁴Cu (12.7 hrs) immunoPET isotope compared to ¹⁸F (1.8 hrs), ¹²⁴I (100.2 hrs), and ⁸⁹Zr (78.4 hrs).

Recent studies imaging CD20 in a human CD20 expressing transgenic mouse model using ⁶⁴Cu or ⁸⁹Zr radiolabeled rituximab mimic the targeting ability of the CD8 minibodies in vivo as the abundance and location of CD8 and CD20 antigen expression are similar in vivo (Natarajan A. et al., Positron emission tomography of 64Cu-DOTA-Rituximab in a transgenic mouse model expressing human CD20 for clinical translation to image NHL. Mol Imaging Biol. October 2012; 14(5):608-616; Natarajan A. et al., Development of a Novel Long-Lived ImmunoPET Tracer for Monitoring Lymphoma Therapy in a Humanized Transgenic Mouse Model. Bioconjug Chem. Jun. 11 2012). However in the rituximab studies, the radiolabeled rituximab was biologically active and was therefore not ideal for imaging studies.

Engineering to other antibody formats can also decrease the Fc-dependent biological activity of the imaging radiopharmaceutical (Olafsen T. et al., ImmunoPET imaging of B-cell lymphoma using 124I-anti-CD20 scFv dimers (diabodies). Protein Eng Des Sel. April 2010; 23(4):243-249; Olafsen T. et al., Recombinant anti-CD20 antibody fragments for small-animal PET imaging of B-cell lymphomas. J Nucl Med. September 2009; 50(9):1500-1508). However, it is important to note that the radiolabeled intact rituximab in these studies by Natarajan et al. showed rapid clearance from the blood pool. (Natarajan A. et al., Positron emission tomography of ⁶⁴Cu-DOTA-Rituximab in a transgenic mouse model expressing human CD20 for clinical translation to image NHL. Mol Imaging Biol. October 2012; 14(5):608-616; Natarajan A. et al., Development of a Novel Long-Lived ImmunoPET Tracer for Monitoring Lymphoma Therapy in a Humanized Transgenic Mouse Model. Bioconjug Chem. Jun. 11 2012). While this rapid clearance may be due to the abundance of naturally expressed CD20 antigen, it could also be due to the depletion of CD20 expression cells over the 48 hour study (Natarajan A. et al., Positron emission tomography of 64Cu-DOTA-Rituximab in a transgenic mouse model expressing human CD20 for clinical translation to image NHL. Mol Imaging Biol. October 2012; 14(5):608-616; Natarajan A. et al., Development of a Novel Long-Lived ImmunoPET Tracer for Monitoring Lymphoma Therapy in a Humanized Transgenic Mouse Model. Bioconjug Chem. Jun. 11 2012).

In the context of tumor targeting, the CD20 antigen sink has been overcome by blocking the antigen sink with cold antibody either during (bolus injection) or before (predosing/blocking injection) the injection of the radiotracer. Blocking studies were performed in the human CD20 transgenic model that resulted in an increased radiotracer blood half-life that could greatly influence the ability to target CD20+ B cell lymphomas in vivo. In fact, the FDA-approved radioimmunotherapeutic Zevalin, a ⁹⁰Y radiolabeled anti-CD20 antibody, which requires a predose of cold rituximab for therapy in order to block accumulation of the ⁹⁰Y radioimmunotherapeutic in the spleen and increase targeting of lymphoma cells.

This technique of bolus or predosing injections has proven important for targets other than immunological cell surface molecules where antigen sinks exist. Bolus injections have been used, for example, in a ⁸⁹Zr radiolabeled trastuzumab immunoPET study that required high doses for reliable targeting in patients due to high levels of shed extracellular domain of HER2 in the plasma (Dijkers E. C. et al., Biodistribution of ⁸⁹Zr-trastuzumab and PET imaging of HER2-positive lesions in patients with metastatic breast cancer. Clin Pharmacol Ther. May 2010; 87(5):586-592). Also, both ¹¹¹In radiolabeled anti-EFGR and anti-VEGFR antibodies demonstrated high lung and/or liver uptake that could be reduced while enhancing tumor uptake when higher protein doses were injected (Bumbaca D. et al., Maximizing tumour exposure to anti-neuropilin-1 antibody requires saturation of non-tumour tissue antigenic sinks in mice. Br J Pharmacol. May 2012; 166(1):368-377; Divgi C. R. et al., Phase I and imaging trial of indium 111-labeled anti-epidermal growth factor receptor monoclonal antibody 225 in patients with squamous cell lung carcinoma. J Natl Cancer Inst. Jan. 16 1991; 83(2):97-104).

However, the concept of blocking the antigen sink has repercussions in the field of both therapeutic antibody-drug conjugates and imaging. For example, predosing injections can be used to block the antigen sink of tomoregulin, or TENB2, a transmembrane protein overexpressed in prostate tumors, in order to increase the therapeutic index of the monomethyl auristatin E conjugated anti-TENB2 antibody (Boswell C. A. et al., Differential effects of predosing on tumor and tissue uptake of an ¹¹¹In-labeled anti-TENB2 antibody-drug conjugate. J Nucl Med. September 2012; 53(9):1454-1461). This study also highlights potential of displacing the fine balance between efficient blocking of the antigen sink versus displacing the tumor uptake. Accordingly, the ability of the novel anti-CD8 minibody and diabody fragments described herein to image either CD8⁺ lymphomas or tumor infiltrating CD8⁺ T cells, for example, may rely on efficiently blocking the antigen sink for consistent targeting in certain embodiments.

The rapid clearance of the anti-CD8 minibodies and diabodies could also be due to the presence of multimers causing increased liver uptake at early time points. For example, at 4 hr post-injection, the CD8 minibodies have ˜60-70% ID/g in the liver, compared to other ⁶⁴Cu radiolabeled minibodies that range from 15-32.4% ID/g at 4-5 hr post-injection (Wu A. M. et al., High-resolution microPET imaging of carcinoembryonic antigen-positive xenografts by using a copper-64-labeled engineered antibody fragment. Proc Natl Acad Sci USA. Jul. 18 2000; 97(15):8495-8500; Olafsen T. et al., Optimizing radiolabeled engineered anti-p185HER2 antibody fragments for in vivo imaging. Cancer Res. Jul. 1 2005; 65(13):5907-5916). Non-specific liver uptake and retention occurs when using ⁶⁴Cu due to the transchelation of copper to enzymes in the liver (Rogers B. E. et al., Comparison of four bifunctional chelates for radiolabeling monoclonal antibodies with copper radioisotopes: biodistribution and metabolism. Bioconjug Chem. July-August 1996; 7(4):511-522). However, a minibody dimer (˜160 kDa) is similar in size as an intact antibody but lack the Fc domain, thereby decreasing liver retention.

As described herein, B/6 and C3H mice injected with the ⁶⁴Cu-NOTA-YTS169 minibody showed decreased uptake in lymph nodes, spleen, and blood when compared to the ⁶⁴Cu-NOTA-2.43 minibody. This could be attributed to the higher amount of multimer in the YTS169 minibody than the 2.43 Minibody, as shown by SEC, causing increased hepatic clearance, resulting in a decreased blood half-life and lower ability to target lymph nodes. Furthermore, it is important to note that the minibody dimers of both YTS169 and 2.43 retain their ability to bind sCD8αβ as shown by size exclusion and SPR. Potential aggregation and dimerization due to inter-V_{H}-V_{L} binding can be reduced, for example, by engineering two cysteines that stabilize the interaction between V_{H} and V_{L}, among other methods (Glockshuber R. et al., A comparison of strategies to stabilize immunoglobulin Fv-fragments. Biochemistry. Feb. 13 1990; 29(6):1362-1367; Worn A. et al., Stability engineering of antibody single-chain Fv fragments. J Mol Biol. Feb. 2 2001; 305(5):989-1010).

An unexpected discrepancy of the ⁶⁴Cu-NOTA-YTS169 minibody that binds both Lyt2.1 and Lyt2.2 is the difference between the biodistributions in B/6 and C3H mice. In this study, however, the C3H mice were 20 weeks old and the B/6 mice were 8 weeks old with average spleen and liver weights of either approximately 103 mg and 880 mg or approximately 52 mg and 650 mg, respectively. Therefore, the weights of the organs greatly effect the % ID/g and the actual % ID/organ four hours post-injection is similar in the spleen and liver. This highlights an important fact that % ID/g values are not consistent for every experiment but they are very reproducible among groups of mice of the same age and weight.

The development of the novel anti-CD8 immunoPET radiotracers and minibodies and diabodies disclosed herein are beneficial for studying a host of preclinical disease models, including, but not limited to, lymphoma detection and tumor T cell infiltration, with specific importance relating to in vivo, non-invasive use. Furthermore, preclinical immunotherapy models enhancing cytotixic T cell functions can be monitored by immunoPET as disclosed herein the development and translation of anti-human CD8 antibody fragments for immunoPET imaging in the clinic can be made.

### CD8

CD8 is a cell surface glycoprotein expressed mainly on a subset of T cells, known as cytotoxic T cells, and a subset of dendritic cells. It is a coreceptor for MHC class I molecules to enhance antigen presentation to T cell receptors. CD8 functions as a coreceptor for TCR recognition of peptide antigen complexed with MHC Class I molecule (pMHCI).

T cells act as effectors of the immune response. One of the most striking ways in which they do so is by targeting cells displaying foreign antigen. The subset of T cells that mediate this lytic function are designated as cytotoxic T lymphocytes (CTL). The highly specific nature of the CTL response is apparent in cell-mediated responses to viral infections and to allografts. This sub-population of lymphocytes is characterized by expression of the cell surface marker CD8. The CD8 protein has been shown to play a major role in both activation of mature T-cells and the thymic differentiation process that leads to expression of CD8. Classically, CD8 has been viewed as an accessory molecule involved in ligation of class I major histocompatibility complex (MHC) bearing antigen on an antigen presenting cell (APC). In recent years, accumulating evidence suggests that this model for the role of CD8 in T cell activation is not complete. It is now believed that CD8 plays a major role in signal pathways leading to T cell proliferation (Miceli and Parnes, Adv. In Immuno. 53:59-72 (1993)).

CD8 has been shown to physically associate with the T cell receptor complex (TCR), as demonstrated by co-immunoprecipitation and by co-capping experiments (Gallagher et al., PNAS 86:10044-10048 (1989)). TCR signalling and TCR mediated lymphokine production are markedly enhanced with CD8-TCR aggregation. Characterization of the CD8 structure by a panel of monoclonal antibodies directed against CD8 showed that MHC class I binding and TCR interaction are associated with distinct regions of the CD8 molecule (Eichmann et al., J. of Immuno. 147:2075-2081 (1991). In addition, CD8 and the TCR recognize the same class I molecule (Connoly et al., PNAS 87:2137-2141 (1990)).

The human CD8 molecule is expressed either as an αα homodimer or as an αβ heterodimer. Individual human peripheral T-cells can express varying amounts of CD8 αα and αβ complexes, and their relative ratios appear to be differentially regulated upon T-cell activation. The biological activity of CD8 has primarily been attributed to the α chain, which enhances or reconstitutes T-cell responses in the homodimeric form. In contrast, until recently, no role had been ascribed to the β chain.

Mice that are chimeric for the homozygous disruption of the CD8 β gene developed normally to the CD4+ CD8+ stage, but did not efficiently differentiate further, which results in a low number of peripheral CD8+ T-cells. The fact that the number of peripheral CD8+ T-cells was restored upon transfer of exogenous CD8 β gene indicates that CD8 β is necessary for the maturation of CD8+ T-cells. It has also been shown that CD8 αβ transfectants produce more IL-2 than CD8 αα transfectants in response to specific stimuli (Wheeler et al., Nature 357:247-249 (1992)). T-cell activation results in the physical modification of the mouse CD8 β chain shown by the reversible alteration in its sialic acid content (Casabo et al., J. of Immuno. 152:397-404 (1994)). This modification may influence the physical structure of the CD8 complex and in turn the interaction with TCR and MHC class I. The gene encoding the CD8 molecule has been cloned for several species (human, rat, mouse) (Sukhame et al., Cell 40:591-597 (1985); Nakauchi et al, PNAS 82:5126-5130 (1985)).

The murine CD8 molecule is expressed as a heterodimeric structure consisting of two disulfide linked subunits; Lyt-2, which has a molecular weight of about 38 kDa and Lyt-3, which has a molecular weight of 30 kDa (Ledbetter et al., J. of Exp. Med. 153:1503-1516 (1981)). The .alpha. chain gene can also undergo an alternative mode of mRNA splicing resulting in expression of the α form which is distinguishable from a by its shorter cytoplasmic tail (Zamoyska et al., Nature 342:278 (1989); Giblin et al., PNAS 86:998-1002 (1989)).

Sequence analysis of CD8 indicates that it is a member of the immunoglobulin (Ig) superfamily. Members of the Ig-superfamily exhibit highly conserved hydrophobic cores. The CD8 molecule consists of an unique amino-terminal Ig-variable domain, an extracellular spacer which carries the structural features of Ig hinge-line region, a transmembrane domain and an intracellular cytoplasmic tail. The crystal structure of the extracellular Ig-like portion of the homodimeric human CD8 α has been recently solved (Leahy et al., Cell 68:1145-1162 (1992)). The amino-terminal domain of the CD8 chain was shown to closely resemble an Ig-variable region. The regions that are analogous to antigen-binding domains on an immunoglobulin protein are referred to as the complementarity determining regions (CDRs). Recent mutagenesis studies of the different domains of CD8 has indicated that CDR1 and CDR2 like domains are involved in MHC class I interactions (Sanders et al., J. of Exp. Med. 174:371-379 (1991)).

Replacement of the human CD8 α CDR2-like loop by the homologous mouse sequences results in the loss of interaction of monoclonal antibodies that are capable of inhibiting CD2-mediated Ca⁺² increases (Franco et al, Cellular Immuno. 157:341-352 (1994)). This suggests that the CDR2-like region of CD8 α-chain may be involved in regulating T-cell activation.

These data indicate that the role of CD8 in MHC class I interaction is not incidental, but required for efficient stimulation of the T cell. The CD8 molecule plays a role very similar, yet distinct, to that of CD4 in class II MHC-restricted activation. Thus, CD8 must be involved in the regulation of a complex system of modulation of signalling involving many closely related molecules.

Accordingly, there is a need for pharmaceutical compositions which can effectively inhibit the immune responses mediated by CD8 activity. There is a need for a method of inhibiting CD8 mediated T cell activation. There is a need for pharmaceutical compositions which can effectively inhibit CD8 mediated diseases. There is a need for pharmaceutical compositions for non-invasive in vivo imaging.

The CD8 co-receptor is predominantly expressed on the surface of cytotoxic T cells. CD8 can also be found, for example, on natural killer cells, cortical thymocytes, and dendritic cells. It is expressed in T cell lymphoblastic lymphoma and hypo-pigmented mycosis fungicides, but is frequently lost in other T-cell neoplasms.

CD8+ T cells may function in more than one way. The best known function is the killing or lysis of target cells bearing peptide antigen in the context of an MHC class I molecule. Hence these cells are often termed cytotoxic T lymphocytes (CTL). However, another function, perhaps of greater protective relevance in malaria infections is the ability of CD8+ T cells to secrete interferon gamma (IFN-γ).

CTLs are the arm of adaptive immunity responsible for the recognition and elimination of infected cells, tumor cells, and allogeneic cells. Once primed, CTL can recognize their target antigen on a wide variety of cells and accomplish their function by lysing the target cell and/or secreting cytokines like TNF-alpha, or IFN-gamma. Presentation of antigen to CD8+ CTL (cytotoxic T lymphocytes) occurs in the context of MHC class I molecules (MHC-I), while presentation of antigen to CD4+ HTL (helper T lymphocytes) occurs in the context of MHC class II molecules. Accordingly, the induction of a CD8 mediated immune response to a cellular antigen (derived from a tumor cell or an infected cell) can, for example, comprise dendritic cells that acquire antigens derived from tumor or infected cells; thereafter, interaction of DC-antigen with CD4 cells can, for example, enable the DC to activate the CD8 cells.

As described above, mice have two alleles for CD8a, Lyt2.1 and Lyt2.2, that are restricted to certain mouse strains. Lyt2.1, for example, is expressed in the mouse strains CBA, AKR, C3H and DBA while Lyt2.2 is expressed in the mouse strains Balb/c and C57BL/6 (B/6). The difference between Lyt2.1 and Lyt2.2 is a methionine (Lyt2.2) to valine (Lyt2.1) substitution at residue 78 of the mature CD8a.

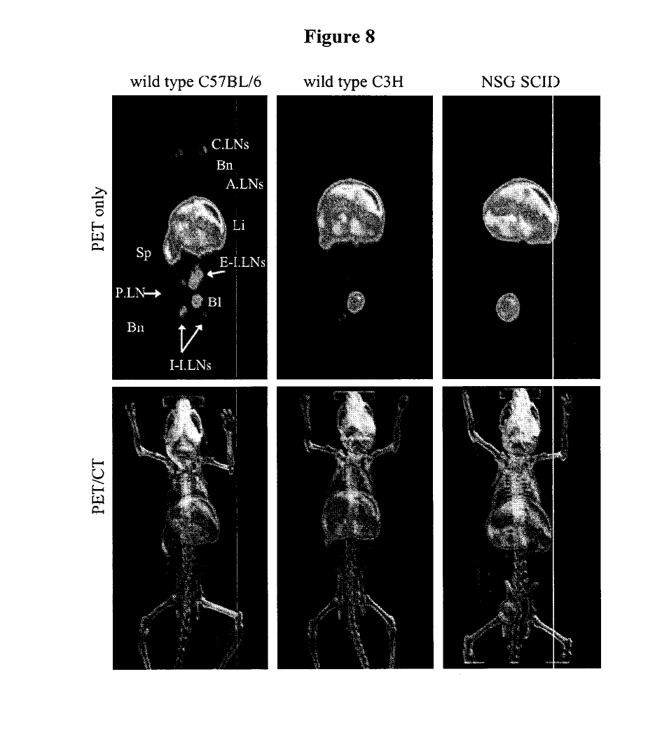

Disclosed herein, the parental antibodies from the hybridomas YTS 169.4.2.1 (YTS169) and 2.43 were engineered into minibody and diabody fragments. Both the YTS169 and 2.43 antibodies bind mCD8+. However, they differ in that the YTS169 antibody binds both Lyt2.1 and Lyt2.2 while the 2.43 antibody binds an epitope that is Lyt2.2 specific. These novel minibodies retained their antigen specificity as shown by flow cytometry and ⁶⁴Cu-immunoPET imaging. Most importantly, both the 2.43 and YTS169 minibodies and diabodies described herein produce high contrast ImmunoPET images of CD8⁺ lymphoid organs at only four hours post-injection (FIGS. 8-11 and 16-17).

### Antibodies

Antibodies that find use in the present invention can take on a number of formats such as traditional minibody antibodies as well as their derivatives, fragments and mimetics. In certain embodiments of this invention, the anti-CD8 minibodies are mouse anti-CD8 minibodies YTS169 and 2.43. In other embodiments, the anti-CD8 minibodies are chimeric or humanized forms of the YTS169 and 2.43 minibodies that can be used for in vivo imaging and treatment in humans. These antibodies and their use are described herein.

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Thus, “isotype” as used herein is meant any of the subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. It should be understood that therapeutic antibodies can also comprise hybrids of isotypes and/or subclasses.

The amino-terminal portion of each chain includes a variable region of about 100 to 110 or more amino acids primarily responsible for antigen recognition. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. “Variable” refers to the fact that certain segments of the variable region differ extensively in sequence among antibodies. Variability within the variable region is not evenly distributed. Instead, the V regions consist of relatively invariant stretches called framework regions (FRs) of 15-30 amino acids separated by shorter regions of extreme variability called “hypervariable regions” that are each 9-15 amino acids long or longer.

Each VH and VL is composed of three hypervariable regions (“complementary determining regions,” “CDRs”) and four FRs, arranged from amino-terminus to carboxy-terminus in the following order: FR1-CDR1-FR2-CDR2-FR3-CDR3-FR4.

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5^{th} Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

Throughout the present specification, the Kabat numbering system is generally used when referring to a residue in the variable domain (approximately, residues 1-107 of the light chain variable region and residues 1-113 of the heavy chain variable region) (e.g, Kabat et al., supra (1991)).

The CDRs contribute to the formation of the antigen-binding, or more specifically, epitope binding site of antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope. For example, as described herein the antibodies bind to CD8.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

In some embodiments, the YTS169 minibody binds both Lyt2.1 and Lyt2.2 epitopes while the 2.43 minibody binds an epitope that is Lyt2.2 specific.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.”

The carboxy-terminal portion of each chain defines a constant region primarily responsible for effector function. Kabat et al. collected numerous primary sequences of the variable regions of heavy chains and light chains. Based on the degree of conservation of the sequences, they classified individual primary sequences into the CDR and the framework and made a list thereof (see SEQUENCES OF IMMUNOLOGICAL INTEREST, 5^{th} edition, NIH publication, No. 91-3242, E. A. Kabat et al., entirely incorporated by reference).

In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the heavy chain is the hinge region. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230.

Of interest in the present invention are the Fc regions. By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

In some embodiments, the antibodies are minibodies. Alternatively, the antibodies can be a variety of structures, including, but not limited to, antibody fragments, monoclonal antibodies, bispecific antibodies, full length, domain antibodies, synthetic antibodies (sometimes referred to herein as “antibody mimetics”), chimeric antibodies, humanized antibodies, antibody fusions (sometimes referred to as “antibody conjugates”), and fragments of each, respectively.

In one embodiment, the antibody is an antibody fragment. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546, entirely incorporated by reference) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883, entirely incorporated by reference), (viii) bispecific single chain Fv (WO 03/11161, hereby incorporated by reference) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448, all entirely incorporated by reference).

In some embodiments, the antibody can be a mixture from different species, e.g. a chimeric antibody and/or a humanized antibody. That is, in the present invention, the CDR sets can be used with framework and constant regions other than those specifically described by sequence herein.

In general, both “chimeric antibodies” and “humanized antibodies” refer to antibodies that combine regions from more than one species. For example, “chimeric antibodies” traditionally comprise variable region(s) from a mouse (or another animal such as a rat, a rabbit, a goat, or a chicken) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system. Roque et al., 2004, Biotechnol. Prog. 20:639-654, entirely incorporated by reference.

A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

In one embodiment, the antibodies of the invention can be multispecific antibodies, and notably bispecific antibodies. These are antibodies that bind to two (or more) different antigens, or different epitopes on the same antigen.

In a specific embodiment, the antibody is a minibody. Minibodies are minimized antibody-like proteins comprising a scFv joined to a CH3 domain. Hu et al., 1996, Cancer Res. 56:3055-3061, entirely incorporated by reference. In some cases, the scFv can be joined to the Fc region, and may include some or the entire hinge region.

In a specific embodiment, the antibody is a diabody.

The antibodies of the present invention are generally isolated or recombinant. An “isolated antibody,” refers to an antibody which is substantially free of other antibodies having different antigenic specificities. For instance, an isolated antibody that specifically binds to CD8 is substantially free of antibodies that specifically bind antigens other than CD8.

An isolated antibody that specifically binds to an epitope, isoform or variant of human CD8 or murine CD8 may, however, have cross-reactivity to other related antigens, for instance from other species, such as CD8 species homologs. Moreover, an isolated antibody may be substantially free of other cellular material and/or chemicals.

Isolated monoclonal antibodies, having different specificities, can be combined in a well defined composition. Thus, for example all possible combinations of the YTS169 minibody and the 2.43 minibody can be combined in a single formulation, if desired. In another embodiment, all possible combinations of the YTS169 diabody and the 2.43 diabody can be combined in a single formulation, if desired. In another embodiment, all possible combinations of the YTS169 and 2.43 minibodies or diabodies can be combined in a single formulation, if desired.

In certain embodiments the anti-mCD8 YTS169 cDb nucleic acid sequence (SEQ ID NO:1):

GAAGTGAAGCTGCAGGAAAGCGGCGGAGGCCTGGTGCAGCCCGGCAGAAG
CCTGAAGCTGAGCTGTGCCGCCAGCGGCTTCAACTTCAACGACTACTGGA
TGGGCTGGGTCCGACAGGCTCCTGGCAAGGGCCTGGAATGGATCGGCGAG
ATCAACAAGGACAGCAGCACCATCAACTACACCCCCAGCCTGAAGGACAA
GTTCACCATCAGCAGAGACAACGCCCAGAACACCCTGTACCTGCAGATGA
GCAAGCTGGGCAGCGAGGACACCGCCATCTACTACTGCGCCAGAGCCAGA
GGCATGATGGTGCTGATCATCCCCCACTACTTCGACTACTGGGGCCAGGG
CGTGATGGTCACCGTGTCCAGCGGCGGCGGAGGAAGCGACATCGTGCTGA
CCCAGAGCCCCGCTATGGCCATGAGCCCTGGCGAGAGAATCACAATCAGC
TGCAGAGCCAGCGAGAGCGTGTCCACCAGAATGCACTGGTATCAGCAGAA
GCCCGGCCAGCAGCCCAAGCTGCTGATCTACGGCGCCAGCAACCTGGAAT
CCGGCGTGCCAGCCAGATTCAGCGGCAGCGGCTCCGGCACCGACTTCACC
CTGACCATCGACCCCGTGGAAGCCAACGACACCGCCACCTATTTCTGCCA
GCAGTCTTGGTACGACCCCTGGACCTTCGGTGGAGGCACCAAGCTGGAAC
TGAAGGCGGCCGCAGGCTGCGGAGGCCACCACCATCATCACCATTGATAG

In certain embodiments the anti-mCD8 YTS 169 cDb amino acid sequence (SEQ ID NO:2):

EVKLQESGGGLVQPGRSLKLSCAASGFNFNDYWMGWVRQAPGKGLEWIGE
INKDSSTINYTPSLKDKFTISRDNAQNTLYLQMSKLGSEDTAIYYCARAR
GMMVLIIPHYFDYWGQGVMVTVSSGGGGSDIVLTQSPAMAMSPGERITIS
CRASESVSTRMHWYQQKPGQQPKLLIYGASNLESGVPARFSGSGSGTDFT
LTIDPVEANDTATYFCQQSWYDPWTFGGGTKLELKAAAGCGGHHHHHH**

In certain embodiments the anti-mCD8 2.43 cDb nucleic acid sequence (SEQ ID NO:3):

GAAGTGCAGCTGGTGGAAAGCGGCGGAGGCCTGGTGCAGCCCGGCAGAAG
CCTGAAGCTGAGCTGTGCCGCCAGCGGCTTCACCTTCAGCAACTACTACA
TGGCTTGGGTGCGCCAGGCCCCCACCAAGGGACTGGAATGGGTGGCCTAC
ATCAACACCGGCGGAGGCACCACCTACTACAGAGACAGCGTGAAGGGCAG
ATTCACCATCAGCAGGGACGACGCCAAGAGCACCCTGTACCTGCAGATGG
ACAGCCTGAGAAGCGAGGACACCGCTACCTACTACTGCACCACCGCCATC
GGCTACTACTTCGACTACTGGGGCCAGGGCGTGATGGTGACAGTGTCCAG
CGGTGGCGGAGGAAGCGACATCCAGCTGACACAGAGCCCCGCCAGCCTGA
GCGCCTCTCTGGGCGAGACAGTGTCTATCGAGTGCCTGGCCAGCGAGGAC
ATCTACAGCTACCTGGCCTGGTATCAGCAGAAGCCCGGCAAGAGCCCCCA
GGTGCTGATCTACGCCGCCAACAGACTGCAGGACGGCGTGCCCAGCAGAT
TCAGCGGCTCTGGCAGCGGCACCCAGTACAGCCTGAAGATCAGCGGCATG
CAGCCCGAGGACGAGGGCGACTACTTCTGTCTGCAGGGCAGCAAGTTCCC
CTACACCTTCGGCGCTGGCACCAAGCTGGAACTGAAGGCGGCCGCAGGCT
GCGGAGGCCACCACCATCATCACCATTGATAG

In certain embodiments the anti-mCD8 2.43 cDb amino acid sequence (SEQ ID NO:4):

EVQLVESGGGLVQPGRSLKLSCAASGFTFSNYYMAWVRQAPTKGLEWVAY
INTGGGTTYYRDSVKGRFTISRDDAKSTLYLQMDSLRSEDTATYYCTTAI
GYYFDYWGQGVMVTVSSGGGGSDIQLTQSPASLSASLGETVSIECLASED
IYSYLAWYQQKPGKSPQVLIYAANRLQDGVPSRFSGSGSGTQYSLKISGM
QPEDEGDYFCLQGSKFPYTFGAGTKLELKAAAGCGGHHHHHH**

In certain embodiments the anti-mCD8 YTS 156 cDb nucleic acid sequence (SEQ ID NO:5):

GAAGTGAAGCTGCAGGAAAGCGGCCCCAGCCTGGTGCAGCCTAGCCAGAC
CCTGAGCCTGACCTGCAGCGTGTCCGGCTTCAGCCTGATCAGCGACAGCG
TGCACTGGGTCCGACAGCCTCCCGGCAAGGGCCTGGAATGGATGGGCGGC
ATCTGGGCCGACGGCTCCACCGACTACAACAGCGCCCTGAAGTCCAGACT
GAGCATCAGCAGAGACACCAGCAAGAGCCAGGGCTTCCTGAAGATGAACA
GCCTGCAGACCGACGACACCGCCATCTATTTCTGCACCAGCAACCGCGAG
AGCTACTACTTCGACTACTGGGGCCAGGGCGTGATGGTCACCGTGTCCAG
CGGCGGCGGAGGCTCTGACATCCAGATGACCCAGAGCCCTGCCAGCCTGA
GCGCCAGCCTGGGCGACAAAGTGACCATCACCTGTCAGGCCAGCCAGAAC
ATCGACAAGTATATCGCCTGGTATCAGCAGAAGCCTGGCAAGGCCCCCAG
ACAGCTGATCCACTACACCAGCACACTGGTGTCCGGCACCCCCAGCAGAT
TCAGCGGCAGCGGTTCCGGCAGAGACTACAGCTTCAGCATCAGCTCCGTG
GAAAGCGAGGATATCGCCAGCTACTACTGCCTGCAGTACGACACCCTGTA
CACCTTCGGCGCTGGCACCAAGCTGGAACTGAAGGCGGCCGCAGGCTGCG
GAGGCCACCACCATCATCACCATTGATAG

In certain embodiments the anti-mCD8 YTS 156 cDb amino acid sequence (SEQ ID NO:6):

EVKLQESGPSLVQPSQTLSLTCSVSGFSLISDSVHWVRQPPGKGLEWMGG
IWADGSTDYNSALKSRLSISRDTSKSQGFLKMNSLQTDDTAIYFCTSNRE
SYYFDYWGQGVMVTVSSGGGGSDIQMTQSPASLSASLGDKVTITCQASQN
IDKYIAWYQQKPGKAPRQLIHYTSTLVSGTPSRFSGSGSGRDYSFSISSV
ESEDIASYYCLQYDTLYTFGAGTKLELKAAAGCGHHHHHH**

In certain embodiments the V_{H} of the anti-mCD8 YTS 169 antibody_amino acid sequence (SEQ ID NO:7):

EVKLQESGGGLVQPGRSLKLSCAASGFNFNDYWMGWVRQAPGKGLEWIGE
INKDSSTINYTPSLKDKFTISRDNAQNTLYLQMSKLGSEDTAIYYCARAR
GMMVLIIPHYFDYWGQGVMVTVSS

In certain embodiments the CDR1 of the V_{H} of the anti-mCD8 YTS 169 antibody amino acid sequence (SEQ ID NO:8):

GFNFNDY

In certain embodiments the CDR2 of the V_{H} of the anti-mCD8 YTS 169 antibody amino acid sequence (SEQ ID NO:9):

EINKDSSTINYTPSLKD

In certain embodiments the CDR2 of the V_{H} of the anti-mCD8 YTS 169 antibody amino acid sequence (SEQ ID NO:10):

ARGMMVLIIPHYFDY

In certain embodiments the V_{L} of the anti-mCD8 YTS 169 antibody amino acid sequence (SEQ ID NO:11):

DIVLTQSPAMAMSPGERITISCRASESVSTRMHWYQQKPGQQPKLLIYGA
SNLESGVPARFSGSGSGTDFTLTIDPVEANDTATYFCQQSWYDPWTFGGG
TKLELK

In certain embodiments the CDR1 of the V_{L} of the anti-mCD8 YTS 169 antibody amino acid sequence (SEQ ID NO:12):

RASESVSTRMH

In certain embodiments the CDR2 of the V_{L} of the anti-mCD8 YTS 169 antibody amino acid sequence (SEQ ID NO:13):

GASNLES

In certain embodiments the CDR3 of the V_{L} of the anti-mCD8 YTS 169 antibody amino acid sequence (SEQ ID NO:14):

QQSWYDPWT

In certain embodiments the V_{H} of the anti-mCD8 2.43 antibody amino acid sequence (SEQ ID NO:15):

EVQLVESGGGLVQPGRSLKLSCAASGFTFSNYYMAWVRQAPTKGLEWV
AYINTGGGTTYYRDSVKGRFTISRDDAKSTLYLQMDSLRSEDTATYYC
TTAIGYYFDYWGQGVMVTVSS

In certain embodiments the CDR1 of the V_{H} of the anti-mCD8 2.43 antibody amino acid sequence (SEQ ID NO:16):

GFTFSNYYMA

In certain embodiments the CDR2 of the V_{H} of the anti-mCD8 2.43 antibody amino acid sequence (SEQ ID NO:17):

YINTGGGTTYYRDSV

In certain embodiments the CDR3 of the V_{H} of the anti-mCD8 2.43 antibody amino acid sequence (SEQ ID NO:18):

AIGYYFDY

In certain embodiments the V_{L} of the anti-mCD8 2.43 antibody amino acid sequence (SEQ ID NO:19):

DIQLTQSPASLSASLGETVSIECLASEDIYSYLAWYQQKPGKSPQVLI
YAANRLQDGVPSRFSGSGSGTQYSLKISGMQPEDEGDYFCLQGSKFPY
TFGAGTKLELK

In certain embodiments the CDR1 of the V_{L} of the anti-mCD8 2.43 antibody amino acid sequence (SEQ ID NO:20):

LASEDIYSYLA

In certain embodiments the CDR2 of the V_{L} of the anti-mCD8 2.43 antibody amino acid sequence (SEQ ID NO:21):

AANRLQD

In certain embodiments the CDR3 of the V_{L} of the anti-mCD8 2.43 antibody amino acid sequence (SEQ ID NO:22):

LQGSKFPYT

In certain embodiments the V_{H} of the anti-mCD8 YTS 156 antibody amino acid sequence (SEQ ID NO:23):

EVKLQESGPSLVQPSQTLSLTCSVSGFSLISDSVHWVRQPPGKGLEWM
GGIWADGSTDYNSALKSRLSISRDTSKSQGFLKMNSLQTDDTAIYFCT
SNRESYYFDYWGQGVMVTVSS

In certain embodiments the CDR1 of the V_{H} of the anti-mCD8 YTS 156 antibody amino acid sequence (SEQ ID NO:24):

GFSLISDSVH

In certain embodiments the CDR1 of the V_{H} of the anti-mCD8 YTS 156 antibody amino acid sequence (SEQ ID NO:25):

GSTDYNSALKSRLSIS

In certain embodiments the CDR1 of the V_{H} of the anti-mCD8 YTS 156 antibody amino acid sequence (SEQ ID NO:26):

NRESYYFDY

In certain embodiments the V_{L} of the anti-mCD8 YTS 156 antibody amino acid sequence (SEQ ID NO:27):

DIQMTQSPASLSASLGDKVTITCQASQNIDKYIAWYQQKPGKAPRQLI
HYTSTLVSGTPSRFSGSGSGRDYSFSISSVESEDIASYYCLQYDTLYT
FGAGTKLELK

In certain embodiments the CDR1 of the V_{L} of the anti-mCD8 YTS 156 antibody amino acid sequence (SEQ ID NO:28):

QASQNIDKYIA

In certain embodiments the CDR1 of the V_{L} of the anti-mCD8 YTS 156 antibody amino acid sequence (SEQ ID NO:29):

TSTLVS

In certain embodiments the CDR1 of the V_{L} of the anti-mCD8 YTS 156 antibody amino acid sequence (SEQ ID NO:30):

LQYDTLYT

In some embodiments these antibodies lack the polyhistine tag. In other embodiments, the antibodies are substantially identical in sequence to a minibody described herein with or without the polyhistidine tag. In still other embodiments, the antibodies are substantially identical in sequence to anti-CD8 minibody and diabody sequences disclosed herein. These identities can be 65%, 70%, 75%, 80%, 85%, 90%, and preferably 91%, 92%, 93%, 94%, 95%, 96%, 97%, 98% or 99% or greater amino acid sequence identity. In some further embodiments of any of the above, the antibodies are identical in sequence to anti-CD8 minibody sequences disclosed herein.

The anti-CD8 antibodies of the present invention specifically bind CD8 (e.g., human and murine CD8 sequences that are known of one of skill in the art).

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

The present invention further provides variant antibodies. That is, there are a number of modifications that can be made to the antibodies of the invention, including, but not limited to, amino acid modifications in the CDRs (affinity maturation), amino acid modifications in the Fc region, glycosylation variants, covalent modifications of other types, etc.

By “variant” herein is meant a polypeptide sequence that differs from that of a parent polypeptide by virtue of at least one amino acid modification. Amino acid modifications can include substitutions, insertions and deletions, with the former being preferred in many cases.

In general, variants can include any number of modifications, as long as the function of the protein is still present, as described herein. That is, in the case of amino acid variants generated with the 2.43 or YTS169 minibodies and diabodies, for example, the antibody should still specifically bind to both human and/or murine CD8. Similarly, if amino acid variants are generated with the Fc region, for example, the variant antibodies should maintain the required receptor binding functions for the particular application or indication of the antibody.

However, in general, from 1, 2, 3, 4, 5, 6, 7, 8, 9 or 10 amino acid substitutions are generally utilized as often the goal is to alter function with a minimal number of modifications. In some cases, there are from 1 to 5 modifications, with from 1-2, 1-3 and 1-4 also finding use in many embodiments.

It should be noted that the number of amino acid modifications may be within functional domains: for example, it may be desirable to have from 1-5 modifications in the Fc region of wild-type or engineered proteins, as well as from 1 to 5 modifications in the Fv region, for example. A variant polypeptide sequence will preferably possess at least about 80%, 85%, 90%, 95% or up to 98 or 99% identity to the parent sequences (e.g. the 2.43 or YTS169 minibody sequences). It should be noted that depending on the size of the sequence, the percent identity will depend on the number of amino acids.

By “amino acid substitution” or “substitution” herein is meant the replacement of an amino acid at a particular position in a parent polypeptide sequence with another amino acid. By “amino acid insertion” or “insertion” as used herein is meant the addition of an amino acid at a particular position in a parent polypeptide sequence. By “amino acid deletion” or “deletion” as used herein is meant the removal of an amino acid at a particular position in a parent polypeptide sequence.

By “parent polypeptide”, “parent protein”, “precursor polypeptide”, or “precursor protein” as used herein is meant an unmodified polypeptide that is subsequently modified to generate a variant. In general, the parent polypeptides herein are 2.43 or YTS169. Parent polypeptide may refer to the polypeptide itself, compositions that comprise the parent polypeptide, or the amino acid sequence that encodes it. Accordingly, by “parent Fc polypeptide” as used herein is meant an Fc polypeptide that is modified to generate a variant, and by “parent antibody” as used herein is meant an antibody that is modified to generate a variant antibody.

By “wild type” or “WT” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been intentionally modified.

By “variant Fc region” herein is meant an Fc sequence that differs from that of a wild-type Fc sequence by virtue of at least one amino acid modification. Fc variant may refer to the Fc polypeptide itself, compositions comprising the Fc variant polypeptide, or the amino acid sequence.

In some embodiments, one or more amino acid modifications are made in one or more of the CDRs of the antibody (2.43 or YTS169). In general, only 1 or 2 or 3 amino acids are substituted in any single CDR, and generally no more than from 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

In some cases, amino acid modifications in the CDRs are referred to as “affinity maturation.” An “affinity matured” antibody is one having one or more alteration(s) in one or more CDRs which results in an improvement in the affinity of the antibody for antigen, compared to a parent antibody which does not possess those alteration(s). In some cases, although rare, it may be desirable to decrease the affinity of an antibody to its antigen, but this is generally not preferred.

Affinity maturation can be done to increase the binding affinity of the antibody for the antigen by at least about 10% to 50-100-150% or more, or from 1 to 5 fold as compared to the “parent” antibody. Preferred affinity matured antibodies will have nanomolar or even picomolar affinities for the target antigen. Affinity matured antibodies are produced by known procedures. See, for example, Marks et al., 1992, Biotechnology 10:779-783 that describes affinity maturation by variable heavy chain (VH) and variable light chain (VL) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

Alternatively, amino acid modifications can be made in one or more of the CDRs of the antibodies of the invention that are “silent”, e.g. that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs of 2.43 and YTS169. In addition, as outlined below, amino acid modifications can also independently and optionally made in any region outside the CDRs, including framework and constant regions.

In some embodiments, the anti-CD8 antibodies of the invention are composed of a variant Fc domain. As is known in the art, the Fc region of an antibody interacts with a number of Fc receptors and ligands, imparting an array of important functional capabilities referred to as effector functions. Suitable modifications can be made at one or more positions as is generally outlined, for example in U.S. patent application Ser. No. 11/841,654 and references cited therein, US 2004/013210, US 2005/0054832, US 2006/0024298, US 2006/0121032, US 2006/0235208, US 2007/0148170, U.S. Ser. No. 12/341,769, U.S. Pat. No. 6,737,056, U.S. Pat. No. 7,670,600, U.S. Pat. No. 6,086,875 all of which are expressly incorporated by reference in their entirety, and in particular for specific amino acid substitutions that increase binding to Fc receptors.

In addition to the modifications outlined above, other modifications can be made. For example, the molecules may be stabilized by the incorporation of disulphide bridges linking the VH and VL domains (Reiter et al., 1996, Nature Biotech. 14:1239-1245, entirely incorporated by reference). In addition, there are a variety of covalent modifications of antibodies that can be made as outlined below.

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues may also be derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole and the like.

In addition, modifications at cysteines are particularly useful in antibody-drug conjugate (ADC) applications, further described below. In some embodiments, the constant region of the antibodies can be engineered to contain one or more cysteines that are particularly “thiol reactive”, so as to allow more specific and controlled placement of the drug moiety. See for example U.S. Pat. No. 7,521,541, incorporated by reference in its entirety herein.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using 125I or 131I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking antibodies to a water-insoluble support matrix or surface for use in a variety of methods, in addition to methods described below. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cynomolgusogen bromide-activated carbohydrates and the reactive substrates described in U.S. Pat. Nos. 3,969,287; 3,691,016; 4,195,128; 4,247,642; 4,229,537; and 4,330,440, all entirely incorporated by reference, are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983], entirely incorporated by reference), acetylation of the N-terminal amine, and amidation of any C-terminal carboxyl group.

In addition, as will be appreciated by those in the art, labels (including fluorescent, enzymatic, magnetic, radioactive, etc. can all be added to the antibodies (as well as the other compositions of the invention)).

Another type of covalent modification is alterations in glycosylation. In another embodiment, the antibodies disclosed herein can be modified to include one or more engineered glycoforms. By “engineered glycoform” as used herein is meant a carbohydrate composition that is covalently attached to the antibody, wherein said carbohydrate composition differs chemically from that of a parent antibody. Engineered glycoforms may be useful for a variety of purposes, including but not limited to enhancing or reducing effector function. A preferred form of engineered glycoform is afucosylation, which has been shown to be correlated to an increase in ADCC function, presumably through tighter binding to the FcγRIIIa receptor. In this context, “afucosylation” means that the majority of the antibody produced in the host cells is substantially devoid of fucose, e.g. 90-95-98% of the generated antibodies do not have appreciable fucose as a component of the carbohydrate moiety of the antibody (generally attached at N297 in the Fc region). Defined functionally, afucosylated antibodies generally exhibit at least a 50% or higher affinity to the FcγRIIIa receptor.

Engineered glycoforms may be generated by a variety of methods known in the art (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473; U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1, all entirely incorporated by reference; (Potelligent® technology [Biowa, Inc., Princeton, N.J.]; GlycoMAb® glycosylation engineering technology [Glycart Biotechnology AG, Zurich, Switzerland]). Many of these techniques are based on controlling the level of fucosylated and/or bisecting oligosaccharides that are covalently attached to the Fc region, for example by expressing an IgG in various organisms or cell lines, engineered or otherwise (for example Lec-13 CHO cells or rat hybridoma YB2/0 cells, by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the IgG has been expressed. For example, the “sugar engineered antibody” or “SEA technology” of Seattle Genetics functions by adding modified saccharides that inhibit fucosylation during production; see for example 20090317869, hereby incorporated by reference in its entirety. Engineered glycoform typically refers to the different carbohydrate or oligosaccharide; thus an antibody can include an engineered glycoform.

Alternatively, engineered glycoform may refer to the IgG variant that comprises the different carbohydrate or oligosaccharide. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the antibody is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330 and in Aplin and Wriston, 1981, CRC Crit. Rev. Biochem., pp. 259-306, both entirely incorporated by reference.

Removal of carbohydrate moieties present on the starting antibody (e.g. post-translationally) may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131, both entirely incorporated by reference. Enzymatic cleavage of carbohydrate moieties on polypeptides can be achieved by the use of a variety of endo- and exo-glycosidases as described by Thotakura et al., 1987, Meth. Enzymol. 138:350, entirely incorporated by reference. Glycosylation at potential glycosylation sites may be prevented by the use of the compound tunicamycin as described by Duskin et al., 1982, J. Biol. Chem. 257:3105, entirely incorporated by reference. Tunicamycin blocks the formation of protein-N-glycoside linkages.

Another type of covalent modification of the antibody comprises linking the antibody to various nonproteinaceous polymers, including, but not limited to, various polyols such as polyethylene glycol, polypropylene glycol or polyoxyalkylenes, in the manner set forth in, for example, 2005-2006 PEG Catalog from Nektar Therapeutics (available at the Nektar website) U.S. Pat. Nos. 4,640,835; 4,496,689; 4,301,144; 4,670,417; 4,791,192 or 4,179,337, all entirely incorporated by reference. In addition, as is known in the art, amino acid substitutions may be made in various positions within the antibody to facilitate the addition of polymers such as PEG. See for example, U.S. Publication No. 2005/0114037A1, entirely incorporated by reference.

The present invention provides a number of antibodies each with a specific set of CDRs (including, as outlined above, some amino acid substitutions). As outlined above, the antibodies can be defined by sets of 6 CDRs, by variable regions, or by full-length heavy and light chains, including the constant regions. In addition, as outlined above, amino acid substitutions may also be made. In general, in the context of changes within CDRs, due to the relatively short length of the CDRs, the amino acid modifications are generally described in terms of the number of amino acid modifications that may be made. While this is also applicable to the discussion of the number of amino acid modifications that can be introduced in variable, constant or full length sequences, in addition to number of changes, it is also appropriate to define these changes in terms of the “% identity”. Thus, as described herein, antibodies included within the invention are 80, 85, 90, 95, 98 or 99% identical to 2.43 or YTS169 described herein.

In some embodiments, antibodies that compete with the antibodies of the invention (for example, with 2.43 or YTS169) for binding to human CD8 and/or murine CD8 are provided. Competition for binding to CD8 or a portion of CD8 by two or more anti-CD8 antibodies may be determined by any suitable technique, as is known in the art.

Competition in the context of the present invention refers to any detectably significant reduction in the propensity of an antibody of the invention (e.g., 2.43 or YTS169) to bind its particular binding partner, e.g. CD8, in the presence of the test compound. Typically, competition means an at least about 10-100% reduction in the binding of an antibody of the invention to CD8 in the presence of the competitor, as measured by standard techniques such as ELISA or Biacore® assays. Thus, for example, it is possible to set criteria for competitiveness wherein at least about 10% relative inhibition is detected; at least about 15% relative inhibition is detected; or at least about 20% relative inhibition is detected before an antibody is considered sufficiently competitive. In cases where epitopes belonging to competing antibodies are closely located in an antigen, competition may be marked by greater than about 40% relative inhibition of CD8 binding (e.g., at least about 45% inhibition, such as at least about 50% inhibition, for instance at least about 55% inhibition, such as at least about 60% inhibition, for instance at least about 65% inhibition, such as at least about 70% inhibition, for instance at least about 75% inhibition, such as at least about 80% inhibition, for instance at least about 85% inhibition, such as at least about 90% inhibition, for instance at least about 95% inhibition, or higher level of relative inhibition).

In some cases, one or more of the components of the competitive binding assays are labeled.

It may also be the case that competition may exist between anti-CD8 antibodies with respect to more than one of CD8 epitope, and/or a portion of CD8, e.g. in a context where the antibody-binding properties of a particular region of CD8 are retained in fragments thereof, such as in the case of a well-presented linear epitope located in various tested fragments or a conformational epitope that is presented in sufficiently large CD8 fragments as well as in CD8.

Assessing competition typically involves an evaluation of relative inhibitory binding using an antibody of the invention, CD8 (either human or murine or both), and the test molecule. Test molecules can include any molecule, including other antibodies, small molecules, peptides, etc. The compounds are mixed in amounts that are sufficient to make a comparison that imparts information about the selectivity and/or specificity of the molecules at issue with respect to the other present molecules.

The amounts of test compound, CD8 and antibodies of the invention may be varied. For instance, for ELISA assessments about 5-50 μg (e.g., about 10-50 μg, about 20-50 μg, about 5-20 μg, about 10-20 μg, etc.) of the anti-CD8 antibody and/or CD8 targets are required to assess whether competition exists. Conditions also should be suitable for binding. Typically, physiological or near-physiological conditions (e.g., temperatures of about 20-40° C., pH of about 7-8, etc.) are suitable for anti-CD8:CD8 binding.

Often competition is marked by a significantly greater relative inhibition than about 5% as determined by ELISA and/or FACS analysis. It may be desirable to set a higher threshold of relative inhibition as a criteria/determinant of what is a suitable level of competition in a particular context (e.g., where the competition analysis is used to select or screen for new antibodies designed with the intended function of blocking the binding of another peptide or molecule binding to CD8 (e.g., the natural binding partners of CD8 or naturally occurring anti-CD8 antibody).

In some embodiments, the anti-CD8 antibody of the present invention specifically binds to one or more residues or regions in CD8 but also does not cross-react with other proteins with homology to CD8.

Typically, a lack of cross-reactivity means less than about 5% relative competitive inhibition between the molecules when assessed by ELISA and/or FACS analysis using sufficient amounts of the molecules under suitable assay conditions.

The disclosed antibodies may find use in blocking a ligand-receptor interaction or inhibiting receptor component interaction. The anti-CD8 antibodies of the invention may be “blocking” or “neutralizing.” A “neutralizing antibody” is intended to refer to an antibody whose binding to CD8 results in inhibition of the biological activity of CD8, for example its capacity to interact with ligands, enzymatic activity, and/or signaling capacity Inhibition of the biological activity of CD8 can be assessed by one or more of several standard in vitro or in vivo assays known in the art.

Inhibits binding” or “blocks binding” (for instance when referring to inhibition/blocking of binding of a CD8 binding partner to CD8) encompass both partial and complete inhibition/blocking. The inhibition/blocking of binding of a CD8 binding partner to CD8 may reduce or alter the normal level or type of cell signaling that occurs when a CD8 binding partner binds to CD8 without inhibition or blocking Inhibition and blocking are also intended to include any measurable decrease in the binding affinity of a CD8 binding partner to CD8 when in contact with an anti-CD8 antibody, as compared to the ligand not in contact with an anti-CD8 antibody, for instance a blocking of binding of a CD8 binding partner to CD8 by at least about 10%, 20%, 30%, 40%, 50%, 60%, 70%, 80%, 90%, 99%, or 100%.

The present invention further provides methods for producing the disclosed anti-CD8 antibodies. These methods encompass culturing a host cell containing isolated nucleic acid(s) encoding the antibodies of the invention. As will be appreciated by those in the art, this can be done in a variety of ways, depending on the nature of the antibody. In some embodiments, in the case where the antibodies of the invention are full length traditional antibodies, for example, a heavy chain variable region and a light chain variable region under conditions such that an antibody is produced and can be isolated.

In general, nucleic acids are provided that encode the antibodies of the invention. Such polynucleotides encode for both the variable and constant regions of each of the heavy and light chains, although other combinations are also contemplated by the present invention in accordance with the compositions described herein. The present invention also contemplates oligonucleotide fragments derived from the disclosed polynucleotides and nucleic acid sequences complementary to these polynucleotides.

The polynucleotides can be in the form of RNA or DNA. Polynucleotides in the form of DNA, cDNA, genomic DNA, nucleic acid analogs, and synthetic DNA are within the scope of the present invention. The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence, which sequence, as a result of the redundancy or degeneracy of the genetic code, encodes the same polypeptides as the DNA provided herein.

In some embodiments, nucleic acid(s) encoding the antibodies of the invention are incorporated into expression vectors, which can be extrachromosomal or designed to integrate into the genome of the host cell into which it is introduced. Expression vectors can contain any number of appropriate regulatory sequences (including, but not limited to, transcriptional and translational control sequences, promoters, ribosomal binding sites, enhancers, origins of replication, etc.) or other components (selection genes, etc.), all of which are operably linked as is well known in the art. In some cases two nucleic acids are used and each put into a different expression vector (e.g. heavy chain in a first expression vector, light chain in a second expression vector), or alternatively they can be put in the same expression vector. It will be appreciated by those skilled in the art that the design of the expression vector(s), including the selection of regulatory sequences may depend on such factors as the choice of the host cell, the level of expression of protein desired, etc.

In general, the nucleic acids and/or expression can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g. in the presence of an inducer, in a suitable non-human animal, in suitable culture media supplemented with appropriate salts, growth factors, antibiotics, nutritional supplements, etc.), whereby the encoded polypeptide(s) are produced. In some cases, the heavy chains are produced in one cell and the light chain in another.

Mammalian cell lines available as hosts for expression are known in the art and include many immortalized cell lines available from the American Type Culture Collection (ATCC), Manassas, Va. including but not limited to Chinese hamster ovary (CHO) cells, HEK 293 cells, NSO cells, HeLa cells, baby hamster kidney (BHK) cells, monkey kidney cells (COS), human hepatocellular carcinoma cells (e.g., Hep G2), and a number of other cell lines. Non-mammalian cells including but not limited to bacterial, yeast, insect, and plants can also be used to express recombinant antibodies. In some embodiments, the antibodies can be produced in transgenic animals such as cows or chickens.

### Pharmacokinetics of Antibodies In Vivo

Several factors are involved in the pharmacokinetics of antibody therapy and antibody-based imaging. One variable among patients is the antigen burden. This is reflected in, for example, the density of CD8 molecules on the cell surface. The native expression of CD8 on the cell surface is termed the “antigen sink.” This antigen sink will ‘sop up’ anti-CD8 antibodies, resulting in lower serum antibody levels and more rapid clearance.

Regardless of whether CD8 undergoes rapid modulation, all surface molecules turn over at some rate. The clearance of antibody from the serum, in part determined by antibody levels, is a complex process that may change owing to variable receptor expression patterns. Patients with accumulating antibody levels have a much higher response rate compared with patients who have a rapid clearance. However, other issues such as disease (e.g., tumor type and tumor burden) complicate this analysis further.

Clinical trials known to one of skill in the art based on measured serum antibody levels can be used to answer the question of whether higher doses or more frequent antibody administration would result in higher response rates for patients with unfavorable pharmacokinetics. It is unlikely that repeated administration of standard dosing will affect response rate in nonresponding patients with poor pharmacokinetics. Conversely, repeated dosing of an antibody may prolong time to progression in responding patients.

As applied herein, analysis of CD8 antibody sinks can be used to determine the optimal amount of CD8 minibodies and diabodies that should be administered to a patient as an imaging and/or therapeutic agent.

### Diagnostic and Prognostic Methods

In certain aspects, the present invention provides methods of diagnosing or providing a prognosis for a CD8 mediated disease, e.g., a cancer that overexpresses CD8, such as prostate, bladder, or pancreatic cancer. As used herein, the term “providing a prognosis” refers to providing a prediction of the probable course and outcome of a cancer or the likelihood of recovery from the CD8 mediated disease. In certain instances, patients with a CD8 mediated disease have negative or low CD8 expression and have a longer disease-specific survival as compared to those with high CD8 expression. As such, the level of CD8 expression can be used as a prognostic indicator, with negative or low expression as an indication of a good prognosis, e.g., a longer disease-specific survival.

The methods of the present invention can also be useful for diagnosing the severity of a CD8 mediated disease, e.g., a cancer that overexpresses CD8. As a non-limiting example, the level of CD8 expression can be used to determine the stage or grade of a CD8 mediated disease such as cancer, e.g., according to the Tumor/Nodes/Metastases (TNM) system of classification (International Union Against Cancer, 6th edition, 2002) or the Whitmore-Jewett staging system (American Urological Association). Typically, cancers are staged using a combination of physical examination, blood tests, and medical imaging. If tumor tissue is obtained via biopsy or surgery, examination of the tissue under a microscope can also provide pathologic staging. In certain instances, cancer patients with high CD8 expression have a more severe stage or grade of that type of cancer. As such, the level of CD8 expression can be used as a diagnostic indicator of the severity of a cancer or of the risk of developing a more severe stage or grade of the cancer. In certain other instances, the stage or grade of a cancer assists a practitioner in determining the prognosis for the cancer and in selecting the appropriate cancer therapy.

The diagnostic and prognostic methods of the present invention advantageously utilize novel engineered humanized antibody fragments that bind to cell surface CD8. Such antibody fragments can be used to determine a level of CD8 expression in tumor tissue or cancerous cells and then compared to a baseline value or range. Typically, the baseline value is representative of CD8 expression levels in a healthy person not suffering from a CD8 mediated disease. Variation of CD8 levels from the baseline range (i.e., either up or down) indicates that the subject has a CD8 mediated disease or is at risk of developing a CD8 mediated disease. In some embodiments, the level of CD8 expression is measured by taking a blood, urine, prostatic fluid, or tumor tissue sample from a subject and measuring the amount of CD8 in the sample using any number of detection methods known in the art. For example, a pull-down assay can be performed on samples such as serum or prostatic fluid using the CD8 antibody fragments described herein coupled to magnetic beads (e.g., Dynabeads®; Invitrogen Corp., Carlsbad, Calif.) to determine the level of CD8 expression. In other embodiments, the CD8 expression is measured in vivo with labeled (e.g., radiolabeled) anti-CD8 antibodies.

In some embodiments, the expression of CD8 in a cancerous or potentially cancerous tissue may be evaluated by visualizing the presence and/or localization of CD8 in the subject. Any technique known in the art for visualizing tumors, tissues, or organs in live subjects can be used in the imaging methods of the present invention. Preferably, the in vivo imaging of cancerous or potentially cancerous tissue is performed using an antibody fragment that binds to the surface of cells expressing CD8, wherein the CD8 antibody fragment is linked to an imaging agent such as a detectable moiety (i.e., a contrast agent). A detectable moiety can be coupled either directly or indirectly to the CD8 antibody fragment described herein using methods well known in the art. A wide variety of detectable moieties can be used, with the choice of label depending on the sensitivity required, ease of conjugation with the CD8 antibody fragments, stability requirements, and available instrumentation and disposal provisions. Suitable detectable moieties include, but are not limited to, radionuclides as described above, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

The detectable moiety can be visualized in a subject using any device or method known in the art. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a detectable moiety linked to a CD8 antibody fragment of the present invention. Positron Emission Tomography (PET) is another suitable technique for detecting radiation in a subject to visualize tumors in living patients according to the methods of the present invention. Furthermore, U.S. Pat. No. 5,429,133 describes a laparoscopic probe for detecting radiation concentrated in solid tissue tumors. Miniature and flexible radiation detectors intended for medical use are produced by Intra-Medical LLC, Santa Monica, Calif. Magnetic Resonance Imaging (MRI) or any other imaging technique known to one of skill in the art (e.g., radiography (i.e., X-rays), computed tomography (CT), fluoroscopy, etc.) is also suitable for detecting the radioactive emissions of radionuclides.

Various in vivo optical imaging techniques that are suitable for the visualization of fluorescent and/or enzymatic and/or radio-labels or markers include, but are not limited to, fluorescence microendoscopy (see, e.g., Flusberg et al., Optics Lett., 30:2272-2274 (2005)), fiber-optic fluorescence imaging (see, e.g., Flusberg et al., Nature Methods, 2:941-950 (2005)), fluorescence imaging using a flying-spot scanner (see, e.g., Ramanujam et al., IEEE Trans. Biomed. Eng., 48:1034-1041 (2001)), catheter-based imaging systems (see, e.g., Funovics et al., Radiology, 231:659-666 (2004)), near-infrared imaging systems (see, e.g., Mahmood et al., Radiology, 213:866-870 (1999)), fluorescence molecular tomography (see, e.g., Gurfinkel et al., Dis. Markers, 19:107-121 (2004)), and bioluminescent imaging (see, e.g., Dikmen et al., Turk. J. Med. Sci., 35:65-70 (2005)).

The CD8 antibody fragments of the present invention, when conjugated to any of the above-described detectable moieties, can be administered in doses effective to achieve the desired image of tumor tissue or cancerous cells or CD8 mediated disease tissue in a subject. Such doses may vary widely, depending upon the particular detectable label employed, the type of CD8 mediated disease the patient is inflicted with, the imaging equipment being used, and the like. However, regardless of the detectable moiety or imaging technique used, such detection is aimed at determining where the CD8 antibody fragment is concentrated in a subject, with such concentration being an indicator of the location of a tumor or tumor cells or cells that are affected by the CD8 mediated disease. In certain embodiments, such detection is aimed at determining the extent of tumor regression in a subject, with the size of the tumor being an indicator of the efficacy of cancer therapy. In certain embodiments, such detection is aimed at determining the extent of the CD8 mediated disease in a subject.

In certain embodiments, the in vivo imaging can be performed 4 hours after administration of the anti-CD8 antibody. In certain embodiments, the in vivo imaging can be performed 30 min, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, or 10 or more hours after administration of the anti-CD8 antibody. In certain embodiments, the in vivo imaging can be performed 10 hours, or 11 hours, or 12 hours, or 13 hours, or 14 hours, or 15 hours, or 16 hours, or 17 hours, or 18 hours, or 19 hours, or 20 hours, or 21 hours, or 22 hours, or 23 hours, or 1 day after administration of the anti-CD8 antibody. In certain embodiments, the in vivo imaging can be performed 1 day or 2 days or 3 days or 4 days or 5 days or 6 days or 7 days or 8 days or 10 days of 11 days or 12 days or 13 days or 14 days after administration of the anti-CD8 antibody. In ceratin embodiments, the in vivo imaging can be performed in any combination of the above-described time increments. In specific embodiments, the anti-CD8 antibody is administered by injection or IV perfusion.

### Methods of Administration and Diagnostic and Pharmaceutical Compositions

As described herein, antibody fragments that bind to CD8 on the surface of cells such as cancer cells, for example, are particularly useful in treating, imaging, diagnosing, and/or providing a prognosis for CD8 mediated diseases (e.g., cancer). For therapeutic applications, the CD8 antibody fragments of the present invention can be administered alone or co-administered in combination with conventional chemotherapy, radiotherapy, hormonal therapy, and/or immunotherapy.

As a non-limiting example, CD8 antibody fragments can be co-administered with conventional chemotherapeutic agents including alkylating agents (e.g., cisplatin, cyclophosphamide, carboplatin, ifosfamide, chlorambucil, busulfan, thiotepa, nitrosoureas, etc.), anti-metabolites (e.g., 5-fluorouracil, azathioprine, methotrexate, fludarabine, etc.), plant alkaloids (e.g., vincristine, vinblastine, vinorelbine, vindesine, podophyllotoxin, paclitaxel, docetaxel, etc.), topoisomerase inhibitors (e.g., amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, plicamycin, etc.), and the like.

CD8 antibody fragments can also be co-administered with conventional hormonal therapaeutic agents including, but not limited to, steroids (e.g., dexamethasone), finasteride, aromatase inhibitors, tamoxifen, and gonadotropin-releasing hormone agonists (GnRH) such as goserelin.

Additionally, CD8 antibody fragments can be co-administered with conventional immunotherapeutic agents including, but not limited to, immunostimulants (e.g. Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD8 monoclonal antibodies as described herein which are conjugated to ¹¹¹In, ⁹⁰Y, or ¹³¹I, for example).

In a further embodiment, CD8 antibody fragments can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, and/or ²¹²Bi, optionally conjugated to antibodies directed against tumor antigens. Alternatively, compositions of the present invention comprise CD8-specific antibody or fragments or linking groups conjugated thereto that are radiolabeled with a radionuclide such as ¹⁸F, ¹²⁴I, ¹²⁵I, and/or ¹³¹I. In certain other instances, the imaging compositions of the present invention comprise CD8-specific antibody or fragments conjugated to a bifunctional chelating agent that contains a radionuclide such as ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁹⁰Y, ¹¹¹In, ⁹⁹Tc, and/or ²⁰¹Tl bound thereto. Alternatively, the imaging compositions of the present invention comprise anti-CD8 antibody fragments or linking groups conjugated thereto that are radiolabeled with a radionuclide such as ¹⁸F and/or ¹³¹I

In some embodiments, the compositions of the present invention comprise CD8 antibody fragments and a physiologically (i.e., pharmaceutically) acceptable carrier. As used herein, the term “carrier” refers to a typically inert substance used as a diluent or vehicle for a drug such as a therapeutic agent. The term also encompasses a typically inert substance that imparts cohesive qualities to the composition. Typically, the physiologically acceptable carriers are present in liquid, solid, or semi-solid form. Examples of liquid carriers include physiological saline, phosphate buffer, normal buffered saline (135-150 mM NaCl), water, buffered water, 0.4% saline, 0.3% glycine, glycoproteins to provide enhanced stability (e.g., albumin, lipoprotein, globulin, etc.), and the like. Examples of solid or semi-solid carriers include mannitol, sorbitol, xylitol, maltodextrin, lactose, dextrose, sucrose, glucose, inositol, powdered sugar, molasses, starch, cellulose, microcrystalline cellulose, polyvinylpyrrolidone, acacia gum, guar gum, tragacanth gum, alginate, extract of Irish moss, panwar gum, ghatti gum, mucilage of isapol husks, Veegum®, larch arabogalactan, gelatin, methylcellulose, ethylcellulose, carboxymethylcellulose, hydroxypropylmethylcellulose, polyacrylic acid (e.g., Carbopol), calcium silicate, calcium phosphate, dicalcium phosphate, calcium sulfate, kaolin, sodium chloride, polyethylene glycol, and combinations thereof. Since physiologically acceptable carriers are determined in part by the particular composition being administered as well as by the particular method used to administer the composition, there are a wide variety of suitable formulations of pharmaceutical compositions of the present invention (see, e.g., Remington's Pharmaceutical Sciences, 17.sup.th ed., 1989).

The pharmaceutical compositions of the present invention may be sterilized by conventional, well-known sterilization techniques or may be produced under sterile conditions. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents, wetting agents, and the like, eg., sodium acetate, sodium lactate, sodium chloride, potassium chloride, calcium chloride, sorbitan monolaurate, and triethanolamine oleate.

Formulations suitable for oral administration can comprise: (a) liquid solutions, such as an effective amount of a packaged CD8 antibody fragment suspended in diluents, e.g., water, saline, or PEG 400; (b) capsules, sachets, or tablets, each containing a predetermined amount of a CD8 antibody fragment, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise a CD8 antibody fragment in a flavor, e.g., sucrose, as well as pastilles comprising the polypeptide or peptide fragment in an inert base, such as gelatin and glycerin or sucrose and acacia emulsions, gels, and the like, containing, in addition to the polypeptide or peptide, carriers known in the art.

The CD8 antibody fragment of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which comprises an effective amount of a packaged CD8 antibody fragment with a suppository base. Suitable suppository bases include natural or synthetic triglycerides or paraffin hydrocarbons. In addition, it is also possible to use gelatin rectal capsules which contain a combination of the CD8 antibody fragment of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Injection solutions and suspensions can also be prepared from sterile powders, granules, and tablets. In the practice of the present invention, compositions can be administered, for example, by intravenous infusion, orally, topically, intraperitoneally, intravesically, or intrathecally. Parenteral administration, oral administration, and intravenous administration are the preferred methods of administration. The formulations of compounds can be presented in unit-dose or multi-dose sealed containers, such as ampoules and vials.

The pharmaceutical preparation is preferably in unit dosage form. In such form the preparation is subdivided into unit doses containing appropriate quantities of the active component, e.g., a CD8 antibody fragment. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

In therapeutic use for the treatment of cancer, the CD8 antibody fragments utilized in the pharmaceutical compositions of the present invention are administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the CD8 antibody fragment being employed. For example, dosages can be empirically determined considering the type and stage of cancer diagnosed in a particular patient. The dose administered to a patient, in the context of the present invention, should be sufficient to affect a beneficial therapeutic response in the patient over time. The size of the dose will also be determined by the existence, nature, and extent of any adverse side-effects that accompany the administration of a particular CD8 antibody fragment in a particular patient. Determination of the proper dosage for a particular situation is within the skill of the practitioner. Generally, treatment is initiated with smaller dosages which are less than the optimum dose of the CD8 antibody fragment. Thereafter, the dosage is increased by small increments until the optimum effect under circumstances is reached. For convenience, the total daily dosage may be divided and administered in portions during the day, if desired.

Furthermore, the anti-CD8 antibodies described herein can be formulated into an immunogenic composition. In certain embodiments the immunogenic composition is a vaccine. In specific embodiments, the vaccine creates immunity against cancer, autoimmune disease, or viral infections.

### Methods of Treatment

Antibody Compositions for In Vivo Administration

Formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. For example, it may be desirable to provide antibodies with other specificities. Alternatively, or in addition, the composition may comprise a cytotoxic agent, cytokine, growth inhibitory agent and/or small molecule antagonist. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration should be sterile, or nearly so. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and .gamma. ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Administrative Modalities

The antibodies and chemotherapeutic agents of the invention are administered to a subject, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the antibody is preferred.

In certain aspects, the antibodies and chemotherapeutic agents of the invention are administered to a subject with cancer. In certain aspects, the antibodies and chemotherapeutic agents of the invention are administered to a subject with breast cancer, brain cancer, colon cancer, melanoma, leukemia (e.g., AML), pancreatic cancer, prostate cancer, ovarian cancer, lung cancer, and/or gastric cancer.

In certain aspects, the antibodies are administered to a subject with an autoimmune disease. In certain aspects, the antibodies are administered concurrently with autoimmune treatment agents. In certain embodiments, the antibodies are administered before, after, or at the same time as the autoimmune treatment agents.

In certain aspects, the antibodies are administered concurrently with anti-viral agents. In certain embodiments, the antibodies are administered before, after, or at the same time as anti-viral agents. In certain embodiments the antibodies are administered to a subject with malaria or with HIV/AIDS.

Treatment Modalities

In the methods of the invention, therapy is used to provide a positive therapeutic response with respect to a disease or condition. By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling.

In addition to these positive therapeutic responses, the subject undergoing therapy may experience the beneficial effect of an improvement in the symptoms associated with the disease.

Such a response may persist for at least 4 to 8 weeks, or sometimes 6 to 8 weeks, following treatment according to the methods of the invention. Alternatively, an improvement in the disease may be categorized as being a partial response. By “partial response” is intended at least about a 50% decrease in all measurable tumor burden (i.e., the number of malignant cells present in the subject, or the measured bulk of tumor masses or the quantity of abnormal monoclonal protein) in the absence of new lesions, which may persist for 4 to 8 weeks, or 6 to 8 weeks.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the anti-CD8 antibodies used in the present invention depend on the disease or condition to be treated and may be determined by the persons skilled in the art.

An exemplary, non-limiting range for a therapeutically effective amount of an anti-CD8 antibody used in the present invention is about 0.1-100 mg/kg, such as about 0.1-50 mg/kg, for example about 0.1-20 mg/kg, such as about 0.1-10 mg/kg, for instance about 0.5, about such as 0.3, about 1, or about 3 mg/kg. In another embodiment, the antibody is administered in a dose of 1 mg/kg or more, such as a dose of from 1 to 20 mg/kg, e.g. a dose of from 5 to 20 mg/kg, e.g. a dose of 8 mg/kg.

A medical professional having ordinary skill in the art may readily determine and prescribe the effective amount of the pharmaceutical composition required. For example, a physician or a veterinarian could start doses of the medicament employed in the pharmaceutical composition at levels lower than that required in order to achieve the desired therapeutic effect and gradually increase the dosage until the desired effect is achieved.

In one embodiment, the anti-CD8 antibody is administered by infusion in a weekly dosage of from 10 to 500 mg/kg such as from 200 to 400 mg/kg. Such administration may be repeated, e.g., 1 to 8 times, such as 3 to 5 times. The administration may be performed by continuous infusion over a period of from 2 to 24 hours, such as from 2 to 12 hours.

In one embodiment, the anti-CD8 antibody is administered by slow continuous infusion over a long period, such as more than 24 hours, if required to reduce side effects including toxicity.

In one embodiment the anti-CD8 antibody is administered in a weekly dosage of from 250 mg to 2000 mg, such as for example 300 mg, 500 mg, 700 mg, 1000 mg, 1500 mg or 2000 mg, for up to 8 times, such as from 4 to 6 times. The administration may be performed by continuous infusion over a period of from 2 to 24 hours, such as from 2 to 12 hours. Such regimen may be repeated one or more times as necessary, for example, after 6 months or 12 months. The dosage may be determined or adjusted by measuring the amount of compound of the present invention in the blood upon administration by for instance taking out a biological sample and using anti-idiotypic antibodies which target the antigen binding region of the anti-CD8 antibody.

In a further embodiment, the anti-CD8 antibody is administered once weekly for 2 to 12 weeks, such as for 3 to 10 weeks, such as for 4 to 8 weeks.

In one embodiment, the anti-CD8 antibody is administered by maintenance therapy, such as, e.g., once a week for a period of 6 months or more.

In one embodiment, the anti-CD8 antibody is administered by a regimen including one infusion of an anti-CD8 antibody followed by an infusion of an anti-CD8 antibody conjugated to a radioisotope. The regimen may be repeated, e.g., 7 to 9 days later.

As non-limiting examples, treatment according to the present invention may be provided as a daily dosage of an antibody in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

Combination Therapy

In some embodiments the anti-CD8 antibody molecule thereof is used in combination with one or more additional therapeutic agents, e.g. a chemotherapeutic agent. Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and other inhibitors of proteins or enzymes known to be upregulated, over-expressed or activated in cancers, the inhibition of which downregulates cell replication.

In some embodiments, the antibodies of the invention can be used prior to, concurrent with, or after treatment with any of the chemotherapeutic agents described herein or known to the skilled artisan at this time or subsequently.

In some embodiments, the antibodies of the invention can be conjugated to one or more of the chemotherapeutic agents described above or known to one of skill in the art.

Efficacy of Methods Described Herein

In certain aspects of this invention, efficacy of anti-CD8 therapy is measured by decreased serum concentrations of tumor specific markers, increased overall survival time, decreased tumor size, cancer remission, decreased metastasis marker response, and decreased chemotherapy adverse affects.

In certain aspects of this invention, efficacy is measured with companion diagnostic methods and products. Companion diagnostic measurements can be made before, during, or after anti-CD8 treatment.

### Companion Diagnostics

In other embodiments, this disclosure relates to companion diagnostic methods and products. In one embodiment, the companion diagnostic method and products can be used to monitor the treatment of cancer. In a specific embodiment, the companion diagnostic method and products can be used to monitor the treatment of breast cancer. In a specific embodiment, the companion diagnostic method and products can be used to monitor the treatment of an autoimmune disease. In one embodiment, the companion diagnostic method and products can be used to monitor the treatment of brain cancer, colon cancer, melanoma, leukemia (e.g., AML), pancreatic cancer, prostate cancer, ovarian cancer, lung cancer, and/or gastric cancer. In one embodiment, the companion diagnostic method and products can be used to monitor the treatment of viral diseases. In one embodiment, the companion diagnostic method and products can be used to monitor the treatment of retroviral diseases.

In some embodiments, the companion diagnostic methods and products include molecular assays to measure levels of proteins, genes or specific genetic mutations. Such measurements can be used, for example, to predict whether anti-CD8 therapy will benefit a specific individual, to predict the effective dosage of anti-CD8 therapy, to monitor anti-CD8 therapy, adjust anti-CD8 therapy, tailor the anti-CD8 therapy to an individual, and track cancer progression and remission.

In some embodiments, the companion diagnostic can be used to monitor a combination therapy.

In some embodiments, the companion diagnostic can include an anti-CD8 antibody described herein.

In some embodiments, the companion diagnostic can be used before, during, or after anti-CD8 therapy.

### Articles of Manufacture

In other embodiments, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is the antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The present invention also provides kits for carrying out the therapeutic, diagnostic, prognostic, and imaging assays described herein. The kits will typically be comprised of one or more containers containing CD8 antibody fragments that bind to cell surface CD8, e.g., in dehydrated form, with instructions for their rehydration and administration. For example, one container of a kit may hold the dehydrated CD8 antibody fragments and another container may hold a buffer suitable for rehydrating the dry components. Kits can include any of the compositions noted above, and optionally further include additional components such as instructions to practice the desired method, control antibodies, antigens, polypeptides or peptides such as a negative control antibody or a positive control antigen, a robotic armature for mixing kit components, and the like.

### Examples

### Example 1

### Design and Production of the Anti-CD8 Minibodies

Determination of V_{H} and V_{L} Sequences from Parental Hybridomas.

The YTS169 hybridoma was obtained from the Therapeutic Immunology Group at Oxford University, UK and cultured in Iscove's Modified Dulbecco's Medium (IMDM) plus 10% FBS and Pen/Strep (21). The 2.43 hybridoma was obtained from ATCC (TIB-210) and cultured in DMEM plus 10% FBS and Pen/Strep. V_{H} and V_{L} sequences were obtained by RT-PCR using primers published by Dubel, S., et al. (Dubel S. et al., Isolation of IgG antibody Fv-DNA from various mouse and rat hybridoma cell lines using the polymerase chain reaction with a simple set of primers. J Immunol Methods. Sep. 30 1994; 175(1):89-95). For the hybridoma 2.43, the obtained V_{H} and V_{L} sequences were confirmed with trypsin digest-mass spectrometry of the purified parental antibody performed at the UCLA core facility. The YTS169 hybridoma was engineered to antibody fragments without further V_{H} and V_{L} validation.

Specifically, RNA was isolated from hybridomas grown in culture using the Quick-RNA MicroPrep™ according to manufacturer instructions (Zymo Research). Freshly isolated RNA was immediately used for reverse transcription-PCR (RT-PCR) using a combination of primers published in Dubel, S., et al. for the heavy chain FR1 region (Bi3, Bi3b and Bi3c) and the kappa chain FR1 region (Bi6, Bi7 and Bi8). The heavy chain CH1 primer used was 5′-CGG AAT TCA GGG GCC ATG GGA TAG AC (SEQ ID NO.: 31). The kappa chain constant domain primer used was 5′-CGG AAT TCG GAT GGT GGG AAG ATG GA (SEQ ID NO.: 32). RT-PCR was performed using the OneStep RT-PCR kit according to manufacturer instructions (Qiagen) before TAE-agarose gel extraction, ligation using TOPO TA Cloning kit (Invitrogen) and DH5a (Invitrogen) transformation.

Specifically, the OneStep RT-PCR kit was performed according to manufacturer instructions (Qiagen) using 30 min reverse transcription at 50° C. followed by PCR activation and reverse polymerase denaturation for 15 min at 95° C. 35 cycles were performed using 1 min at 94° C. for denaturation, 30 s at 55° C. for annealing and 1 min extensions at 72° C. Colonies were selected for miniprep isolation (Invitrogen) of plasmid DNA for subsequent sequencing at UCLA sequencing core facility. Sequences were analyzed with BLAST for V_{H} or V_{L} homology. Sequences were verified by three identical recovered sequences from at least two different experiments.

RT-PCR products were run on 1% TAE-agarose gels. Bands of the correct size were extracted using the Qiagen Gel extraction kit and ligated using TOPO TA Cloning kit by following supplier's instructions (Invitrogen). DH5a (Invitrogen) were transformed with ligations and the resulting colonies were selected for miniprep isolation (Invitrogen) of plasmid DNA for subsequent sequencing at UCLA sequencing core facility. Sequences were analyzed with BLAST for V_{H} or V_{L} homology. Sequences were verified by three identical recovered sequences from at least two different experiments. For the hybridoma 2.43, the obtained V_{H} and V_{L} sequences were confirmed with trypsin digest-mass spectrometry of the purified parental antibody performed at the UCLA core facility. The YTS 169 hybridoma was engineered to antibody fragments without further V_{H} and V_{L} validation.

Design and Construction of Anti-mCD8 Minibodies.

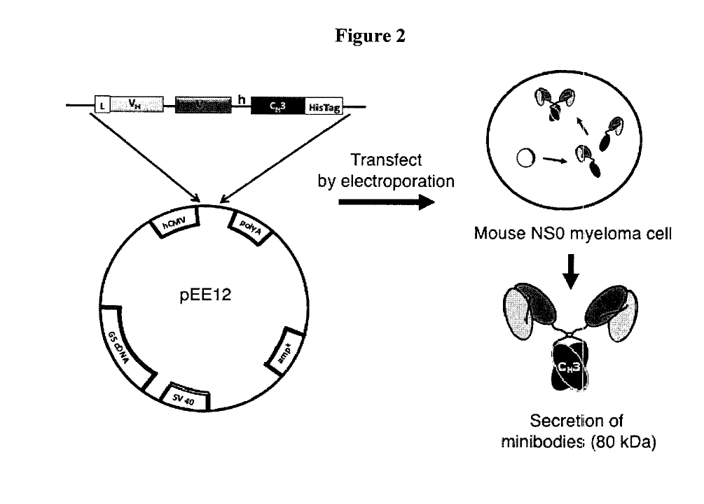

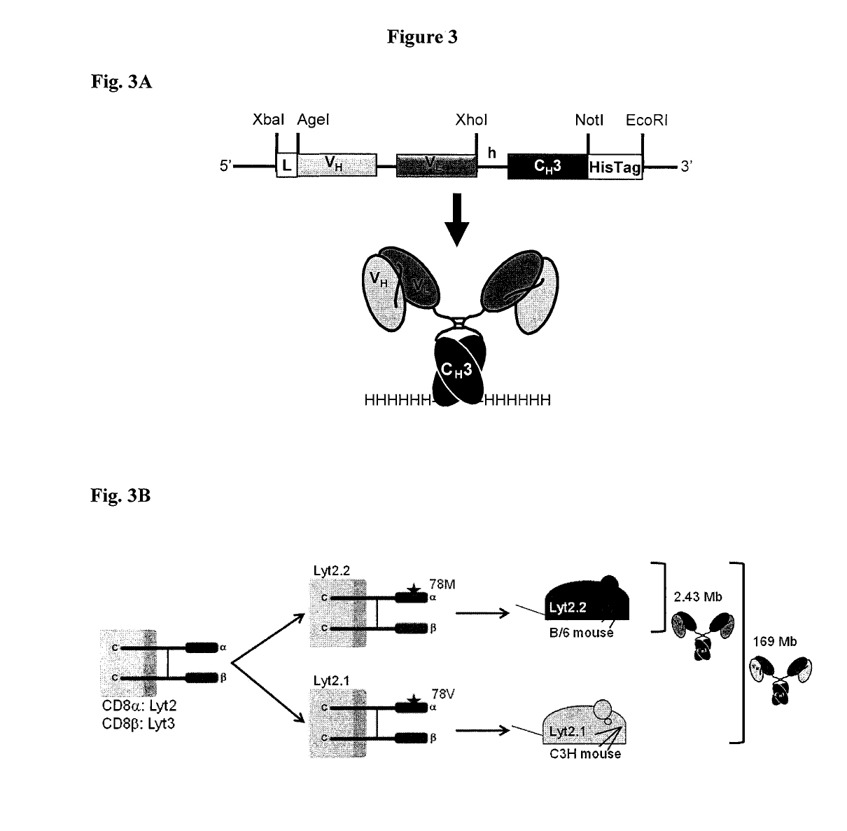

The 2.43 and YTS 169 minibody constructs were synthesized by GeneArt® (Invitrogen, Carlsbad Calif.) to contain a Kozak sequence followed by the mouse Ig kappa secretion signal, V_{H}, an 18 GlySer-rich amino acid linker (GSTSGGGSGGGSGGGGSS (SEQ ID NO: 52)) V_{L}, mouse IgG2a hinge, the mouse IgG2a C_{H}3 domain and a C-terminal hexahistidine sequence (FIGS. 2 and 3). The minibody cassette contains the N-terminal XbaI and C-terminal EcoRI restriction sites for subcloning into the mammalian expression vector pEE12 (Lonza).

Specifically, the full length 2.43 minibody was constructed with the Kozak sequence, followed by the mouse Ig kappa secretion signal, V_{H}, an 18 GlySer-rich amino acid linker, V_{L}, mouse IgG2a hinge, the mouse IgG2a CH3 domain and a C-terminal hexahistidine sequence. The Mb cassette contains the N-terminal XbaI and C-terminal EcoRI restriction sites for subcloning into the mammalian expression vector pEE12.4 (Lonza) using the T4 DNA Ligase (New England Biolabs). An AgeI site was placed at the end secretion sequence directly before the start of the V_{H}. Also, an XhoI site was introduced in between the V_{L} domain and the hinge for subcloning various V_{H}-V_{L} Mb fragments. Finally, a NotI site was place after the CH3 domain before the HisTag sequence (FIGS. 2 and 3). Using this cassette, the V_{H} and V_{L} domains of YTS 169 and YTS 156 hybridomas were synthesized by GeneArt containing the 18-amino acid linker between V_{H} and V_{L} and subcloned into the pEE12-2.43 Mb vector.

Expression and Purification of Engineered Mb Antibody Fragments.

2×10⁶ NS0 mouse myeloma cells were transfected with 10 μg of FspI (New England Biolabs) linearized vector DNA by electroporation (Multiporator, Eppendorf) and selected in glutamine-deficient Dulbecco's modification of Eagle's medium (DMEM; Mediatech, Inc.) as previously described (Yazaki P. J. et al., Mammalian expression and hollow fiber bioreactor production of recombinant anti-CEA diabody and minibody for clinical applications. J Immunol Methods. Jul. 1 2001; 253(1-2):195-208).

The supernatant of individual clones were screened for expression by Ni-NTA HisSorb Plates (Qiagen) using the goat anti-mouse IgG2a-Alkaline Phosphatase conjugate for detection (1:2500, Santa Cruz Biotechnologies). Expression was also confirmed for clones positive for expression by ELISA by sodium dodecyl sulfate polyacrylamide gel electrophoresis (SDS-PAGE) followed by western blot analysis using the same goat anti-mouse IgG2a-Alkaline Phosphatase conjugate (1:2500) and developed with the NBT/BCIP Color Development substrate kit (Promega). The highest producing clones were expanded and brought to terminal culture for expression of secreted proteins. 3-4 week terminal culture was performed in culture media plus 2% FBS.

More specifically, soluble minibodies were purified from cell culture supernatants using NiNTA affinity chromatography (GE Healthcare) using an AKTA purifier FPLC. Supernatants were loaded onto the column in the presence of 10 mM imidazole and eluted with a gradient of 10 to 500 mM imidazole. The purified proteins were then dialyzed against PBS using Slide-A-Lyzer dialysis cassettes (MWCO: 10,000, Thermo Scientific) and concentrated with a Vivaspin 20 (MWCO: 10,000, Sartorius). Final protein concentrations were determined by measuring UV absorbance at 280 nm. Purified proteins were analyzed by SDS-PAGE under non-reducing conditions. Native structural size was determined by Superdex 200 size exclusion column (Pharmacia) using PBS as the running buffer.

Production of Soluble CD8ab Fusion Protein.

PCR was used to amplify the soluble domains of both CD8a (residues Lys1-Ser124 of mature CD8a, Lyt2.2⁺) and CD8β (residues Leu1-Val117 of mature CD8β) and fuse them via a 29 amino acid alpha helical linker (AGSADDARKDAGSKDDARKDDARKDGSSA (SEQ ID NO.: 33)). This linker is similar to the linker previously described for a sCD8αβ fusion (Chang H. C. et al., Structural and mutational analyses of a CD8alphabeta heterodimer and comparison with the CD8alphaalpha homodimer. Immunity. December 2005; 23(6):661-671) except for the GS amino acids at linker positions 12 and 13 (underlined) due to the insertion of a BamHI site for cloning purposes.

Specifically, CD8a was amplified to contain an N-terminal AgeI site and a C-terminal sequence corresponding to AGSADDARKDAGS (SEQ ID NO: 53). CD8β was amplified to contain the N-terminal amino acid sequence GSKDDARKDDARKDGSSA (SEQ ID NO: 54) and a C-terminal NotI site. The following primers were used:

sCD8a-F (SEQ ID NO.: 34):
5′-CACACAGAGCTCACCGGTAAGCCACAGGCACCCGAAC
sCD8αβR (SEQ ID NO.: 35):
5′-TGTGTGGGATCCCGCATCCTTTCTTGCATCGTCGGCAGATC
CTGCAGAGTTCACTTTCTGAAG
sCD8b-F (SEQ ID NO.: 36):
5′-CACACAGGATCCAAAGATGACGCAAGGAAGGACGATGCT
AGGAAGGATGGATCTTCCGCACTCATTCAGACCCCTTCG
sCD8b-R (SEQ ID NO.: 37):
5′-TGTGTGTCTAGACGCGGCCGCAACCACAGTCAGCTTCGTC

PCR products of CD8α and CD8β were gel purified, digested with BamHI and ligated using T4 DNA Ligase. Ligations were used as the template for PCR using the sCD8α-F and sCD8β-R primers (equimolar amounts were ligated for 30 min at room temperature using T4 DNA Ligase). The PCR product was restriction enzyme digested with AgeI and NotI and ligated into the pre-digested pEE12-2.43 Mb cassette constructed above for the addition of a C-terminal HisTag. Sequence verification, NS0 electroporation, clonal selection, protein production and NiNTA purification was performed as described above. Size exclusion chromatography using a Superdex75 column was required for complete purification.

More specifically, Ligations were gel purified and isolated for the correct size and subsequently used as the template for PCR using the sCD8a Forward and sCD8b Reverse NotI primers. The PCR product of the correct size was gel isolated from 1% agarose TAE gel, restriction enzyme digested with AgeI and NotI, gel isolated again and ligated into the pre-digested pEE12-2.43 Mb cassette constructed above for the addition of a C-terminal HisTag. Sequence was verified at UCLA sequencing core facility. NS0 electroporation, selection and protein production and NiNTA purification was performed as described above. Size exclusion chromatography using a Superdex75 column was required for complete purification. sCD8ab was analyzed for binding to purified Mbs size exclusion chromatography with a Superdex200 column on an AKTA purifier and monitoring elution profiles.

Affinity Measurements.

Surface Plasmon Resonance (SPR) analysis was performed on a Biacore 3000 (Precision Antibody, Inc.). Briefly, Mbs were captured using goat anti-mouse IgG Fc and sCD8αβ antigen was flowed over the chip at 100, 50, 25, 12.5, 6.25 and 0 nM. The equilibrium constant (K_{D}) was calculated from the observed on (kₐ) and off (k_{d}) rates.

FITC Conjugation.

The 2.43 Mb was incubated with a 40-fold molar excess of fluorescein isothiocyanate (FITC) at pH 8.5 for 4 hours at 4° C. Excess FITC was removed using a PD-10 column in PBS (GE Healthcare). Protein was concentrated using a 0.5 mL spin filter (MWCO 3 kDa; Amicon) and the conjugation efficiency was evaluated using the NanoDrop (Thermo Scientific) to calculate the ratio of moles fluorescein to moles protein.

Flow Cytometry.

2×10⁵ BW58, TK-1 or EL4 cells in 200 μL PBS plus 1% FBS were incubated with 2 μg of the anti-CD8 Mb constructs for 45 min on ice. After two washes in PBS plus 1% FBS, they were subsequently stained with mouse goat anti-mouse IgG2a-PE (Santa Cruz Biotechnologies) for 45 min on ice in PBS plus 1% FBS. Flow cytometry analysis was performed after two more washes in PBS plus 1% FBS using a BD Biosciences LSR II at the UCLA flow cytometry core facility.

Flow cytometry on primary cells from Lyt2.2+B/6 and Lyt2.1+C3H mice was performed on single cell suspensions from the spleen, bone marrow, peripheral blood, thymus and lymph nodes. Mashing organs over 45 mm and then 30 mm filters in RPMI made single cell suspensions. After red blood cell lysis using ACK (Ammonium-Chloride-Potassium) lysis buffer, the cells were stained for one hour on ice with either Lyt2.2-specific fluorescein conjugated 2.43 Mb or anti-CD8-fluorescein (clone 53-6.7; eBioscience) and the following: anti-CD4-PE (clone GK1.5; eBioscience), anti-CD45-APC (clone 30-F11; eBioscience). Cells were then washed with PBS and analyzed using a BD FACSCanto.

The YTS 105 and YTS 165 hybridomas were also analyzed using the above-described methods.

### Example 2

### In Vivo Preparation and Administration of Anti-CD8 Minibodies

CD8 depletion. Mice were treated for three consecutive days with 330 μg of anti-CD8 depleting antibody (Clone 53-6.7 purchased from UCSF Monoclonal Antibody Core) injected intraperitoneally (165 μL in saline) or 250 μg of 2.43 minibody injected intravenously (125 μL in saline). Two to three days post-treatment, single cell suspensions from the spleen, peripheral blood, thymus and lymph nodes were isolated and stained as described above for CD8 depletion analysis.

SCN-NOTA Conjugation.

All solutions were made metal-free (MF) using Chelex 100 (1.2 g/L; BioRad). The 2.43 and YTS169 Mbs were dialyzed against MF-PBS overnight using Slide-A-Lyzer MINI dialysis units (Thermo Scientific). Proteins at 1-2 mg/mL were then incubated with an 80-fold molar excess of S-2-(4-Isothiocyanatobenzyl)-1,4,7-triazacyclononane-1,4,7-triacetic acid (p-SCN-Bn-NOTA; Macrocyclics) for 4 hours at 4° C. pH was adjusted to 8.5 using 1 M MF-NaOH. Excess p-SCN-Bn-NOTA was removed by PD-10 desalting columns that were pre-equilibrated MF-PBS. Eluted protein was concentrated with Amicon Ultra centrifugal fliters (0.5 mL and 10 kDa MWCO, Milipore) that had been washed twice with MF-PBS.

⁶⁴Cu Radiolabeling.

[⁶⁴Cu]CuCl₂ was obtained from Washington University School of Medicine, Division of Radiological Sciences, St. Louis, Mo.). 5 μL of 250 mM ammonium acetate pH 7.0 was added to ˜37 MBq (˜1 mCi) ⁶⁴CuCl₂ before the addition of 60-80 μg NOTA conjugated minibody and 1.4 mg/mL in saline. Protein was incubated for 30-45 min. at 42° C. before a challenge of 5 mM EDTA for 5 min. at room temperature. Radiolabeling efficiency was measured with ITLC (Biodex medical systems) using saline as the mobile phase. The ITLC strip was cut in half and sections were counted using a Wizard 3″ 1480 Automatic Gamma Counter (Perkin-Elmer). Radiochemical purity was assessed by ITLC using saline as the mobile phase. Protein was purified using BioRad6 Spin columns equilibrated with PBS if the radiochemical purity was <95%.

The percent of functional ⁶⁴Cu-NOTA Mb post-radiolabeling was measured by incubating 30-50 ng radiolabeled Mb with >40×10⁶ antigen-positive (BW58) or antigen-negative (EL-4) murine lymphoma cells in PBS plus 1% FBS for one hour. The amount of activity in supernatant versus the pellet was counted in a gamma counter and the immuno-reactive fraction was calculated as (% cell bound activity/total activity)*100.

MicroPET Imaging.

200 μL doses containing 2.6-2.9 MBq (70-80 μCi, 8-10 μCi/μg) ⁶⁴Cu radiolabeled Mb were prepared in saline and injected i.v. into B/6, C3H or NOD scid gamma (NSG) mice (The Jackson Laboratory). At 4 h post-injection, mice were anesthetized using 2% isoflurane and microPET scans were acquired using an Inveon microPET scanner (Siemens) followed by microCT scan (ImTek). MicroPET images were reconstructed using non-attenuation or scatter corrected filtered back projection and AMIDE was used for image analysis and display.

Biodistribution.

After microPET/CT imaging, mice were euthanized, the organs and blood were collected, weighed and the activity was gamma counted. The percent-injected dose per gram tissue (% ID/g) was calculated using a standard containing two percent of the injected dose. Left and right axillary lymph nodes were pooled and counted for biodistribution studies.

Data Analysis.

Data values are reported as mean±SD. Statistical analysis was performed using a two-tailed Student t-test at the 95% confidence level (p<0.05). MicroPET/CT images are displayed as 20 or 2 mm maximum intensity projections for coronal or transverse images, respectively.

### Example 3

### Production of Anti-CD8 Antibodies

Sequencing Variable Regions of Parental Rat Anti-Murine CD8 Antibodies.

RT-PCR was repeated until at least 2 individual experiments produced three replicates of the same sequence for the V_{H} and V_{L} domains for each hybridoma. For sequence validation of hybridoma 2.43, the V_{H} and V_{L} from RT-PCR sequences were confirmed with tryptic digest-mass spectrometry of the parental antibody. V_{H} amino acid coverage was 35% (41/117) including the complete CDR1 and half of CDR2 and V_{L} amino acid coverage was 62% (66/107) including both CDR2 and CDR3. For YTS169, no sequence verification was performed.

For the hybridomas YTS 105 and YTS 156, the sequences were compared to the crystal structures of the Fab fragment in complex with either sCD8a or sCD8β deposited with PDB ID codes 2ARJ and 3B9K, respectively. RT-PCR sequences for YTS 105 were identical to the published crystal structure except for a few mutations at the 5′ and 3′ terminal ends due to the degenerate primers. RT-PCR sequences obtained from YTS 156 showed mutations at the 5′ and 3′ ends, but also showed two point mutations. One mutation in the light chain (residue 62, Kabat 72) is a Thr to Ser substitution and the other mutation found in CDR2 of the heavy chain (residue 58, Kabat 58) is a Glu to Asp substitution. Mb synthesis was made with the sequences obtained from the RT-PCR. For YTS 169, no sequence verification was performed. Both Mb and Cys-diabody (cDb) nucleic acid and amino acid sequences of YTS169, 2.43 and YTS 156 are listed below (Note: * is stop codon):

Anti-mCD8 YTS 169 minibody nucleic acid sequence (SEQ ID NO.: 38):

TCTAGAGCCGCCACCATGGAAACCGACACCCTGCTGCTGTGGGTGCTGC
TGCTCTGGGTCCCCGGAAGCACCGGTGAAGTGAAGCTGCAGGAAAGCGG
CGGAGGCCTGGTGCAGCCCGGCAGAAGCCTGAAGCTGAGCTGTGCCGCC
AGCGGCTTCAACTTCAACGACTACTGGATGGGCTGGGTCCGACAGGCTC
CTGGCAAGGGCCTGGAATGGATCGGCGAGATCAACAAGGACAGCAGCAC
CATCAACTACACCCCCAGCCTGAAGGACAAGTTCACCATCAGCAGAGAC
AACGCCCAGAACACCCTGTACCTGCAGATGAGCAAGCTGGGCAGCGAGG
ACACCGCCATCTACTACTGCGCCAGAGCCAGAGGCATGATGGTGCTGAT
CATCCCCCACTACTTCGACTACTGGGGCCAGGGCGTGATGGTCACCGTG
TCCAGCGGCTCTACCAGTGGCGGAGGATCTGGCGGAGGAAGCGGAGGCG
GCGGAAGCAGCGACATCGTGCTGACCCAGAGCCCCGCTATGGCCATGAG
CCCTGGCGAGAGAATCACAATCAGCTGCAGAGCCAGCGAGAGCGTGTCC
ACCAGAATGCACTGGTATCAGCAGAAGCCCGGCCAGCAGCCCAAGCTGC
TGATCTACGGCGCCAGCAACCTGGAATCCGGCGTGCCAGCCAGATTCAG
CGGCAGCGGCTCCGGCACCGACTTCACCCTGACCATCGACCCCGTGGAA
GCCAACGACACCGCCACCTATTTCTGCCAGCAGTCTTGGTACGACCCCT
GGACCTTCGGTGGAGGCACCAAGCTGGAACTGAAGCTCGAGCCCAGAGG
CCCCACCATCAAGCCCTGCCCTCCCTGCAAGTGCCCTGCCCCTAACCTG
CTGGGCGGACCTGGATCTGTGCGGGCTCCCCAGGTGTACGTGCTGCCCC
CACCCGAGGAAGAGATGACCAAGAAACAGGTGACACTGACCTGCATGGT
GACAGACTTCATGCCCGAGGATATCTACGTGGAATGGACCAACAACGGC
AAGACCGAGCTGAACTACAAGAACACCGAGCCCGTGCTGGACAGCGACG
GCAGCTACTTCATGTACAGCAAGCTGCGGGTGGAAAAGAAAAACTGGGT
GGAACGGAACAGCTACAGCTGCAGCGTGGTGCACGAGGGCCTGCACAAC
TACCACACCACCAAGAGCTTCAGCAGGACCCCCGGCAAAGCGGCCGCGG
CCGGCCATCTGCCTGAAACAGGGGCCGGCCACCACCACCATCACCATGC
TGGCCGCTGAGAATTC

Anti-mCD8 YTS 169 minibody amino acid sequence (SEQ ID NO.: 39):

SRAATMETDTLLLWVLLLWVPGSTGEVKLQESGGGLVQPGRSLKLSCAA
SGFNFNDYWMGWVRQAPGKGLEWIGEINKDSSTINYTPSLKDKFTISRD
NAQNTLYLQMSKLGSEDTAIYYCARARGMMVLIIPHYFDYWGQGVMVTV
SSGSTSGGGSGGGSGGGGSSDIVLTQSPAMAMSPGERITISCRASESVS
TRMHWYQQKPGQQPKLLIYGASNLESGVPARFSGSGSGTDFTLTIDPVE
ANDTATYFCQQSWYDPWTFGGGTKLELKLEPRGPTIKPCPPCKCPAPNL
LGGPGSVRAPQVYVLPPPEEEMTKKQVTLTCMVTDFMPEDIYVEWTNNG
KTELNYKNTEPVLDSDGSYFMYSKLRVEKKNWVERNSYSCSVVHEGLHN
YHTTKSFSRTPGKAAAAGHLPETGAGHHHHHHAAA*EF

Anti-mCD8 YTS 169 cDb nucleic acid sequence (SEQ ID NO.: 40):

TCTAGAGCCGCCACCATGGAAACCGACACCCTGCTGCTGTGGGTGCTGC
TGCTCTGGGTCCCCGGAAGCACCGGTGAAGTGAAGCTGCAGGAAAGCGG
CGGAGGCCTGGTGCAGCCCGGCAGAAGCCTGAAGCTGAGCTGTGCCGCC
AGCGGCTTCAACTTCAACGACTACTGGATGGGCTGGGTCCGACAGGCTC
CTGGCAAGGGCCTGGAATGGATCGGCGAGATCAACAAGGACAGCAGCAC
CATCAACTACACCCCCAGCCTGAAGGACAAGTTCACCATCAGCAGAGAC
AACGCCCAGAACACCCTGTACCTGCAGATGAGCAAGCTGGGCAGCGAGG
ACACCGCCATCTACTACTGCGCCAGAGCCAGAGGCATGATGGTGCTGAT
CATCCCCCACTACTTCGACTACTGGGGCCAGGGCGTGATGGTCACCGTG
TCCAGCGGCGGCGGAGGATCTGACATCGTGCTGACCCAGAGCCCCGCTA
TGGCCATGAGCCCTGGCGAGAGAATCACAATCAGCTGCAGAGCCAGCGA
GAGCGTGTCCACCAGAATGCACTGGTATCAGCAGAAGCCCGGCCAGCAG
CCCAAGCTGCTGATCTACGGCGCCAGCAACCTGGAATCCGGCGTGCCAG
CCAGATTCAGCGGCAGCGGCTCCGGCACCGACTTCACCCTGACCATCGA
CCCCGTGGAAGCCAACGACACCGCCACCTATTTCTGCCAGCAGTCTTGG
TACGACCCCTGGACCTTCGGTGGAGGCACCAAGCTGGAACTGAAGGCGG
CCGCAGGCTGCGGAGGCCACCACCACCATCACCATTGAGAATTC

Anti-mCD8 YTS 169 cDb amino acid sequence (SEQ ID NO.: 41):

SRAATMETDTLLLWVLLLWVPGSTGEVKLQESGGGLVQPGRSLKLSCAA
SGFNFNDYWMGWVRQAPGKGLEWIGEINKDSSTINYTPSLKDKFTISRD
NAQNTLYLQMSKLGSEDTAIYYCARARGMMVLIIPHYFDYWGQGVMVTV
SSGGGGSDIVLTQSPAMAMSPGERITISCRASESVSTRMHWYQQKPGQQ
PKLLIYGASNLESGVPARFSGSGSGTDFTLTIDPVEANDTATYFCQQSW
YDPWTFGGGTKLELKAAAGCGGHHHHHH*EF

Anti-mCD8 2.43 Mb nucleic acid sequence (SEQ ID NO.: 42):

TCTAGAGCCGCCACCATGGAAACCGACACCCTGCTGCTGTGGGTGCTGC
TGCTCTGGGTGCCCGGCAGCACCGGTGAAGTGCAGCTGGTGGAAAGCGG
CGGAGGCCTGGTGCAGCCCGGCAGAAGCCTGAAGCTGAGCTGTGCCGCC
AGCGGCTTCACCTTCAGCAACTACTACATGGCTTGGGTGCGCCAGGCCC
CCACCAAGGGACTGGAATGGGTGGCCTACATCAACACCGGCGGAGGCAC
CACCTACTACAGAGACAGCGTGAAGGGCAGATTCACCATCAGCAGGGAC
GACGCCAAGAGCACCCTGTACCTGCAGATGGACAGCCTGAGAAGCGAGG
ACACCGCTACCTACTACTGCACCACCGCCATCGGCTACTACTTCGACTA
CTGGGGCCAGGGCGTGATGGTGACAGTGTCCAGCGGCAGCACCTCTGGC
GGCGGATCTGGCGGAGGAAGCGGAGGCGGCGGAAGCAGCGACATCCAGC
TGACACAGAGCCCCGCCAGCCTGAGCGCCTCTCTGGGCGAGACAGTGTC
TATCGAGTGCCTGGCCAGCGAGGACATCTACAGCTACCTGGCCTGGTAT
CAGCAGAAGCCCGGCAAGAGCCCCCAGGTGCTGATCTACGCCGCCAACA
GACTGCAGGACGGCGTGCCCAGCAGATTCAGCGGCTCTGGCAGCGGCAC
CCAGTACAGCCTGAAGATCAGCGGCATGCAGCCCGAGGACGAGGGCGAC
TACTTCTGTCTGCAGGGCAGCAAGTTCCCCTACACCTTCGGCGCTGGCA
CCAAGCTGGAACTGAAGCTCGAGCCCAGAGGCCCCACCATCAAGCCCTG
CCCTCCCTGCAAGTGCCCTGCCCCTAACCTGCTGGGCGGACCTGGATCT
GTGCGGGCTCCCCAGGTGTACGTGCTGCCCCCACCCGAGGAAGAGATGA
CCAAGAAACAGGTGACACTGACCTGCATGGTGACAGACTTCATGCCCGA
GGATATCTACGTGGAATGGACCAACAACGGCAAGACCGAGCTGAACTAC
AAGAACACCGAGCCCGTGCTGGACAGCGACGGCAGCTACTTCATGTACA
GCAAGCTGCGGGTGGAAAAGAAAAACTGGGTGGAACGGAACAGCTACAG
CTGCAGCGTGGTGCACGAGGGCCTGCACAACTACCACACCACCAAGAGC
TTCAGCAGGACCCCCGGCAAAGCGGCCGCGGCCGGCCATCTGCCTGAAA
CAGGGGCCGGCCACCACCACCATCACCATGCGGCCGCTTGAGAATTC

Anti-mCD8 2.43 Mb amino acid sequence (SEQ ID NO.: 43):

SRAATMETDTLLLWVLLLWVPGSTGEVQLVESGGGLVQPGRSLKLSCAA
SGFTFSNYYMAWVRQAPTKGLEWVAYINTGGGTTYYRDSVKGRFTISRD
DAKSTLYLQMDSLRSEDTATYYCTTAIGYYFDYWGQGVMVTVSSGSTSG
GGSGGGSGGGGSSDIQLTQSPASLSASLGETVSIECLASEDIYSYLAWY
QQKPGKSPQVLIYAANRLQDGVPSRFSGSGSGTQYSLKISGMQPEDEGD
YFCLQGSKFPYTFGAGTKLELKLEPRGPTIKPCPPCKCPAPNLLGGPGS
VRAPQVYVLPPPEEEMTKKQVTLTCMVTDFMPEDIYVEWTNNGKTELNY
KNTEPVLDSDGSYFMYSKLRVEKKNWVERNSYSCSVVHEGLHNYHTTKS
FSRTPGKAAAAGHLPETGAGHHHHHHAAA*EF

Anti-mCD8 2.43 cDb nucleic acid sequence (SEQ ID NO.: 44):

TCTAGAGCCGCCACCATGGAAACCGACACCCTGCTGCTGTGGGTGCTGC
TGCTCTGGGTGCCCGGCAGCACCGGTGAAGTGCAGCTGGTGGAAAGCGG
CGGAGGCCTGGTGCAGCCCGGCAGAAGCCTGAAGCTGAGCTGTGCCGCC
AGCGGCTTCACCTTCAGCAACTACTACATGGCTTGGGTGCGCCAGGCCC
CCACCAAGGGACTGGAATGGGTGGCCTACATCAACACCGGCGGAGGCAC
CACCTACTACAGAGACAGCGTGAAGGGCAGATTCACCATCAGCAGGGAC
GACGCCAAGAGCACCCTGTACCTGCAGATGGACAGCCTGAGAAGCGAGG
ACACCGCTACCTACTACTGCACCACCGCCATCGGCTACTACTTCGACTA
CTGGGGCCAGGGCGTGATGGTGACAGTGTCCAGCGGCGGCGGAGGAAGC
GACATCCAGCTGACACAGAGCCCCGCCAGCCTGAGCGCCTCTCTGGGCG
AGACAGTGTCTATCGAGTGCCTGGCCAGCGAGGACATCTACAGCTACCT
GGCCTGGTATCAGCAGAAGCCCGGCAAGAGCCCCCAGGTGCTGATCTAC
GCCGCCAACAGACTGCAGGACGGCGTGCCCAGCAGATTCAGCGGCTCTG
GCAGCGGCACCCAGTACAGCCTGAAGATCAGCGGCATGCAGCCCGAGGA
CGAGGGCGACTACTTCTGTCTGCAGGGCAGCAAGTTCCCCTACACCTTC
GGCGCTGGCACCAAGCTGGAACTGAAGGCGGCCGCAGGCTGCGGAGGCC
ACCACCACCATCACCATTGAGAATTC

Anti-mCD8 2.43 cDb amino acid sequence (SEQ ID NO.: 45):

SRAATMETDTLLLWVLLLWVPGSTGEVQLVESGGGLVQPGRSLKLSCAA
SGFTFSNYYMAWVRQAPTKGLEWVAYINTGGGTTYYRDSVKGRFTISRD
DAKSTLYLQMDSLRSEDTATYYCTTAIGYYFDYWGQGVMVTVSSGGGGS
DIQLTQSPASLSASLGETVSIECLASEDIYSYLAWYQQKPGKSPQVLIY
AANRLQDGVPSRFSGSGSGTQYSLKISGMQPEDEGDYFCLQGSKFPYTF
GAGTKLELKAAAGCGGHHHHHH*EF

Anti-mCD8 YTS 156 Mb nucleic acid sequence (SEQ ID NO.: 46):

TCTAGAGCCGCCACCATGGAAACCGACACCCTGCTGCTGTGGGTGCTGC
TGCTCTGGGTCCCCGGAAGCACCGGTGAAGTGAAGCTGCAGGAAAGCGG
CCCCAGCCTGGTGCAGCCTAGCCAGACCCTGAGCCTGACCTGCAGCGTG
TCCGGCTTCAGCCTGATCAGCGACAGCGTGCACTGGGTCCGACAGCCTC
CCGGCAAGGGCCTGGAATGGATGGGCGGCATCTGGGCCGACGGCTCCAC
CGACTACAACAGCGCCCTGAAGTCCAGACTGAGCATCAGCAGAGACACC
AGCAAGAGCCAGGGCTTCCTGAAGATGAACAGCCTGCAGACCGACGACA
CCGCCATCTATTTCTGCACCAGCAACCGCGAGAGCTACTACTTCGACTA
CTGGGGCCAGGGCGTGATGGTCACCGTGTCCAGCGGCTCTACCAGCGGC
GGAGGCTCTGGCGGAGGATCTGGTGGCGGCGGAAGCAGCGACATCCAGA
TGACCCAGAGCCCTGCCAGCCTGAGCGCCAGCCTGGGCGACAAAGTGAC
CATCACCTGTCAGGCCAGCCAGAACATCGACAAGTATATCGCCTGGTAT
CAGCAGAAGCCTGGCAAGGCCCCCAGACAGCTGATCCACTACACCAGCA
CACTGGTGTCCGGCACCCCCAGCAGATTCAGCGGCAGCGGCTCCGGCAG
AGACTACAGCTTCAGCATCAGCTCCGTGGAAAGCGAGGATATCGCCAGC
TACTACTGCCTGCAGTACGACACCCTGTACACCTTCGGCGCTGGCACCA
AGCTGGAACTGAAGCTCGAGCCCAGAGGCCCCACCATCAAGCCCTGCCC
TCCCTGCAAGTGCCCTGCCCCTAACCTGCTGGGCGGACCTGGATCTGTG
CGGGCTCCCCAGGTGTACGTGCTGCCCCCACCCGAGGAAGAGATGACCA
AGAAACAGGTGACACTGACCTGCATGGTGACAGACTTCATGCCCGAGGA
TATCTACGTGGAATGGACCAACAACGGCAAGACCGAGCTGAACTACAAG
AACACCGAGCCCGTGCTGGACAGCGACGGCAGCTACTTCATGTACAGCA
AGCTGCGGGTGGAAAAGAAAAACTGGGTGGAACGGAACAGCTACAGCTG
CAGCGTGGTGCACGAGGGCCTGCACAACTACCACACCACCAAGAGCTTC
AGCAGGACCCCCGGCAAAGCGGCCGCGGCCGGCCATCTGCCTGAAACAG
GGGCCGGCCACCACCACCATCACCATGCGGCCGCTTGAGAATTC

Anti-mCD8 YTS 156 Mb amino acid sequence (SEQ ID NO.: 47):

SRAATMETDTLLLWVLLLWVPGSTGEVKLQESGPSLVQPSQTLSLTCSV
SGFSLISDSVHWVRQPPGKGLEWMGGIWADGSTDYNSALKSRLSISRDT
SKSQGFLKMNSLQTDDTAIYFCTSNRESYYFDYWGQGVMVTVSSGSTSG
GGSGGGSGGGGSSDIQMTQSPASLSASLGDKVTITCQASQNIDKYIAWY
QQKPGKAPRQLIHYTSTLVSGTPSRFSGSGSGRDYSFSISSVESEDIAS
YYCLQYDTLYTFGAGTKLELKLEPRGPTIKPCPPCKCPAPNLLGGPGSV
RAPQVYVLPPPEEEMTKKQVTLTCMVTDFMPEDIYVEWTNNGKTELNYK
NTEPVLDSDGSYFMYSKLRVEKKNWVERNSYSCSVVHEGLHNYHTTKSF
SRTPGKAAAAGHLPETGAGHHHHHHAAA*EF

Anti-mCD8 YTS 156 cDb nucleic acid sequence (SEQ ID NO.: 48):

TCTAGAGCCGCCACCATGGAAACCGACACCCTGCTGCTGTGGGTGCTGC
TGCTCTGGGTCCCCGGAAGCACCGGTGAAGTGAAGCTGCAGGAAAGCGG
CCCCAGCCTGGTGCAGCCTAGCCAGACCCTGAGCCTGACCTGCAGCGTG
TCCGGCTTCAGCCTGATCAGCGACAGCGTGCACTGGGTCCGACAGCCTC
CCGGCAAGGGCCTGGAATGGATGGGCGGCATCTGGGCCGACGGCTCCAC
CGACTACAACAGCGCCCTGAAGTCCAGACTGAGCATCAGCAGAGACACC
AGCAAGAGCCAGGGCTTCCTGAAGATGAACAGCCTGCAGACCGACGACA
CCGCCATCTATTTCTGCACCAGCAACCGCGAGAGCTACTACTTCGACTA
CTGGGGCCAGGGCGTGATGGTCACCGTGTCCAGCGGCGGCGGAGGCTCT
GACATCCAGATGACCCAGAGCCCTGCCAGCCTGAGCGCCAGCCTGGGCG
ACAAAGTGACCATCACCTGTCAGGCCAGCCAGAACATCGACAAGTATAT
CGCCTGGTATCAGCAGAAGCCTGGCAAGGCCCCCAGACAGCTGATCCAC
TACACCAGCACACTGGTGTCCGGCACCCCCAGCAGATTCAGCGGCAGCG
GCTCCGGCAGAGACTACAGCTTCAGCATCAGCTCCGTGGAAAGCGAGGA
TATCGCCAGCTACTACTGCCTGCAGTACGACACCCTGTACACCTTCGGC
GCTGGCACCAAGCTGGAACTGAAGGCGGCCGCAGGCTGCGGAGGCCACC
ACCACCATCACCATTGAGAATTC

Anti-mCD8 YTS 156 cDb amino acid sequence (SEQ ID NO.: 49):

SRAATMETDTLLLWVLLLWVPGSTGEVKLQESGPSLVQPSQTLSLTCSV
SGFSLISDSVHWVRQPPGKGLEWMGGIWADGSTDYNSALKSRLSISRDT
SKSQGFLKMNSLQTDDTAIYFCTSNRESYYFDYWGQGVMVTVSSGGGGS
DIQMTQSPASLSASLGDKVTITCQASQNIDKYIAWYQQKPGKAPRQLIH
YTSTLVSGTPSRFSGSGSGRDYSFSISSVESEDIASYYCLQYDTLYTFG
AGTKLELKAAAGCGGHHHHHH*EF

Production and Characterization of Minibody Fragments.

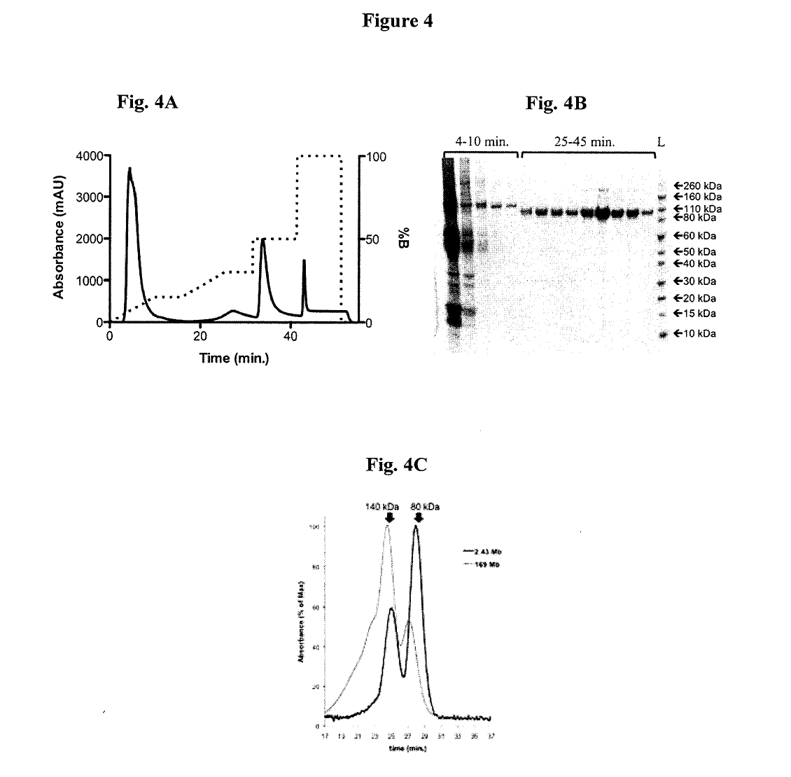

Minibody purification was performed using NiNTA columns and imidazole elution (FIG. 4A). SDS-PAGE showed the minibody eluted between 25-45 minutes (FIG. 4B). The yields of the 2.43 and YTS169 minibodies were 6.6 and 8.9 mg/L, respectively. Purified protein was then run on Superdex200 size exclusion chromatography for elution profiles and compared to reference standards to confirm assembly, purity and dimerization (FIG. 4C). The 2.43 and YTS169 minibodies are purified as 81 or 23% 80 kDa dimers, respectively, as calculated by the area under the curve, with the remaining 19 and 77% eluting as higher molecular weight multimers.

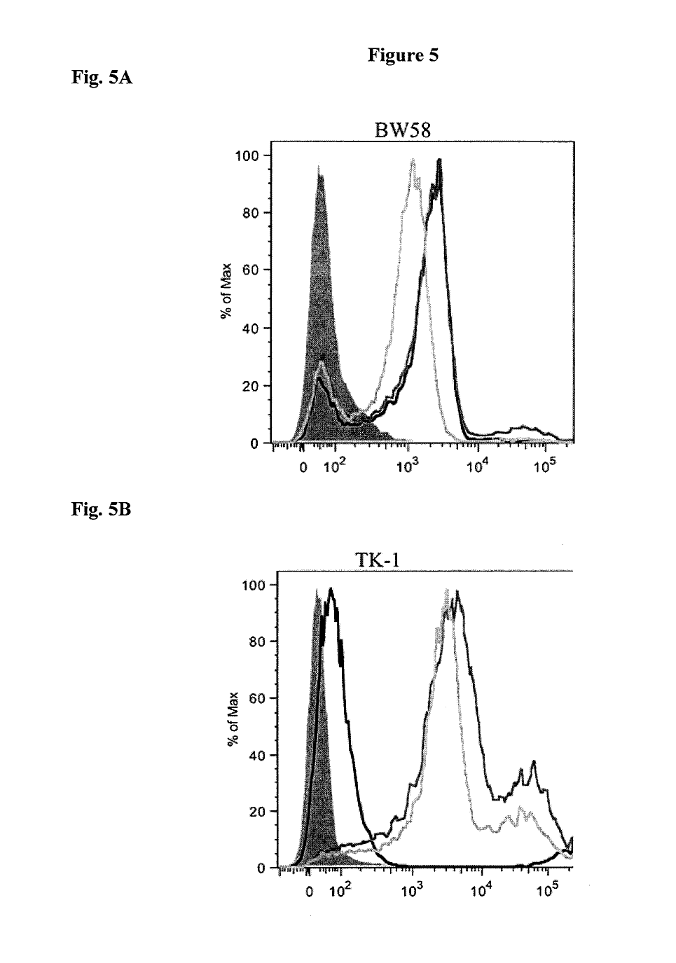

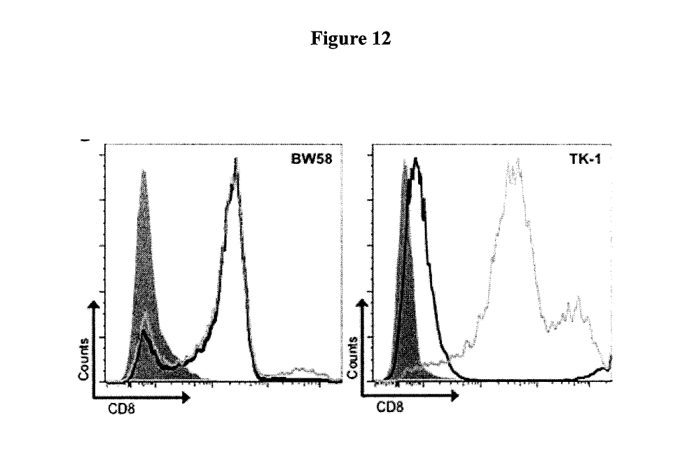

Epitope binding was assessed by flow cytometry. Initial flow cytometry confirmed epitope specificity of the minibody fragments. The murine CD8⁺ T cell lymphoma lines BW58 (Lyt2.2⁺) and TK-1 (Lyt2.1⁺) were stained with either 2.43 or YTS169 Mb, followed by anti-mouse IgG2a-PE (FIG. 5A-5B and FIG. 12).

For example, the sCD8αβ antigen was first tested for binding to the three produced minibodies by size exclusion chromotography. Briefly, equimolar amounts of soluble antigen and the minibody in question were incubated for five minutes in PBS before SE200 analysis. All Mb peaks eluted about 2.8 to 3 min. earlier in the presence of sCD8αβ. Additionally, some minibody aggregation detected by size exclusion also eluted 2.5 to 2.8 minutes earlier.

Initially, the 2.43 minibody was conjugated to SCN-NOTA for ⁶⁴Cu radiolabeling and subsequent PET imaging and biodistribution studies. The 2.43 Mb showed affinity for the soluble antigen after SCN-NOTA conjugation, as seen by SE200 analysis. ⁶⁴Cu radiolabeling efficiency was generally >80% and the radiochemical purity was always >98% after spin column purification. The immunoreactive fraction of ⁶⁴Cu-NOTA 2.43 minibody varied between 65-75%. The specific activity was between 8 to 10 mCi/mg and mice were injected with about 70-80 mCi intravenously. After 4 hours, a PET image was acquired and the mice were sacrificed for biodistribution analysis. Due to the specificity for Lyt2.2, wild type C57BL/6 (Lyt2.2⁺) and C3H (Lyt2.1⁺) mice were imaged with ⁶⁴Cu-NOTA-2.43 Mb. PET images showed high uptake in the spleen, lymph nodes, bone marrow and liver of the antigen positive B/6 mice (FIG. 8). Interestingly, the thymus did not show high uptake. The average % ID/g organs are listed in Table 1. When injected into C3H mice, the ⁶⁴Cu-NOTA-2.43 Mb showed similar uptake in the liver, and significant but reduced uptake the spleen, lymph nodes, and bone marrow when compared to the B/6 mice. The average % ID/g blood after only four hours, 0.81 and 1.34 for B/6 and C3H mice respectively, is low for both mice strains.

TABLE 1
Biodistribution of 64Cu-NOTA 2.43 Mb at 4 h post-injection in B/6,
C3H and NSG SCID mice represented as % ID/g (mean ± SD; n = 3-4).

| Organ | Wild type B/6 | Wild type C3H | NSG SCID |
|---|---|---|---|
| Blood | 0.81 ± 0.06 | 1.34 ± 0.10 | 0.89 ± 0.13 |
| Axillary LNs | 33.13 ± 5.33 | 2.68 ± 0.71 | N/A |
| Spleen | 69.24 ± 1.96 | 15.43 ± 2.35 | 12.9 ± 3.93 |
| Thymus | 0.58 ± 0.02 | 0.46 ± 0.05 | 2.21 ± 0.89 |
| Bone | 6.14 ± 0.43 | 4.01 ± 0.34 | 3.61 ± 0.49 |
| Stomach | 0.90 ± 0.38 | 1.11 ± 0.11 | 0.43 ± 0.11 |
| Intestines | 3.41 ± 0.29 | 3.22 ± 0.17 | 1.06 ± 0.04 |
| Liver | 47.63 ± 1.94 | 47.28 ± 1.59 | 37.63 ± 0.80 |
| Kidneys | 5.71 ± 1.03 | 5.85 ± 0.72 | 3.77 ± 0.26 |
| Heart | 1.40 ± 0.07 | 2.29 ± 0.09 | 1.29 ± 0.15 |
| Lungs | 3.73 ± 1.82 | 2.27 ± 0.61 | 1.25 ± 0.11 |
| Muscle | 0.14 ± 0.02 | 0.24 ± 0.01 | 0.15 ± 0.03 |

To further analyze the uptake in immunological organs of C3H mice possibly due to low affinity of the 2.43 Mb to Lyt2.1, immunodeficient NSG mice were injected with the ⁶⁴Cu-NOTA-2.43 Mb because they have no mature T cells and NK cells as they lack the IL2 receptor gamma chain. Uptake in these mice were very similar to that of the ⁶⁴Cu-NOTA-2.43 minibody in C3H mice (FIG. 8 and Table 1). Axillary lymph nodes were not harvested from NSG mice due to difficulties in detection/isolation. Although PET images do not show high uptake in the spleen of the NSG mice, the % ID/g is comparable to that of the C3H antigen negative mice because of the small size of the NSG spleens (NSG average spleen: 32.4 mg, C3H average spleen weight: 83.2 mg)

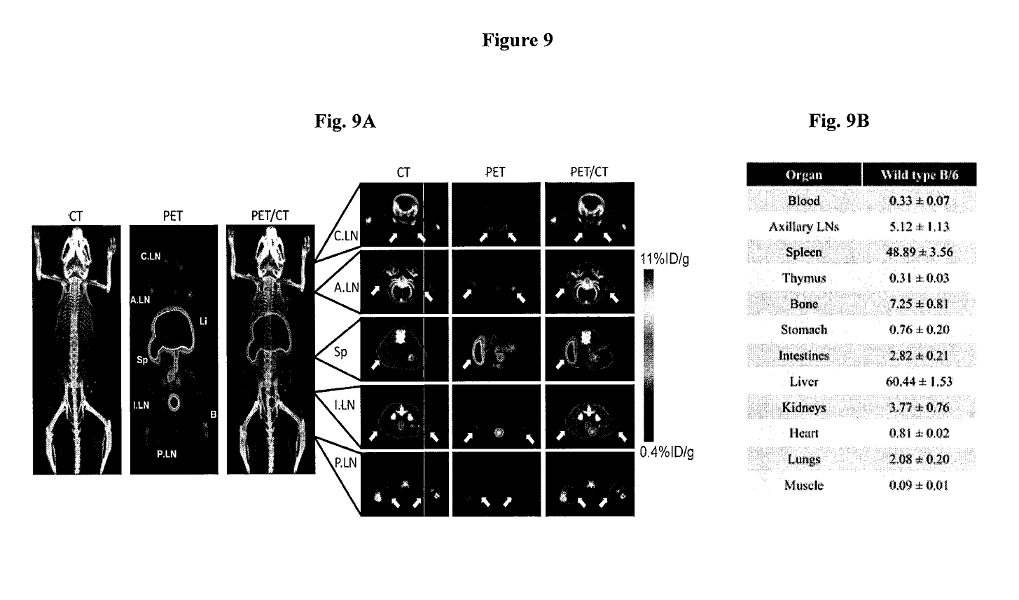

For the YTS 169 Mb, the radiolabeling, specific activity and immunoreactive fraction was very similar to the ⁶⁴Cu-NOTA 2.43 minibody. The PET imaging and biodistributions in wild type B/6 mice using the ⁶⁴Cu-NOTA YTS 169 Mb were similar to those of ⁶⁴Cu-NOTA 2.43 Mb in B/6 mice (FIG. 9A-9B). It should be noted, however, that the % ID/g values are not consistent for every experiment but they are very reproducible among groups of mice on the same day in the same experiment. Furthermore, very high uptake in the lymph nodes is detectable with PET imaging when the intravenous tail injection is poor.

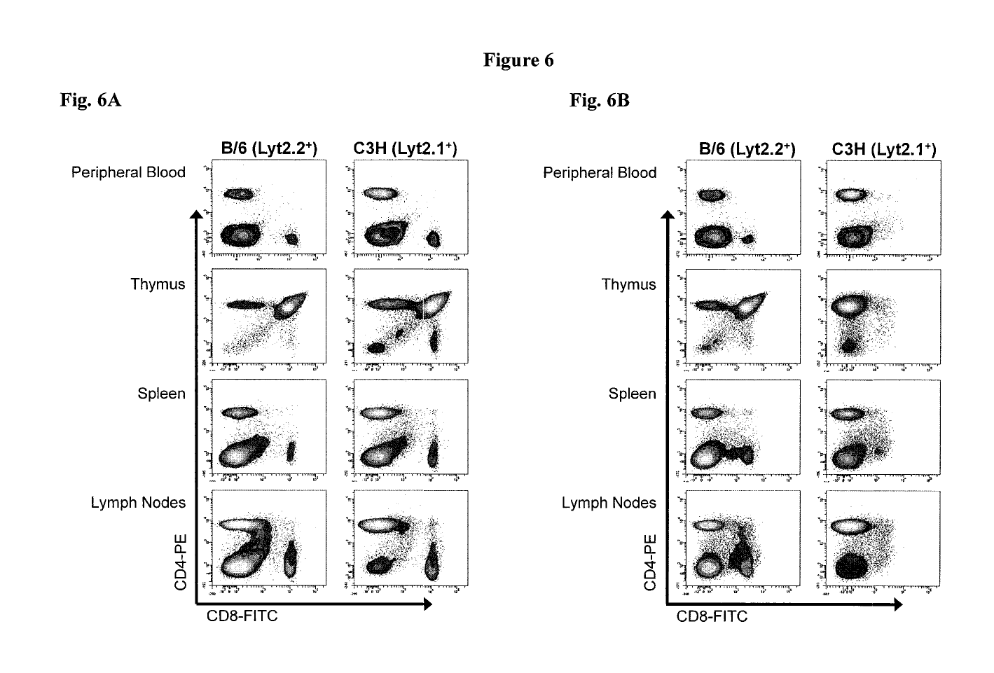

To further demonstrate the epitope specificity of the 2.43 Mb construct, the 2.43 minibody was conjugated to FITC to a 1.4:1 ratio of fluorescein:Mb. Single cell suspensions from the peripheral blood, thymus, spleen and lymph nodes of B/6 (Lyt2.2⁺) or C3H (Lyt2.1⁺) mice were stained with either the FITC-2.43 minibody or a commercial FITC-anti-CD8 antibody and anti-CD4 (FIG. 6). The 2.43 Mb shows comparable binding to cells isolated from antigen-positive Lyt2.2 B/6 mice and does not bind CD8 in primary cells from various organs of the antigen-negative Lyt2.1 C3H mice.

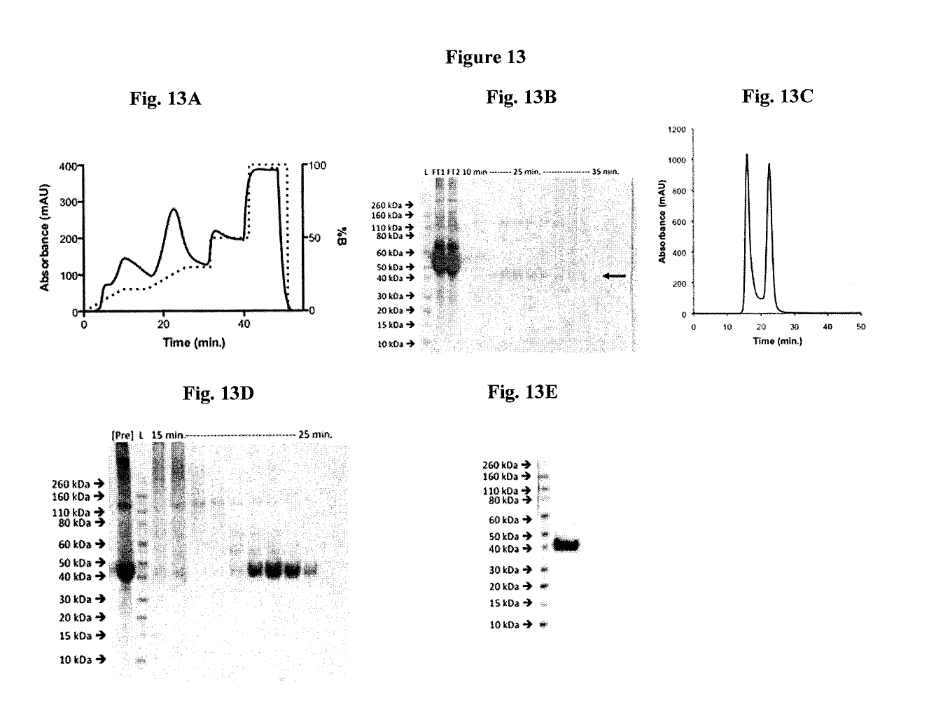

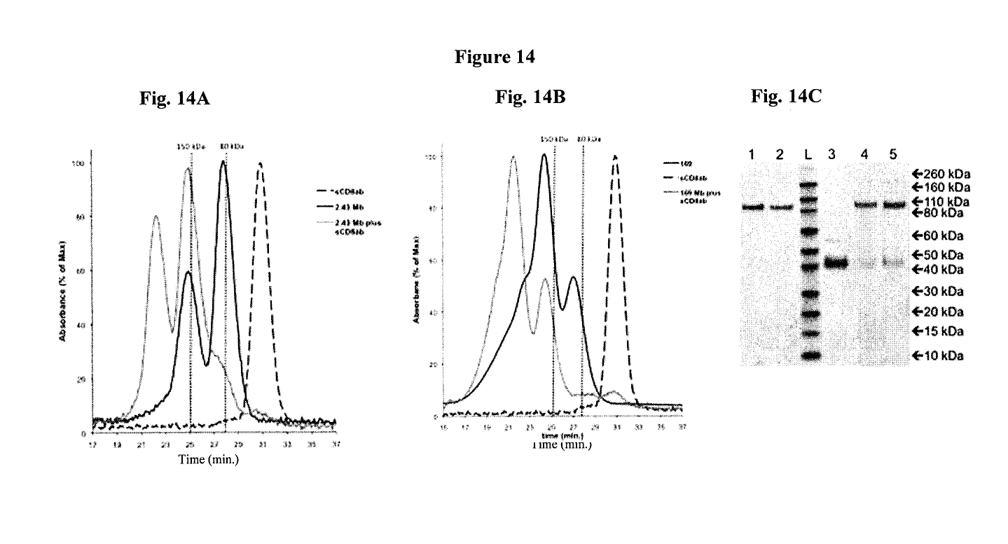

To determine the affinity of both the 2.43 and YTS169 Mb, a soluble CD8αβ heterodimer fusion protein was constructed by removing the transmembrane domains of both CD8α and CD8β and fusing them with a 29 amino acid alpha helical linker. The soluble antigen was purified using NiNTA affinity chromatography followed by size exclusion chromatography (FIG. 13). Solution-phase binding of the 2.43 and YTS169 Mbs to sCD8αβ antigen was first confirmed by SEC. Briefly, equimolar amounts of soluble antigen and the minibody in question were incubated for five minutes in PBS before SE200 analysis. All minibody peaks eluted 2.8-3 min. earlier in the presence of sCD8αβ confirming binding of minibody and antigen complexes of a larger size. Additionally, the minibody multimer detected by size exclusion eluted 2.5-2.8 minutes earlier (FIG. 14).

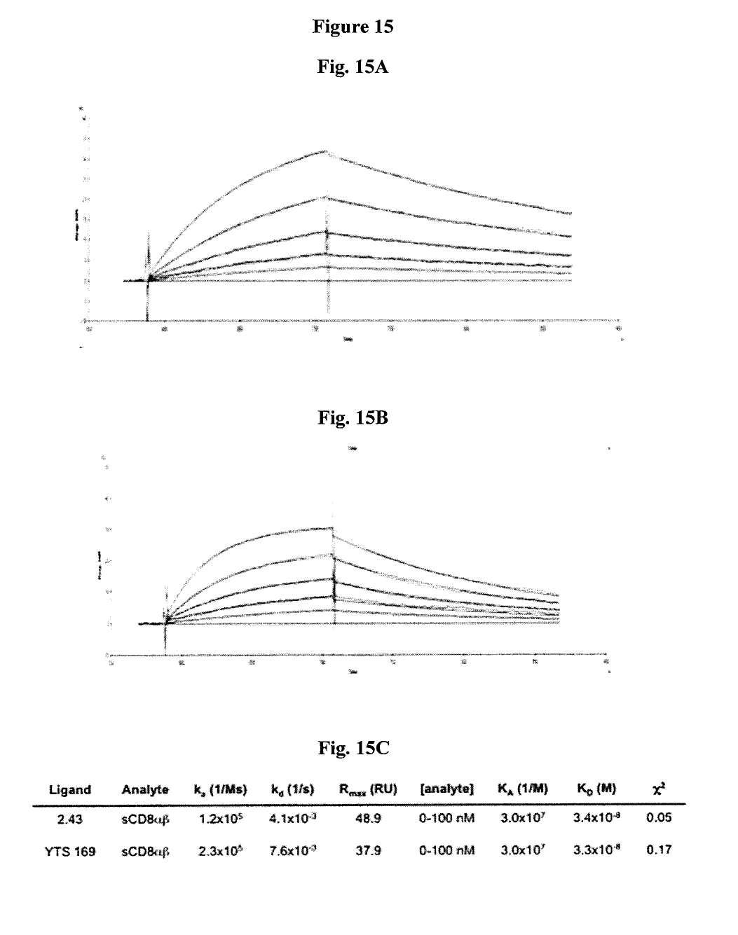

SPR kinetic analysis was performed using the Biacore 3000 with immobilized minibody and soluble monomeric sCD8αβ. The equilibrium constant (K_{D}) for 2.43 and YTS169 were 34 and 33 nM, respectively (FIG. 15).

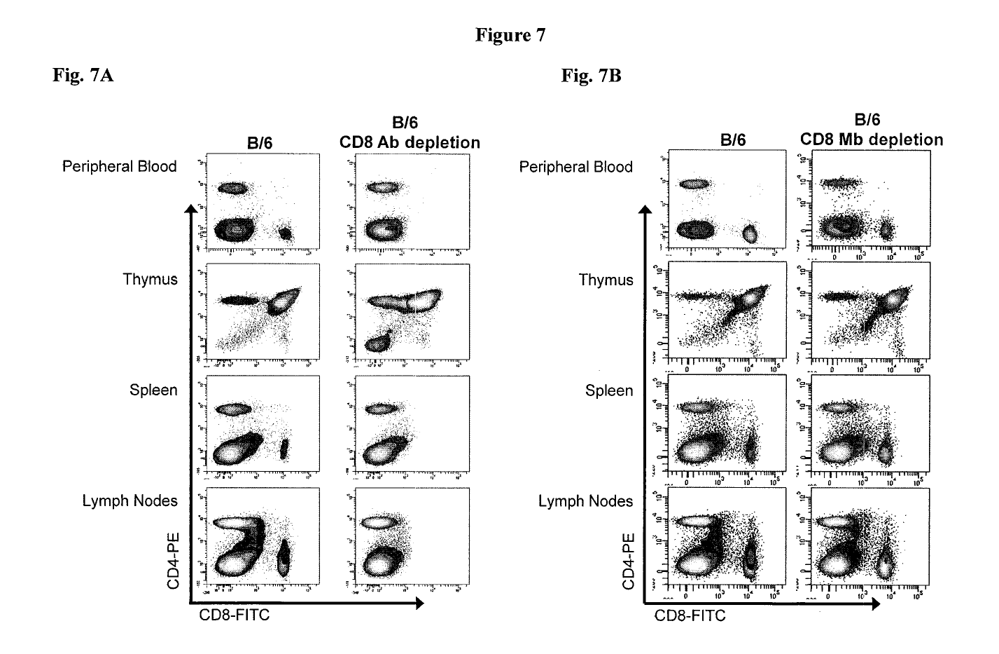

In vivo depletion studies were conducted. Single cell suspensions from the spleen, peripheral blood, thymus and lymph nodes of wt B/6 mice, B/6 mice treated with a CD8 depleting antibody, or B/6 mice treated with the 2.43 minibody were analyzed by flow cytometry for effective CD8 depletion (FIG. 7). Mice treated with the depleting Ab showed >95% CD8 depletion while mice treated with the 2.43 minibody did not show a reduction in CD8 expressing cells.

NOTA conjugation and radiolabeling were performed. Following conjugation of both 2.43 and YTS169 minibody to SCN-NOTA and ⁶⁴Cu radiolabeling, efficient ⁶⁴Cu radiolabeling was mostly >80% and the radiochemical purity was >98% after spin column purification. The immunoreactive fraction of the ⁶⁴Cu-NOTA Mbs varied between 65-75%. The specific activity was between 295-370 MBq/mg (8-10 mCi/mg) and mice were injected with 2.6-2.9 MBq (70-80 μCi) intravenously.

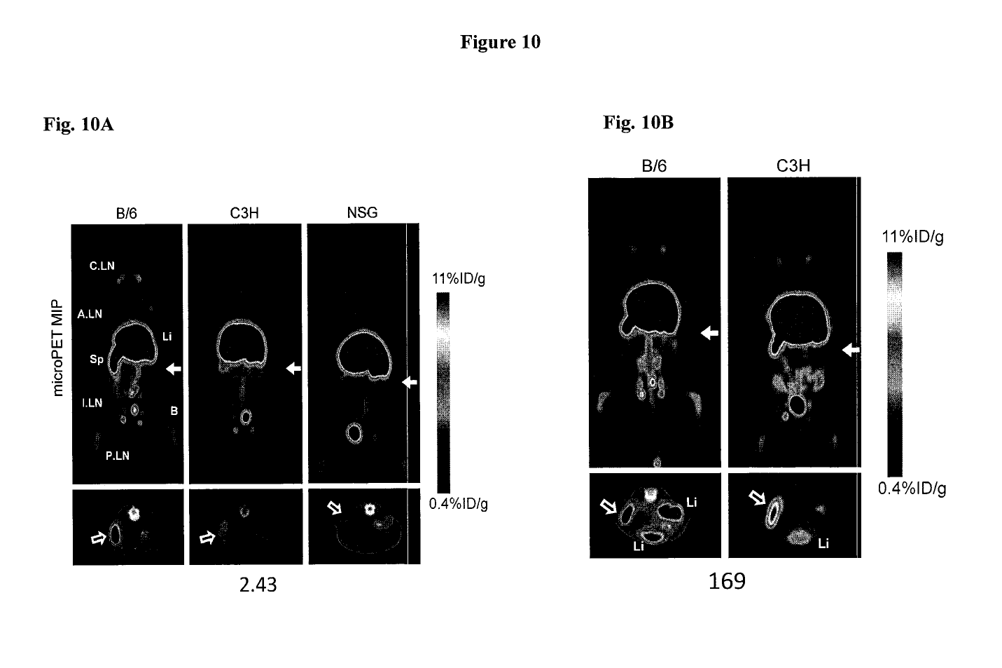

ImmunoPET and ex vivo biodistribution were performed. Due to the specificity for Lyt2.2, wild type B/6 (Lyt2.2⁺) mice were initially imaged with ⁶⁴Cu-NOTA-2.43 minibody (FIG. 9). High contrast immunoPET images showed high % ID/g uptake in the spleen, lymph nodes and liver of the antigen-positive B/6 mice. The average % ID/g organs are listed in Table 2. When injected into antigen-negative Lyt2.1 C3H mice, the ⁶⁴Cu-NOTA-2.43 Mb showed similar % ID/g uptake in the liver and reduced uptake the spleen and lymph nodes when compared to the B/6 mice (FIG. 10A and Table 2). The average % ID/g blood after only four hours in B/6 and C3H mice was 0.81 and 1.34% ID/g, respectively.

TABLE 2
Ex vivo biodistribution analysis of the ⁶⁴Cu-NOTA-2.43 minibody four hours post-
injection in Lyt2.2⁺ B/6 mice, Lyt2.1⁺ C3H mice, NSG scid mice, antigen-blocked B/6 mice
and antigen-depleted B/6 mice.

| | % ID/g | | | | |
|---|---|---|---|---|---|
| | Wild Type | Wild Type | | | B/6 + |
| | B/6 | C3H | NSG | B/6 + Block | Depletion |
| Organ | (n = 6) | (n = 3) | (n = 3) | (n = 3) | (n = 3) |
| Blood | 0.90 ± 0.14 | 1.34 ± 0.10 | 0.89 ± 0.13 | 2.10 ± 0.31 | 1.90 ± 0.10 |
| Axillary | 27.58 ± 7.94 | 2.68 ± 0.71 | N/A | 5.00 ± 1.26 | 4.49 ± 2.68 |
| LNs | | | | | |
| Spleen | 75.00 ± 8.46 | 15.43 ± 2.35 | 12.90 ± 3.93 | 17.81 ± 1.94 | 14.82 ± 1.03 |
| Stomach | 1.08 ± 0.42 | 1.11 ± 0.11 | 0.43 ± 0.11 | 0.98 ± 0.09 | 1.59 ± 0.92 |
| Intestines | 3.80 ± 0.58 | 3.22 ± 0.17 | 1.06 ± 0.04 | 4.35 ± 0.47 | 3.43 ± 0.73 |
| Liver | 57.34 ± 11.39 | 47.28 ± 1.59 | 37.63 ± 0.90 | 70.92 ± 1.18 | 59.41 ± 6.47 |
| Kidneys | 5.57 ± 0.72 | 5.85 ± 0.72 | 3.77 ± 0.26 | 7.02 ± 0.31 | 6.44 ± 0.50 |
| Thymus | 0.89 ± 0.63 | 0.46 ± 0.05 | 2.21 ± 0.89 | 1.79 ± 0.42 | 1.12 ± 0.46 |
| Heart | 1.57 ± 0.22 | 2.29 ± 0.09 | 1.29 ± 0.15 | 3.08 ± 0.36 | 2.71 ± 0.19 |
| Lungs | 3.27 ± 1.27 | 2.27 ± 0.61 | 1.25 ± 0.11 | 3.07 ± 0.05 | 2.42 ± 0.48 |
| Muscle | 0.16 ± 0.03 | 0.24 ± 0.01 | 0.15 ± 0.03 | 0.42 ± 0.06 | 0.30 ± 0.06 |
| Bone | 8.25 ± 2.47 | 4.01 ± 0.34 | 3.61 ± 0.49 | 9.25 ± 0.70 | 8.95 ± 1.49 |
| Tail | 4.40 ± 2.82 | 6.02 ± 4.64 | 4.72 ± 4.94 | 6.75 ± 2.00 | 17.24 ± 4.32 |
| Carcass | 0.91 ± 0.15 | 0.83 ± 0.04 | 0.53 ± 0.03 | 1.40 ± 0.12 | 1.35 ± 0.13 |

To examine the unspecific uptake of ⁶⁴Cu-NOTA-2.43 minibody in the livers of antigen-negative C3H mice, the ⁶⁴Cu-NOTA-2.43 minibody was injected into immunodeficient NSG mice that lack mature T cells, B cells and natural killer cells. ImmunoPET images and ex vivo biodistribution in NSG mice were very similar to that of the ⁶⁴Cu-NOTA-2.43 minibody in antigen-negative C3H mice, confirming the high liver uptake as unspecific hepatic clearance of the radiolabeled Mb (FIG. 10A and Table 2).

For the YTS169 minibody, the radiolabeling, specific activity and immunoreactive fraction were similar to the ⁶⁴Cu-NOTA-2.43 minibody. The ImmunoPET imaging and ex vivo biodistributions in wild type B/6 mice using the ⁶⁴Cu-NOTA-YTS169 minibody were similar to those of ⁶⁴Cu-NOTA-2.43 minibody in B/6 mice (FIG. 10B and Table 3). Interestingly, the % ID/g in the spleen and liver of the ⁶⁴Cu-NOTA-YTS169 Mb in C3H mice is much lower (FIG. 5B and Table 2).

TABLE 3
Ex vivo biodistribution analysis of ⁶⁴Cu-NOTA-YTS169 minibody four hours post-
injection in Lyt2.2⁺ B/6 mice and Lyt2.1⁺ C3H mice.

| | % ID/g | | % ID | |
|---|---|---|---|---|
| | Wild type B/6 | Wild Type C3H | Wild type B/6 | Wild Type C3H |
| Organ | (n = 3) | (n = 3) | (n = 3) | (n = 3) |
| Blood | 0.33 ± 0.07 | 0.25 ± 0.01 | N/A | N/A |
| Axillary LNs | 5.12 ± 1.13 | 5.84 ± 0.66 | 0.07 ± 0.02 | 0.16 ± 0.10 |
| Spleen | 48.89 ± 3.56 | 25.63 ± 1.67 | 4.54 ± 0.16 | 3.96 ± 0.11 |
| Stomach | 0.76 ± 0.20 | 0.40 ± 0.15 | 0.43 ± 0.06 | 0.29 ± 0.04 |
| Intestines | 2.82 ± 0.21 | 2.26 ± 0.34 | 5.56 ± 0/75 | 5.11 ± 0.56 |
| Liver | 60.44 ± 1.53 | 43.08 ± 2.19 | 67.06 ± 1.73 | 65.04 ± 8.44 |
| Kidneys | 3.77 ± 0.76 | 2.55 ± 0.20 | 1.30 ± 0.36 | 1.17 ± 0.03 |
| Thymus | 0.31 ± 0.03 | 0.33 ± 0.09 | 0.02 ± 0.01 | 0.02 ± 0.01 |
| Heart | 0.81 ± 0.02 | 0.90 ± 0.07 | 0.12 ± 0.01 | 0.14 ± 0.01 |
| Lungs | 2.08 ± 0.20 | 1.95 ± 0.11 | 0.37 ± 0.05 | 0.44 ± 0.03 |
| Muscle | 0.09 ± 0.01 | 0.06 ± 0.01 | 0.01 ± 0.00 | 0.01 ± 0.00 |
| Bone | 7.25 ± 0.81 | 3.06 ± 0.28 | 0.64 ± 0.05 | 0.38 ± 0.01 |
| Tail | 6.58 ± 3.24 | 8.88 ± 9.49 | 4.47 ± 2.27 | 8.87 ± 9.95 |
| Carcass | 0.69 ± 0.05 | 0.53 ± 0.01 | 14.46 ± 1.01 | 15.05 ± 0.09 |

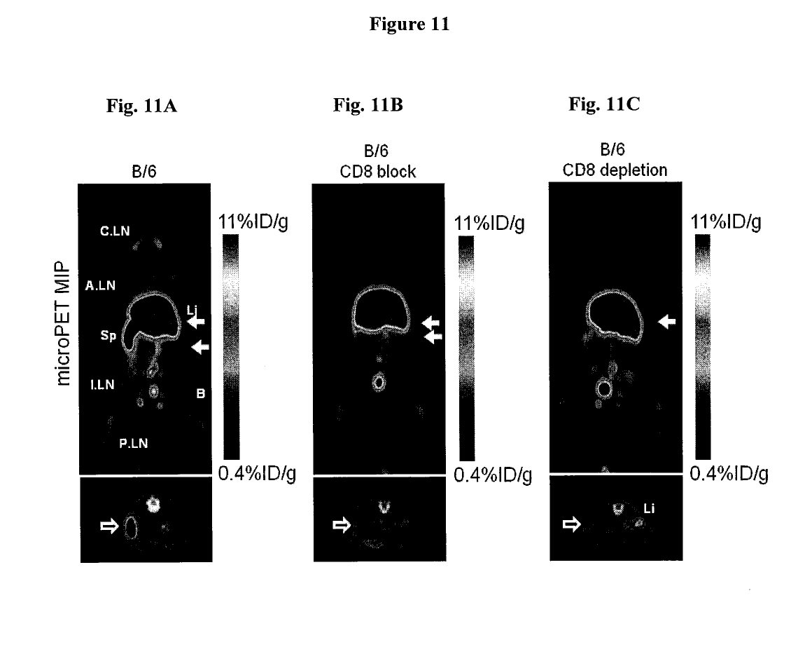

The ⁶⁴Cu-NOTA-2.43 minibody was then injected into B/6 mice that were blocked with a co-injection of 80 μg (4 mg/kg) cold 2.43 minibody or had received anti-CD8 antibody depletion therapy (16 mg/kg for three consecutive days). ImmunoPET images and ex vivo biodistribution acquired 4 hrs post-injection of antigen-blocked and antigen-depleted mice (FIG. 11 and Table 3) showed similar uptake as the antigen-negative C3H and NSG mice. CD8 depletion was confirmed using flow cytometry as described above.

### Example 4

### Production of Four Rat Anti-Mouse CD8 Antibodies

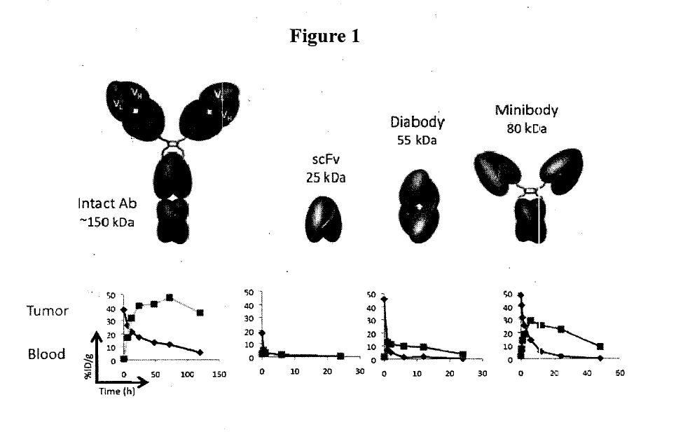

The variable regions, V_{H} and V_{L}, of four rat anti-mouse CD8 (mCD8; YTS 105.18.10, YTS 169.4.2.1, YTS 156.7.7, and 2.43) antibodies were sequenced for reformatting to various antibody fragments, including for example single chain variable fragments, diabodies and minibodies. For radionuclide or fluorescent base whole body imaging of CD8 expression, the engineering into antibody fragments serves two purposes: firstly, antibody fragments do not contain the complete Fc domain and will, therefore, not deplete CD8 expressing cells in vivo, and secondly, the antibody fragments have optimal pharmacokinetics for imaging purpose because the lack of a full Fc domain decreases the blood half-life. A reduction of blood half-life allows for high signal-to-noise images at earlier time points (FIGS. 1-3). In the initial disclosed herein, anti-mCD8 minibody antibody fragments were engineered, produced, purified and conjugated to the chelator NOTA for radiolabeling and immunoPET imaging with the radioisotope ⁶⁴Cu. However, this technology is not restricted to engineered minibodies listed here, as all other antibody fragments can be engineered.

In this study, the V_{H} and V_{L} domains of the parental antibodies from the hybridomas YTS 105.18.10 (YTS 105), YTS 156.7.7 (YTS 156), YTS 169.4.2.1 (YTS 169) and 2.43 were amplified using reverse transcription PCR (RT-PCR) for subsequent sequencing. The YTS 105, YTS 169 and 2.43 antibodies all bind mCD8a. They differ, however, because the YTS 105 and YTS 169 antibodies bind both Lyt2.1 and Lyt2.2 while the 2.43 antibody binds an epitope that is Lyt2.2 specific (Table 3). The YTS 156 parental antibody binds CD8b. For V_{H} and V_{L} sequence validation, the recovered RT-PCR sequences from hybridomas YTS 105 and YTS 156 were compared to the published crystal structures. For the hybridoma 2.43, the obtained V_{H} and V_{L} sequences were confirmed with trypsin digest-mass spectrometry analysis of purified intact antibody. The YTS 169 hybridoma was engineered into antibody fragments without further V_{H} and V_{L} validation.

TABLE 3
Murine CD8 binding by different anti-mCD8 hybridomas.

| Hybridoma | CD8a Lyt2.1 | CD8α Lyt2.2 | CD8β Lyt3 |
|---|---|---|---|
| YTS 105.18.10 | Yes | Yes | No |
| YTS 169.4.2.1 | Yes | Yes | No |
| 2.43 | No | Yes | No |
| YTS 156.7.7 | No | No | Yes |

Using the V_{H} and V_{L} sequences obtained from RT-PCR for three of the four sequenced antibodies (2.43, YTS 156 and YTS 169) to target various CD8 epitopes, minibody fragments were engineered, subcloned into the pEE12 expression vector and expressed in NS0 cells (FIG. 2). The inclusion of a C-terminal HisTag allows for the facile NiNTA purification of cell supernatant using imidazole elution.

After purification, functional binding was verified by flow cytometry on BW58 (Lyt2.2⁺, Lyt3⁺), TK-1 (Lyt2.1⁺, Lyt3⁺) or EL-4 (CD8) murine lymphoma cell lines. Purified Mbs were shown to bind the soluble CD8ab (sCD8ab) Lyt2.2⁺ fusion protein using size exclusion chromatography.

The YTS 169 and 2.43 minibody were then conjugated to S-2-(4-Isothiocyanatobenzyl)-1,4,7-triazacyclononane-1,4,7-triacetic acid (p-SCN-Bn-NOTA) for ⁶⁴Cu radiolabeling for immunoPET imaging and biodistribution studies. Both Mb constructs show uptake in the spleen, bone marrow and lymph nodes, as well as non-specific uptake in PET images and biodistribution studies 4 hours post-injection. For the 2.43 minibody that binds the Lyt2.2 epitope, the percent-injected dose per gram (% ID/g) in the spleen decreased 4.5-fold from 69.2 to 15.4 in wild type BL/6 mice (Lyt2.2⁺) than wild type C3H mice (Lyt2.1⁺). Furthermore, axillary lymph node and bone uptake was decreased 12.2- and 1.5-fold, respectively. The NOTA conjugated YTS 169 minibody showed similar uptake in the spleen and bone of wild type C57BL/6, 48.9 and 7.9% ID/g respectively.

These studies demonstrate the initial steps for developing a functional CD8 imaging agent based on engineered antibodies for use in a variety of preclinical disease and immunotherapeutic models.

### Example 5

### ImmunoPET Imaging of CD8 Expression Using an Anti-CD8 Diabody

Antibodies and their engineered fragments, such as diabodies, are well suited for ImmunoPET imaging of cell surface proteins in vivo. Due to their rapid clearance, diabodies have been shown to enable high target to background PET images for antigen expression at much shorter times (4-44 hrs) than intact antibodies (5-7 days). Furthermore, the diabody antibody fragment does not contain an Fc domain and therefore lacks Fc dependent effector functions that are not desired in an imaging agent. Disclosed herein, the variable domains of a depleting parental rat anti-mouse CD8 antibody (clone 2.43) have been reformatted into a Cys-diabody (cDb; ˜51 kDa) for the targeting of murine CD8 in a range of animal models. The 2.43 cDb binds specifically CD8-Lyt2.2 that is expressed on the mouse strains C57BL/6 and BALB/c but not CD8-Lyt2.1 expressed on C3H and CBA/Ca mice. ImmunoPET imaging of CD8 expression in preclinical models can thus provide the ability to monitor therapies not only to CD8⁺ T cell lymphoma models, but also to monitor the tumor infiltration of CD8⁺ T cells in the context of immunotherapies, such as T cell and/or dendritic cell adoptive transfer and other antibody-based immunotherapeutics.

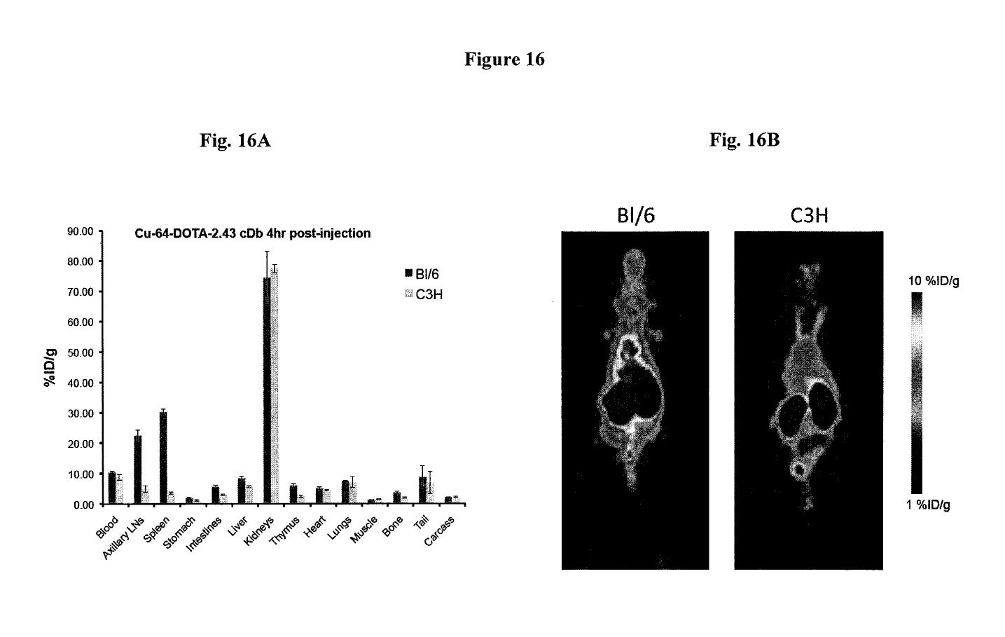

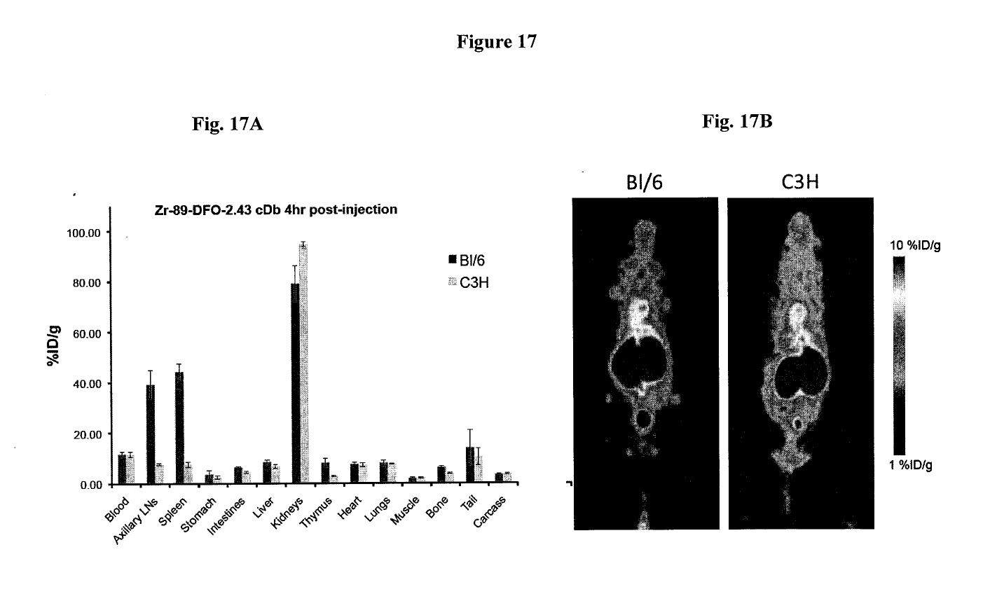

In this study, the 2.43 cDb was site-specifically conjugated to either maleimide-DOTA or maleimide-DFO for the subsequent radiolabeling and ImmunoPET imaging with ⁶⁴Cu or ⁸⁹Zr, respectively. Both the ⁶⁴Cu-DOTA-2.43 cDb (3.2 μCi/μg, ˜10 μg injection) and the ⁸⁹Zr-DFO-2.43 cDb (3.4 μCi/μg, ˜10 μg injection) constructs targeted the spleen and lymph nodes of antigen-positive C57Bl/6 mice (Lyt2.2⁺) but not antigen-negative C3H mice (Lyt2.1⁺) at 4 hours post-injection (FIGS. 16 and 17). Axillary lymph node and spleen uptake for ⁶⁴Cu and ⁸⁹Zr radiolabeled 2.43 cDb ranged from 22.5(⁶⁴Cu)-39.3(⁸⁹Zr) % ID/g and 30.2(⁶⁴Cu)-44.2(⁸⁹Zr) % ID/g, respectively (FIGS. 16 and 17). Axillary lymph node- and spleen-to-blood ratios for ⁶⁴Cu and ⁸⁹Zr radiolabeled 2.43 cDb varied from 2.2(⁶⁴Cu)-3.4(⁸⁹Zr) and 2.9(⁶⁴Cu)-3.8(⁸⁹Zr), respectively, in antigen-positive C57Bl/6 mice and 0.5(⁶⁴Cu)-0.7(⁸⁹Zr) to 0.4(⁶⁴Cu)-0.7(⁸⁹Zr) in antigen-negative C3H mice, demonstrating specific uptake in the lymphoid organs of Lyt2.2⁺ mice (FIGS. 16 and 17). At only 4 hours post-injection, there is still ˜10% ID/g in the blood so images acquired at later time points will allow for more blood clearance and should yield higher target-to-background ImmunoPET images. These initial results show the abilities and benefits of non-invasive monitoring of immunotherapeutics in vivo.

1. An antibody that binds to CD8 comprising:
The V_{H} domain of any of SEQ ID NOs.: 7, 15, or 23, and
The V_{L} domain of any of SEQ ID NOs.: 11, 19, or 27.

2. The antibody of claim 1 comprising the V₁₁ domain of SEQ ID NO.: 7 and the V_{L} domain of any of SEQ ID NO.: 11.

3. The antibody of claim 1 comprising the V_{H} domain of SEQ ID NO.: 15 and the V_{L} domain of any of SEQ ID NO.: 19.

4. The antibody of claim 1 comprising the V_{H} domain of SEQ ID NO.: 23 and the V_{L} domain of any of SEQ ID NO.: 27.

5. An antibody that binds to CD8 comprising:
A heavy chain CDR1 of any of SEQ ID NOs.: 8, 16, or 24,
A heavy chain CDR2 of any of SEQ ID NOs.: 9, 17, or 25,
A heavy chain CDR3 of any of SEQ ID NOs.: 10, 18, or 26,
A light chain CDR1 of any of SEQ ID NOs.: 12, 20, or 28,
A light chain CDR2 of any of SEQ ID NOs.: 13, 21, or 29, and
A light chain CDR3 of any of SEQ ID NOs.: 14, 22, or 30.

6. An antibody of claim 5 comprising:
A heavy chain CDR1 of SEQ ID NO.: 8,
A heavy chain CDR2 of SEQ ID NO.: 9,
A heavy chain CDR3 of SEQ ID NO.: 10,
A light chain CDR1 of SEQ ID NO.: 12,
A light chain CDR2 of SEQ ID NO.: 13, and
A light chain CDR3 of SEQ ID NO.: 14.

7. An antibody of claim 5 comprising:
A heavy chain CDR1 of SEQ ID NO.: 16,
A heavy chain CDR2 of SEQ ID NO.: 17,
A heavy chain CDR3 of SEQ ID NO.: 18,
A light chain CDR1 of SEQ ID NO.: 20,
A light chain CDR2 of SEQ ID NO.: 21, and
A light chain CDR3 of SEQ ID NO.: 22.

8. (canceled)

9. The antibody of claim 5 selected from the group consisting of scFv, scFv dimer (diabody), scFv-C_{H}3 dimer (minibody) and scFv-Fc.

10. The antibody of claim 9 wherein the antibody is a diabody.

11. The antibody of claim 9 wherein the antibody is a minibody.

12. The antibody of claim 5 wherein the scFv dimer comprises two scFv monomers joined by a linker.

13. The antibody of claim 12 wherein the linker is a peptide sequence.

14. The antibody of claim 13 wherein the linker is selected from the group consisting of GGGS, GGGSGGGS, and GSTSGGGSGGGSGGGGSS.

15. The antibody of claim 5 wherein the antibody is a humanized antibody fragment which binds to CD8.

16. The antibody of claim 5 wherein the antibody is a chimeric antibody.

17. The antibody of claim 5 wherein the antibody is linked to a detectable moiety.

18. The antibody of claim 17 wherein the detectable moiety is selected from the group consisting of a radionuclide, a nanoparticle, a fluorescent dye, a fluorescent marker, and an enzyme.

19. The antibody of claim 17 wherein the detectable moiety is a radionuclide.

20. The antibody of claim 19 wherein the radionuclide is selected from the group consisting ⁴⁷Sc, ⁸⁹Sr, ¹⁰⁵Rh, ¹¹¹Ag, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, ¹⁸F, ¹²⁴I, ¹²⁵I, ¹³¹I, ⁵⁵Co, ⁶⁰Cu, ⁶¹Cu, ⁶²Cu, ⁶⁴Cu, ⁶⁶Ga, ⁶⁷Cu, ⁶⁷Ga, ⁶⁸Ga, ⁸²Rb, ⁸⁶Y, ⁸⁷Y, ⁹⁰Y, ¹¹¹In, ⁹⁹Tc, and ²⁰¹Tl.

21. The antibody of claim 17 wherein the detectable moiety can be imaged in vivo using a system selected from the group consisting of MRI, SPECT, PET, and planar gamma camera imaging.

22. A method of diagnosing a CD8 mediated disease, the method comprising the steps of:
a. administering to a subject an antibody comprising a light chain CDR1 of any of SEQ ID NOs.: 12, 20, or 28; a light chain CDR2 of any of SEQ ID NOs.: 13, 21, or 29; a light chain CDR3 of any of SEQ ID NOs.: 14, 22, or 30; a heavy chain CDR1 of any of SEQ ID NOs.: 8, 16, or 24; a heavy chain CDR2 of any of SEQ ID NOs.: 9, 17, or 25; and a heavy chain CDR3 of any of SEQ ID NOs.: 10, 18, or 26, which antibody specifically binds to the CD8 on the surface of cells, to a subject;
b. determining the expression of the CD8 protein in the subject using molecular in vivo imaging; and
c. determining whether or not CD8 protein is overexpressed in the subject using molecular in vivo imaging;
wherein an overexpression of CD8 indicates a CD8 mediated disease.

23. The method of claim 22 wherein the heavy chain CDR1 has the sequence comprising the amino acid sequence of SEQ ID NO.: 8, the heavy chain CDR2 has the sequence comprising the amino acid sequence of SEQ ID NO.: 9, the heavy chain CDR3 has the sequence comprising the amino acid sequence of SEQ ID NO.: 10, the light chain CDR1 has the sequence comprising the amino acid sequence of SEQ ID NO.: 12, the light chain CDR2 has the sequence comprising the amino acid sequence of SEQ ID NO.: 13, and the light chain CDR3 has the sequence comprising the amino acid sequence of SEQ ID NO.: 14.

24. The method of claim 22 wherein the heavy chain CDR1 has the sequence comprising the amino acid sequence of SEQ ID NO.: 16, the heavy chain CDR2 has the sequence comprising the amino acid sequence of SEQ ID NO.: 17, the heavy chain CDR3 has the sequence comprising the amino acid sequence of SEQ ID NO.: 18, the light chain CDR1 has the sequence comprising the amino acid sequence of SEQ ID NO.: 20, the light chain CDR2 has the sequence comprising the amino acid sequence of SEQ ID NO.: 21, and the light chain CDR3 has the sequence comprising the amino acid sequence of SEQ ID NO.: 22.

25. (canceled)

26. The method of claim 22 wherein the CD8 mediated disease is cancer.

27. The method of claim 22 wherein the CD8 mediated disease is an autoimmune disease.

28.-40. (canceled)

41. A method of providing the prognosis for a CD8 mediated disease, the method comprising the steps of:
a. administering to a subject an antibody comprising a light chain CDR1 of any of SEQ ID NOs.: 12, 20, or 28; a light chain CDR2 of any of SEQ ID NOs.: 13, 21, or 29; a light chain CDR3 of any of SEQ ID NOs.: 14, 22, or 30; a heavy chain CDR1 of any of SEQ ID NOs.: 8, 16, or 24; a heavy chain CDR2 of any of SEQ ID NOs.: 9, 17, or 25; and a heavy chain CDR3 of any of SEQ ID NOs.: 10, 18, or 26, which antibody specifically binds to the CD8 on the surface of cells, to a subject;
b. determining the expression of the CD8 protein in the subject using molecular in vivo imaging; and
c. determining whether or not the CD8 protein is overexpressed in the subject using molecular in vivo imaging;
wherein an overexpression of CD8 indicates a CD8 mediated disease, wherein the degree of overexpression of CD8 indicates the prognosis for the CD8 mediated disease, and wherein the greater the expression the poorer the prognosis.

42. The method of claim 41 wherein the heavy chain CDR1 has the sequence comprising the amino acid sequence of SEQ ID NO.: 8, the heavy chain CDR2 has the sequence comprising the amino acid sequence of SEQ ID NO.: 9, the heavy chain CDR3 has the sequence comprising the amino acid sequence of SEQ ID NO.: 10, the light chain CDR1 has the sequence comprising the amino acid sequence of SEQ ID NO.: 12, the light chain CDR2 has the sequence comprising the amino acid sequence of SEQ ID NO.: 13, and the light chain CDR3 has the sequence comprising the amino acid sequence of SEQ ID NO.: 14.

43. The method of claim 41 wherein the heavy chain CDR1 has the sequence comprising the amino acid sequence of SEQ ID NO.: 16, the heavy chain CDR2 has the sequence comprising the amino acid sequence of SEQ ID NO.: 17, the heavy chain CDR3 has the sequence comprising the amino acid sequence of SEQ ID NO.: 18, the light chain CDR1 has the sequence comprising the amino acid sequence of SEQ ID NO.: 20, the light chain CDR2 has the sequence comprising the amino acid sequence of SEQ ID NO.: 21, and the light chain CDR3 has the sequence comprising the amino acid sequence of SEQ ID NO.: 22.

44.-60. (canceled)

61. The antibody of claim 5 wherein the antibody comprises the amino acid sequence of SEQ ID NO.: 2.

62. The antibody of claim 5 wherein the antibody comprises the amino acid sequence of SEQ ID NO.: 4.

63. (canceled)

64. The antibody of claim 5 wherein the antibody comprises the amino acid sequence of SEQ ID NO.: 39.

65. The antibody of claim 5 wherein the antibody comprises the amino acid sequence of SEQ ID NO.: 43.

66. (canceled)

67. The antibody of claim 5 wherein the antibody comprises the amino acid sequence of SEQ ID NO.: 41.

68. The antibody of claim 5 wherein the antibody comprises the amino acid sequence of SEQ ID NO.: 45.

69.-81. (canceled)
